SEQUENCE LISTING (1 sequence)

SEQ ID NO: 1 - protein, 254 aa, Artificial Sequence
  modified CD16
MWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVPTISSFFPPGYQVSFCLVMVLLFAVDTGLYFSVKTNIRSSTRDWKDHKFKWRKDPQDK

(19) United States
(12) Patent Application Publication
(10) Pub. No.: US 2018/0273903 A1
(43) Pub. Date: Sep. 27, 2018

(54) GENETICALLY MODIFIED NATURAL KILLER CELLS

(71) Applicant: Celularity, Inc., Warren, NJ (US)

(72) Inventors: Xiaokui Zhang, Martinsville, NJ (US); Qian Ye, Martinsville, NJ (US); Tianjian Li, Belle Mead, NJ (US); Chuan Wang, Montville, NJ (US); Mini Bharathan, Berkeley Height, NJ (US); Uri Herzberg, Bridgewater, NJ (US)

(73) Assignee: Celularity, Inc., Warren, NJ (US)

(21) Appl. No.: 15/857,516

(22) Filed: Dec. 28, 2017

Related U.S. Application Data
(60) Provisional application No. 62/440,909, filed on Dec. 30, 2016.

(51) Int. Cl. C12N 5/0783; A61K 35/17; A61P 35/00; A61K 45/06; C07K 14/735

(52) U.S. Cl. CPC C12N 5/0646; A61K 35/17; C07K 14/70535; A61K 45/06; A61P 35/00

(57) ABSTRACT

Provided herein are genetically modified (GM) natural killer (NK) cells and methods of producing populations of GM NK cells. Further provided herein are methods of using the GM NK cells described herein, to, e.g., suppress the proliferation of tumor cells, or to inhibit pathogen infection, e.g., viral infection. In certain alternatives, GM NK cells provided herein lack expression of CBLB, NKG2A and/or TGFBR2 and/or function or show reduced expression and/or function of CBLB, NKG2A and/or TGFBR2. In certain alternatives, GM NK cells provided herein comprise modified CD16.

### INCORPORATION BY REFERENCE TO ANY PRIORITY APPLICATIONS

The present application claims the benefit of priority to U.S. Provisional Patent Application No. 62/440,909, filed Dec. 30, 2016. The entire disclosure of the aforementioned application is hereby expressly incorporated by reference in its entirety.

### I. FIELD

Described herein are genetically modified (GM) natural killer (NK) cells and methods of producing cell populations that include GM NK cells. Also disclosed are methods of using these cell populations that include the GM NK cells to, e.g., suppress the proliferation of tumor cells, modulate pathogen infection, such as bacterial infection, or viral infection, or to inhibit pathogen infection, e.g., bacterial infection, or viral infection. In certain alternatives, the population of cells that include GM NK cells lack expression of CBLB, NKG2A and/or TGFBR2 and/or exhibit a reduced expression and/or function of CBLB, NKG2A and/or TGFBR2. In certain alternatives, the cell population includes GM NK cells, which comprise modified CD16.

### II. BACKGROUND

Natural killer (NK) cells are cytotoxic lymphocytes that constitute a major component of the innate immune system.

NK cells are activated in response to interferons or macrophage-derived cytokines. The cytotoxic activity of NK cells is largely regulated by two types of surface receptors, which may be considered “activating receptors” or “inhibitory receptors,” although some receptors, e.g., CD94 and 2B4 (CD244), work either way depending on ligand interactions.

Among other activities, NK cells play a role in the host rejection of tumors and have been shown to be capable of killing virus-infected cells. Natural killer cells may become activated by cells lacking, or displaying reduced levels of, major histocompatibility complex (MHC) proteins. Cancer cells with altered or reduced level of self-class I MHC expression may result in induction of NK cell sensitivity. Activated and expanded NK cells, and in some cases LAK cells, from peripheral blood have been used in both ex vivo therapy and in vivo treatment of patients having advanced cancer, with some success against bone marrow related diseases, such as leukemia; breast cancer; and certain types of lymphoma. More approaches to develop modified NK cells are needed.

### III. SUMMARY

Described herein are genetically modified (GM) natural killer (NK) cells, for example, human NK cells, methods of producing populations of cells that comprise GM NK cells, and methods of using the GM NK cells or populations of cells that comprise the GM NK cells described herein, to, e.g., suppress the proliferation of tumor cells, modulate pathogen infection (e.g., bacterial infection, or viral infection) or to inhibit pathogen infection, e.g., bacterial infection, or viral infection.

In some alternatives, a population of NK cells is provided, wherein the NK cells are genetically modified such that they lack expression of an NK inhibitory molecule or manifest a reduced expression of an NK inhibitory molecule. In some alternatives, the NK cells are genetically modified such that they modulate expression of an NK inhibitory molecule or inhibit the expression of an NK inhibitory molecule. For example, in some alternatives, the modified NK cells provided herein include a population of cells comprising NK cells, which have been genetically modified to express one or more NK inhibitory molecules at a lower level than NK cells that are not modified with respect to expression levels of the NK inhibitory molecules (such cells are referred to herein as “unmodified cells” even though such cells may be modified from naturally occurring cells in respects other than expression of NK inhibitory molecules). The unmodified cells to which the levels of NK inhibitory molecules are compared can be, for example, naturally occurring NK cells or NK cells that are obtained using methods such as those described herein and are not naturally occurring. In certain alternatives, the NK inhibitory molecule which is expressed at a modulated, reduced, or null level is CBLB, NKG2A and/or TGFBR2.

In certain alternatives, the NK inhibitory molecule, which is expressed at a modulated, reduced, or null level in the NK cells, is CBLB. In certain alternatives, the CBLB expression in the NK cells has been knocked out. In certain alternatives, the CBLB expression in the NK cells has been knocked out by a gene editing technique, such as by using CRISPR or a CRISPR-related technique. In certain alternatives, the knockout of CBLB expression in the NK cells generates a population of NK cells or a population of cells comprising NK cells having a higher cytotoxicity against tumor cells than NK cells in which CBLB has not been knocked out, which may be naturally occurring NK cells or non-naturally occurring NK cells that have not been genetically modified to reduce or eliminate expression of CBLB. In specific alternatives, the tumor cells are multiple myeloma cells. In specific alternatives, the tumor cells are RPMI8226 cells. In specific alternatives, the tumor cells are U266 cells. In specific alternatives, the tumor cells are ARH77 cells. In specific alternatives, the tumor cells are acute myeloid leukemia (AML) cells. In specific alternatives, the tumor cells are HL60 cells. In specific alternatives, the tumor cells are KG1 cells. In certain alternatives, the knockout of CBLB expression in the NK cells generates a population of NK cells having a higher IFNγ secretion than unmodified NK cells, wherein CBLB has not been knocked out e.g., naturally occurring NK cells. In certain alternatives, the knockout of CBLB expression in the NK cells generates a population of NK cells having a higher degranulation than NK cells in which CBLB has not been knocked out. In specific alternatives, the degranulation is measured by an increase in CD107a. In certain alternatives, the knockout of CBLB expression in the NK cells generates a population of NK cells having a change in the secretion of one or more of GM-CSF, soluble CD137 (sCD137), IFNγ, MIP1α, MIP1β, TNFα or perform, as compared to NK cells in which CBLB has not been knocked out. In certain alternatives, the knockout of CBLB expression in the NK cells generates a population of NK cells having a change in the secretion of one or more of GM-CSF, soluble CD137 (sCD137), IFNγ, MIP1α, MIP1β, TNFα or perform, as compared to NK cells in which CBLB has not been knocked out, such as naturally occurring NK cells.

In certain alternatives, the NK inhibitory molecule that is modulated or is reduced in expression in the population of cells comprising NK cells is NKG2A. In certain alternatives, the NKG2A expression has been knocked out. In certain alternatives, the NKG2A expression has been knocked out by CRISPR or a CRISPR-related technique. In certain alternatives, the knockout of NKG2A expression in the NK cells generates a population of cells comprising NK cells having a higher cytotoxicity against tumor cells than NK cells in which NKG2A has not been knocked out, such as naturally occurring NK cells. In specific alternatives, the tumor cells are multiple myeloma cells. In specific alternatives, the tumor cells are RPMI8226 cells. In specific alternatives, the tumor cells are U266 cells. In specific alternatives, the tumor cells are ARH77 cells. In certain alternatives, the knockout of NKG2A expression in the NK cells generates a population of NK cells with higher IFNγ secretion than NK cells in which NKG2A has not been knocked out. In certain alternatives, secreted IFNγ is measured from NK cells stimulated with ICAM-1 and MICA in the presence of an agonist NKG2A antibody in vitro. In certain alternatives, the knockout of NKG2A expression in the NK cells generates a population of NK cells with higher degranulation than NK cells in which NKG2A has not been knocked out. In specific alternatives, the degranulation is measured by an increase in CD107a. In certain alternatives, the knockout of NKG2A expression in the NK cells generates a population of NK cells with a change in the secretion of one or more of GM-CSF, soluble CD137 (sCD137), IFNγ, MIP1α, MIP1β, TNFα or perform, as compared to NK cells in which NKG2A has not been knocked out. In some alternatives herein, NKG2A knockout NK cells have up to a three-fold or more increase in cytotoxicity in comparison to untreated cells that have no NKG2A knockout, such as naturally occurring NK cells.

In certain alternatives, the NK inhibitory molecule which is modulated or reduced in expression in the population of cells comprising NK cells is TGFBR2. In certain alternatives, the TGFBR2 expression in the population of cells comprising NK cells has been knocked out. In certain alternatives, the TGFBR2 expression has been knocked out by CRISPR or a CRISPR-related technique. In certain alternatives, the knockout of TGFBR2 expression in the NK cells generates a population of cells that are resistant to TGFβ mediated inhibition of NK cells cytotoxicity against tumor cells, as compared to NK cells in which TGFBR2 has not been knocked out. In specific alternatives, the tumor cells are multiple myeloma cells. In specific alternatives, the tumor cells are RPMI8226 cells. In specific alternatives, the tumor cells are K562 cells. In specific alternatives, the tumor cells are HL-60 cells.

In certain alternatives, a population of natural killer cells is provided, wherein the natural killer (NK) cells are genetically modified to comprise a modified CD16, for example, a modified CD16a. In certain alternatives, the modified CD16 has a higher affinity for IgG than wildtype CD16, for example, the modified CD16a has a higher affinity for IgG than wildtype CD16a. In certain alternatives, the modified CD16 has a valine at position 158 of CD16a. In certain alternatives, the modified CD16 is resistant to ADAM17 cleavage. In certain alternatives, the CD16 has a proline at position 197 of CD16a. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In certain alternatives, the modified CD16 contains an IgK signal peptide. In certain alternatives, the modified CD16 comprises a CD16 signal peptide. In certain alternatives, the modified CD16 is introduced into the NK cells via viral infection. In certain alternatives, the modified CD16 is introduced into hematopoietic cells via viral infection, which hematopoietic cells are then differentiated into NK cells. In certain alternatives, the modified CD16 is introduced via a lentiviral vector. In certain alternatives, the lentiviral vector has either a CMV or an EF1α promoter. In certain alternatives, the lentiviral vector comprises one or more drug selection markers. In certain alternatives, the modified CD16 is introduced via a retroviral vector. In certain alternatives, the retroviral vector comprises one or more drug selection markers.

Described herein are methods of suppressing the proliferation of tumor cells comprising contacting the tumor cells with one or more populations of genetically modified natural killer cells prepared as described herein. In certain alternatives, said contacting takes place in vitro. In certain alternatives, said contacting takes place in vivo. In certain alternatives, said contacting takes place in a human individual. In certain alternatives, the human individual is selected or identified as one in need for a cancer therapy. In certain alternatives, said method comprises administering said natural killer cells to said selected or identified individual. In certain alternatives, said tumor cells are multiple myeloma cells. In certain alternatives, said tumor cells are acute myeloid leukemia (AML) cells. In certain alternatives, said individual has relapsed/refractory AML. In certain alternatives, said individual has AML that has failed at least one non-innate lymphoid cell (ILC) therapeutic against AML. In certain alternatives, said individual is 65 years old or greater, and is in first remission. In certain alternatives, said individual has been conditioned with fludarabine, cytarabine, or both, prior to administering said natural killer cells. In certain alternatives, said tumor cells are breast cancer cells, head and neck cancer cells, or sarcoma cells. In certain alternatives, said tumor cells are primary ductal carcinoma cells, leukemia cells, acute T cell leukemia cells, chronic myeloid lymphoma (CML) cells, chronic myelogenous leukemia (CML) cells, multiple myeloma (MM) cells, lung carcinoma cells, colon adenocarcinoma cells, histiocytic lymphoma cells, colorectal carcinoma cells, colorectal adenocarcinoma cells, and/or retinoblastoma cells. In certain alternatives, said tumor cells are solid tumor cells. In certain alternatives, said tumor cells are liver tumor cells. In certain alternatives, said tumor cells are lung tumor cells. In certain alternatives, said tumor cells are pancreatic tumor cells. In certain alternatives, said tumor cells are renal tumor cells. In certain alternatives, said tumor cells are glioblastoma multiforme (GBM) cells.

In certain alternatives, said natural killer cells are administered in conjunction with an anti-CD33 antibody. In certain alternatives, said natural killer cells are administered in conjunction with an anti-CD20 antibody. In certain alternatives, said natural killer cells are administered in conjunction with an anti-CD138 antibody. In certain alternatives, said natural killer cells are administered in conjunction with an anti-CD38 antibody. In certain alternatives, said natural killer cells are administered in conjunction with an anti-CD32 antibody.

In certain alternatives, said natural killer cells have been cryopreserved prior to said contacting or said administering. In certain alternatives, said natural killer cells have not been cryopreserved prior to said contacting or said administering.

In certain alternatives, said natural killer cells are CD56⁺CD3⁻CD117⁺CD11a⁺, express perform and/or EOMES, and do not express one or more of RORγt, aryl hydrocarbon receptor, and/or IL1R1. In certain alternatives, said natural killer cells express perform and/or EOMES, and do not express any of RORγt, aryl hydrocarbon receptor, and/or IL1R1. In certain alternatives, said natural killer cells additionally express T-bet, GZMB, NKp46, NKp30, and/or NKG2D. In certain alternatives, said natural killer cells express CD94. In certain alternatives, said natural killer cells do not express CD94.

In a first aspect, a population of natural killer cells is provided, wherein the natural killer (NK) cells are genetically modified to lack expression of an NK inhibitory molecule or manifest a reduced expression of an NK inhibitory molecule. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out. In some alternatives, the NK inhibitory molecule is TGFBR2. In some alternatives, the knockout of TGFBR2 expression generates a population of NK cells having a resistance to TGFβ mediated inhibition of NK cell cytotoxicity against tumor cells compared to NK cells in which TGFBR2 has not been knocked out. In some alternatives, the natural killer (NK) cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the modified CD16 has a proline at position 197 of CD16a. In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the NK cells are placenta derived (PNK cells). In some alternatives, the natural killer cells are CD56+CD3−CD117+CD11a+, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives, said natural killer cells additionally express T-bet, GZMB, NKp46, NKp30, and/or NKG2D. In some alternatives, said natural killer cells express CD94. In some alternatives, said natural killer cells do not express CD94.

In a second aspect, a method of suppressing the proliferation of tumor cells comprising contacting the tumor cells with natural killer cells from the population of any one of the alternative population of natural killer cells herein are provided. In some alternatives, the population of natural killer cells is provided, wherein the natural killer (NK) cells are genetically modified to lack expression of an NK inhibitory molecule or manifest a reduced expression of an NK inhibitory molecule. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out. In some alternatives, the NK inhibitory molecule is TGFBR2. In some alternatives, the knockout of TGFBR2 expression generates a population of NK cells having a resistance to TGFβ mediated inhibition of NK cell cytotoxicity against tumor cells compared to NK cells in which TGFBR2 has not been knocked out. In some alternatives, the natural killer (NK) cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the modified CD16 has a proline at position 197 of CD16a. In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the NK cells are placenta derived (PNK cells). In some alternatives, the natural killer cells are CD56+CD3−CD117+CD11a+, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives, said natural killer cells additionally express T-bet, GZMB, NKp46, NKp30, and/or NKG2D. In some alternatives, said natural killer cells express CD94. In some alternatives, said natural killer cells do not express CD94. In some alternatives of the method, said contacting takes place in vitro. In some alternatives of the method, said contacting takes place in vivo. In some alternatives of the method, said contacting takes place in a human individual, preferably an individual selected to receive an anticancer therapy. In some alternatives of the method, said method comprises administering said natural killer cells to said individual. In some alternatives of the method, said tumor cells are multiple myeloma cells. In some alternatives of the method, said tumor cells are acute myeloid leukemia (AML) cells. In some alternatives of the method, said individual has relapsed/refractory AML. In some alternatives of the method, said individual has AML that has failed at least one non-innate lymphoid cell (ILC) therapeutic against AML. In some alternatives of the method, said individual is 65 years old or greater, and is in first remission. In some alternatives of the method, said individual has been conditioned with fludarabine, cytarabine, or both, prior to administering said natural killer cells. In some alternatives of the method, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives of the method, the tumor cells are solid tumor cells. In some alternatives of the method, the solid tumor cells are selected from the group consisting of liver tumor cells, lung tumor cells, pancreatic tumor cells, renal tumor cells, and glioblastoma multiforme (GBM) cells. In some alternatives of the method the natural killer cells are administered with an anti-CD33 antibody. In some alternatives of the method, said natural killer cells are administered with an anti-CD20 antibody. In some alternatives of the method, said natural killer cells are administered with an anti-CD138 antibody. In some alternatives of the method, said natural killer cells are administered with an anti-CD38 antibody. In some alternatives of the method, said natural killer cells have been cryopreserved prior to said contacting or said administering. In some alternatives of the method, said natural killer cells have not been cryopreserved prior to said contacting or said administering.

In a third aspect, a population of natural killer cells derived from placenta or parts thereof, thereby comprising placenta derived NK cells (pNK cells), wherein the pNK cells are genetically modified such that they lack expression of an NK inhibitory molecule or manifest reduced expression of an NK inhibitory molecule, are provided. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives expression of the NK inhibitory molecule has been knocked out. In some alternatives, expression of the NK inhibitory molecule has been knocked out by CRISPR/CAS9 system, a zinc finger nuclease or TALEN nuclease. In some alternatives expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and/or perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the increase in CD107a is measured by FACs. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out, such as naturally occurring NK cells.

In a fourth aspect, a population of placental derived natural killer cells (pNK), wherein the pNK cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the CD16 has a proline at position 197 of CD16a. In some alternatives, the modified CD16 contains an IgK signal peptide or CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the selection marker include genes encoding a protein conferring resistance to a selection agent such as PuroR gene, ZeoR gene, HygroR gene, neoR gene, and/or the blasticidin resistance gene. In some alternatives, the modified CD16 is introduced via a retroviral vector. In some alternatives, the retroviral vector comprises one or more drug selection markers.

### IV. BRIEF DESCRIPTION OF THE DRAWINGS

FIG. 1A-B: CBLB knock out efficiency in GM NK cells (1A) and (1B) fold-expansion post-knockout.

FIG. 2A-C: Cytotoxicity (as measured by percent killing) of untreated (diamonds) and CBLB-knockout (squares) three-stage NK cells against (2A) RPMI8226, (2B) U266, and (2C) ARH77 cells at day 34/35 of the three-stage process, at effector:target (E:T) ratios of 20:1, 10:1, and 5:1.

FIG. 3A-C: Relative cytotoxicity of untreated (diamonds) and CBLB⁻ knockout (squares) three-stage NK cells against (3A) RPMI8226, 3(B) U266, and (3C) ARH77 cells at day 34/35 of the three-stage process, at effector:target (E:T) ratios of 20:1, 10:1, and 5:1.

FIG. 4A-B: Relative cytotoxicity of untreated (NT) and CBLB⁻ knockout (CBLB KO) three-stage NK cells against (4A) HL-60 and (4B) KG1 cells.

FIG. 5A-B: (5A) IFN-γ secretion assay and (5B) CD107a/degranulation assay of untreated (right) and CBLB⁻ knockout (left) three-stage NK cells upon Major-histocompatibility-complex (MHC) class I-related chain A (MICA) stimulation at varying amounts in the presence of 1.25 μg/ml of ICAM-1.

FIG. 6A-C: Levels of secreted cytokines during co-incubation with (6A) RPMI8226, (6B) U266, and (6C) ARH77 cells for CBLB knockout three-stage NK cells, expressed as a percentage of cytokine secretion by untreated three-stage NK cells.

FIG. 7: Schematic for CBLB knockout three-stage NK process.

FIG. 8: Number of human CD45⁺ cells in spleen, bone marrow (BM), blood, liver, lungs, and in total for NOD SCID gamma (NSG) mice day 7 post-administration of three-stage CBLB knock out NK cells, or untreated NK cells, with busulfan at day-1 or day-5.

FIG. 9: Number of human CD45⁺ cells in spleen, BM, blood, liver, lungs, and in total for NSG mice day 14 post-administration of CBLB knock out three-stage NK cells, or untreated NK cells, with busulfan at day-1 or day-5.

FIG. 10: Number of human CD45⁺ cells in spleen, BM, blood, liver, lungs, and in total for NSG mice day 21 post-administration of CBLB knock out three-stage NK cells, or untreated NK cells, with busulfan at day-1 or day-5.

FIG. 11A-D: Percent CD56^{±}CD11a⁺ three-stage NK cells in (11A) spleen, (11B) liver, (11C) bone marrow, and (11D) lungs of NSG mice at day 7, 14, and 21 post-administration with the CBLB knockout, or untreated, with busulfan at day-1 or day-5.

FIG. 12A-D: Percent CD56⁺CD16⁺ three-stage NK cells in (12A) spleen, (12B) liver, (12C) bone marrow, and (12D) lungs of NSG mice at day 7, 14, and 21 post-administration with the CBLB knockout, or untreated, with busulfan at day-1 or day-5.

FIG. 13A-D: Percent CD56⁺CD158b1,b2,j⁺ three-stage NK cells in (13A) spleen, (13B) liver, (13C) bone marrow, and (13D) lungs of NSG mice at day 7, 14, and 21 post-administration with the CBLB knockout, or untreated, with busulfan at day-1 or day-5.

FIG. 14A-B: Cytotoxicity of isolated, purified three-stage NK cells, CBLB knockout or control, 14 days post-administration from NSG mice against (14A) K562 and (14B) HL60 cells. Control shown as the lower percent killer in both (14A) and (14B).

FIG. 15A-D: (15A) GM-CSF, (15B) IFN-γ, (15C) sCD137, and (15D) TNF-α secretion of isolated, purified three-stage NK cells, CBLB knockout (right) or control (left), 14 days post-administration from NSG mice, co-incubated with K562 cells, HL60 cells, or no cells.

FIG. 16A-D: (16A) GM-CSF, (16B) IFN-γ, (16C) sCD137, and (16D) TNF-α secretion of three-stage NK cells, CBLB knockout (right) or control (left), 14 days post-administration from NSG mice co-cultured with two AML patient xenograft (PDX) tumor cells.

FIG. 17A-B: NKG2A knock out GM NK (17A) efficiency and (17B) fold-expansion post-knockout.

FIG. 18A-D: Cytotoxicity (as measured by percent killing) of untreated (diamonds) and NKG2A-knockout (squares) three-stage NK cells against (18A) K562, (18B) RPMI8226, (18C) U266, and (18D) ARH77 cells at day 34/35 of the three-stage process, at varying E:T ratios.

FIG. 19A-C: Relative cytotoxicity of untreated (diamonds) and NKG2A-knockout (squares) three-stage NK cells against (19A) RPMI8226, (19B) U266, and (19C) ARH77 cells at day 34/35 of the three-stage process, at effector:target (E:T) ratios of 20:1, 10:1, and 5:1.

FIG. 20: CD107a (plate bound) assay results for wild type three-stage NK cells with NKG2A antibody (squares), NKG2A knockout three-stage NK cells with NKG2A antibody (triangles), wildtype three-stage NK cells with IgG (circles), and NKG2A knockout three-stage NK cells with IgG (diamonds), all in the presence of 1.25 μg/ml ICAM-1 and 5 μg/ml MICA.

FIG. 21A-C: Levels of secreted cytokines during co-incubation with (21A) RPMI8226, (21B) U266, and (21C) ARH77 cells for NKG2A knockout three-stage NK cells, expressed as a percentage of cytokine secretion by untreated three-stage NK cells.

FIG. 22: Knockout efficiency for TGFBR2 knockout during 35-day three-stage NK process, upon transfection at day 5 (squares) versus day 10 (×s).

FIG. 23A-D: Cytotoxicity (as measured by percent killing) of three-stage NK cells versus tumor cell lines: (23A) control NK versus K562, (23B) TGFBR2 knockout versus K562, (23C) control NK versus RPMI8226, and (23D) TGFBR2 knockout versus RPMI8226, at varying E:T ratios, upon treatment with TGF-β1 at 20 ng/mL (squares) or 40 ng/mL (triangles) for 48 hours before assay, or left untreated (diamonds).

FIG. 24A-D: A four hour cytotoxicity assay in the absence (top line) or presence (bottom line) of TGF-β1, for (24A) control cells versus HL60 cells, (24B) TGFBR2 knockout cells versus HL60 cells, (24C) control cells versus K562 cells, and (24D) TGFBR2 knockout cells versus K562 cells.

FIG. 25: Persistence of CD16 expression in three-stage NK cells during culture for untreated or CD16VP transduced cells.

FIG. 26A-B: (26A) Fold expansion of three-stage NK cells left untreated (top line), or transduced with CD16VP (bottom line). (26B) Marker expression at day 33 of 35-day three-stage NK culture for untreated (left) or CD16VP transduced (right) cells.

FIG. 27A-B: ADCC mean specific killing for CD16VP transduced cells in the presence of (27A) anti-CD20 and (27B) anti-CD38 antibodies in a four hour ADCC assay against Daudi cells.

FIG. 28A-C show IFN-γ (FIG. 28A), GM-CSF (FIG. 28B), and TNF-α (FIG. 28C) secretion for CD16VP transduced cells in a four hour ADCC assay under various conditions.

FIG. 29: Fold expansion of double knock out three-stage GM NK, showing mock transfection (diamonds; 955.89), TGFBR2 single knock out (squares; 380), CBLB single knock out (triangles; 500.175), and TGFBR2/CBLB double knock out (×s; 322.69).

FIG. 30A-B: Effector function of double knock out three-stage GM NK against HL60 in the (30A) presence or (30B) absence of TGFβ treatment.

FIG. 31A-B: Effector function of double knock out three-stage GM NK against K562 in the (31A) presence or (31B) absence of TGFβ treatment.

FIG. 32A-E: (32A) GM-CSF, (32B) sCD137, (32C) IFN-γ, (32D) TNF-α, and (32E) perform secretion of NK cells in the presence or absence of TGFβ treatment, and in the presence of K562, HL60, RPMI, or KG1 cells. Bars from left to right indicate secretion for the mock transfected, TGFBR2 knock out, CBLB knockout and the TGFBR2/CBLB double knockout.

FIG. 33 shows the CD16 transduction efficiency. Transduction of CD34 cells were optimized testing various conditions. The lentiviral transduction was optimized at 1× transduction at 100 MOI on day 5 at 600 g to achieve a median transduction efficiency over 70% (43-81% for cells obtained from eight different donors (#92-#99).

FIG. 34 shows the PNK-CD16VP expansion results for cells obtained from eight different donors (#92-#99).

FIG. 35 shows PNK-CD16CP phenotype post expansion data for cells obtained from eight different donors (#92-#99).

FIG. 36 shows PNK-CD16VP construct validation data for cells obtained from eight different donors (#92-#99). As shown in the left panel, the top line is the data for CD16VP the bottom line is for PNK-NT. In the bar graphs of the right panel, the order of the 6 bars for the activation by PMA are: untreated, PMA treated, PMA+a−TACE D1 (A12) untreated, PMA treated and PMA+a−TACE D1 (A12). In the bar graphs showing the data for activation by ADCC, the order of the 6 bars are Daudi Uncoated, Daudi+IgG, Daudi+a−CD38, Daudi Uncoated, Daudi+IgG and Daudi+a−CD38.

FIG. 37 shows data showing PNK-CD16VP ADCC function for cells obtained from eight different donors (#92-#99). As shown, PNK-CD16VP exhibited improvement in ADCC against Daudi with CD20, CD38 and CD319.

### V. TERMINOLOGY

In the description that follows, the terms should be given their plain and ordinary meaning when read in light of the specification. One of skill in the art would understand the terms as used in view of the whole specification.

As used herein, the terms “immunomodulatory compound” and “IMiD™” do not encompass thalidomide.

“Genetically modify” has its plain and ordinary meaning when read in light of the specification, and may include but is not limited to, for example, a process for modifying an organism or a cell such as a bacterium, a lymphocyte such as a T-cell or NK cell, bacterial cell, eukaryotic cell, insect, plant or mammal with genetic material, such as nucleic acid, that has been altered using genetic engineering techniques. For example, nucleic acid such as DNA can be inserted in the host genome by first isolating and copying the genetic material of interest using molecular cloning methods to generate a DNA sequence, or by synthesizing the DNA, and then inserting this construct into the host organism. Genes and gene expression can also be removed, or “knocked out”, using gene editing. Those of skill in the art can appreciate the many techniques for knocking out genes. Without being limiting, genes and/or gene expression may be knocked out with techniques using RNA interference, CRISPRs or TALENs, for example. Gene targeting is a different technique that uses homologous recombination to change an endogenous gene, and can be used to delete a gene, remove exons, add a gene, or introduce point mutations.

Genetic modification performed by transduction is described herein. “Transduction” has its plain and ordinary meaning when read in light of the specification, and may include but is not limited to, for example, methods of transferring genetic material, such as, for example, DNA or RNA, to a cell by way of a vector. Common techniques use viral vectors, electroporation, and chemical reagents to increase cell permeability. The DNA can be transferred by a virus, or via a viral vector. As described herein, methods are provided for modifying immune cells, e.g., natural killer cells. Viral vectors may be derived from adenovirus, adeno-associated virus (AAV), retroviruses and lentiviruses.

Various transduction techniques have been developed, which utilize recombinant infectious virus particles for delivery. This represents a currently preferred approach to the transduction of cells. Viral vectors that may be used for transduction can include virus vectors derived from simian virus 40, adenoviruses, adeno-associated virus (AAV), lentiviral vectors, and retroviruses. Thus, gene transfer and expression methods are numerous but essentially function to introduce and express genetic material in mammalian cells. Several of the above techniques can be used to transduce cells, including calcium phosphate transfection, protoplast fusion, electroporation, and infection with recombinant adenovirus, adeno-associated virus, lentivirus, or retrovirus vectors. Lymphocytes have been successfully transduced by electroporation and by retroviral or lentiviral infection. As such, retroviral and lentiviral vectors can provide a highly efficient method for gene transfer in eukaryotic cells. Retroviral and lentiviral vectors provide highly efficient methods for gene transfer into lymphocytes such as T-cells and NK cells. Moreover, retroviral or lentiviral integration takes place in a controlled fashion and results in the stable integration of one or a few copies of the new genetic information per cell.

“Gene editing,” has its plain and ordinary meaning when read in light of the specification, and may include but is not limited to, for example, a type of genetic engineering in which DNA is inserted, deleted or replaced in the genome of a living organism using a nuclease or an engineered nuclease or nucleases. Without being limiting, the nuclease can be of the CRISPR/CAS9 system, a zinc finger nuclease or TALEN nuclease. The nuclease can be used to target a locus, or a targeted locus on a nucleic acid sequence.

“TALEN” or “Transcription activator-like effector nuclease” has its plain and ordinary meaning when read in light of the specification, and may include but is not limited to, for example, restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind practically any desired DNA sequence, so when combined with a nuclease, DNA can be cut at specific locations. The restriction enzymes can be introduced into cells, for use in gene editing or for genome editing in situ, a technique known as genome editing with engineered nucleases. Alongside zinc finger nucleases and CRISPR/Cas9, TALEN is a prominent tool in the field of genome editing. These nucleases may be used for “knocking out” genes.

“CRISPRs” (clustered regularly interspaced short palindromic repeats), has its plain and ordinary meaning when read in light of the specification, and may include but is not limited to, for example, are segments of prokaryotic DNA containing short repetitions of base sequences. Each repetition is followed by short segments of “spacer DNA” from previous exposures to a bacterial virus or plasmid. The CRISPR/Cas system is a prokaryotic immune system that confers resistance to foreign genetic elements such as plasmids and phages and provides a form of acquired immunity. CRISPR spacers recognize and cut these exogenous genetic elements in a manner analogous to RNAi in eukaryotic organisms. CRISPR/Cas system has been used for gene editing (adding, disrupting or changing the sequence of specific genes) and gene regulation in species throughout the tree of life. By delivering the Cas9 protein and appropriate guide RNAs into a cell, the organism's genome can be cut at any desired location. One of skill in the art may appreciate the use of CRISPR to build RNA-guided gene editing tools capable of altering the genomes of entire populations.

“Lenalidomide” has its plain and ordinary meaning when read in light of the specification, and may include but is not limited to, for example, 3-(4′aminoisoindoline-1′-one)-1-piperidine-2,6-dione (Chemical Abstracts Service name) or 2,6-Piperidinedione, 3-(4-amino-1,3-dihydro-1-oxo-2H-isoindol-2-yl)- (International Union of Pure and Applied Chemistry (IUPAC) name). As used herein, “pomalidomide” means 4-amino-2-(2,6-dioxopiperidin-3-yl)isoindole-1,3-dione.

“Multipotent,” has its plain and ordinary meaning when read in light of the specification, and may include but is not limited to, for example, when referring to a cell, means that the cell has the capacity to differentiate into a cell of another cell type. In certain alternatives, “a multipotent cell” is a cell that has the capacity to grow into a subset of the mammalian body's approximately 260 cell types. Unlike a pluripotent cell, a multipotent cell does not have the capacity to form all of the cell types.

“Feeder cells” has its plain and ordinary meaning when read in light of the specification, and may include but is not limited to, for example, cells of one type that are co-cultured with cells of a second type, to provide an environment in which the cells of the second type can be maintained, and perhaps proliferate. Without being bound by any theory, feeder cells can provide, for example, peptides, polypeptides, electrical signals, organic molecules (e.g., steroids), nucleic acid molecules, growth factors (e.g., bFGF), other factors (e.g., cytokines), and metabolic nutrients to target cells. In certain alternatives, feeder cells grow in a mono-layer.

“Natural killer cells” or “NK cells,” has its plain and ordinary meaning when read in light of the specification, and may include but is not limited to, for example, natural killer cells from any tissue source and also includes natural killer cells produced using methods such as those described herein.

“Placental perfusate” has its plain and ordinary meaning when read in light of the specification, and may include but is not limited to, for example, perfusion solution that has been passed through at least part of a placenta, e.g., a human placenta, e.g., through the placental vasculature, and includes a plurality of cells collected by the perfusion solution during passage through the placenta.

“Placental perfusate cells” has its plain and ordinary meaning when read in light of the specification, and may include but is not limited to, for example, nucleated cells, e.g., total nucleated cells, isolated from, or isolatable from, placental perfusate.

“Tumor cell suppression,” “suppression of tumor cell proliferation,” and the like, has their plain and ordinary meaning when read in light of the specification, and may include but is not limited to, for example, slowing the growth of a population of tumor cells, e.g., by killing one or more of the tumor cells in said population of tumor cells, for example, by contacting or bringing, e.g., NK cells or an NK cell population produced using a three-stage method described herein into proximity with the population of tumor cells, e.g., contacting the population of tumor cells with NK cells or an NK cell population produced using a three-stage method described herein. In certain alternatives, said contacting takes place in vitro. In other alternatives, said contacting takes place in vivo.

“Hematopoietic cells” has its plain and ordinary meaning when read in light of the specification, and may include but is not limited to, for example, hematopoietic stem cells and hematopoietic progenitor cells.

“CBLB,” E3 ubiquitin ligase (casitas B-lineage lymphoma-b), is a negative regulator of T-cell activation. In some alternatives described herein, a population of cells comprising natural killer cells is provided, wherein the natural killer (NK) cells are genetically modified such that they lack expression of an NK inhibitory molecule or manifest a reduced expression of an NK inhibitory molecule. In some alternatives, the NK inhibitory molecules is a negative regulator of T-cell activation. In some alternatives, the NK inhibitory molecule is CBLB.

“NKG2A” is a form of a C-type lectin receptor, which are expressed predominantly on the surface of NK cells and a subset of CD8⁺ T-lymphocyte. These receptors stimulate or inhibit cytotoxic activity of NK cells, therefore they are divided into activating and inhibitory receptors according to their function. In some alternatives described herein, a population of cells comprising natural killer cells is provided, wherein the natural killer (NK) cells are genetically modified such that they lack expression of an NK inhibitory molecule or manifest a reduced expression of an NK inhibitory molecule. In some alternatives, the NK inhibitory molecules is a form of a C-type lectin receptor. In some alternatives, the NK inhibitory molecule is NKG2A. In some alternatives herein, NKG2A knockout NK cells have up to a three-fold or more increase in cytotoxicity in comparison to untreated cells that have no NKG2A knockout, such as naturally occurring NK cells.

“TGFBR2” is a TGF beta receptor. In some alternatives described herein, a population of cells comprising natural killer cells is provided, wherein the natural killer (NK) cells are genetically modified such that they lack expression of an NK inhibitory molecule or manifest a reduced expression of an NK inhibitory molecule. In some alternatives, this NK inhibitory molecule is TGFBR2.

“CD16” is a low affinity Fc receptor found on the surface of immune cells, e.g., natural killer cells, neutrophil polymorphonuclear leukocytes, monocytes and macrophages.

“ADAM metallopeptidase domain 17” (ADAM 17), also known as TACE, is an enzyme that belongs to the ADAM protein family of disintegrins and metalloproteases. ADAM may be involved in the processing of TNF-α. In some alternatives herein, a population of cells comprising natural killer cells is provided, wherein the natural killer (NK) cells are genetically modified to comprise a modified or mutant CD16. In some alternatives, the modified or mutant CD16 is resistant to ADAM17 cleavage.

“Drug selection markers,” has its plain and ordinary meaning when read in light of the specification, and may include a selection marker to facilitate identification or selection of host cells that have received a vector and have the selection marker. Without being limiting, selection markers may include genes encoding proteins conferring resistance to a selection agent, e.g., PuroR gene, ZeoR gene, HygroR gene, neoR gene, and/or the blasticidin resistance gene,

The “undefined component” has its plain and ordinary meaning when read in light of the specification, and may include but is not limited to, for example, components whose constituents are not generally provided or quantified. Examples of an “undefined component” include, without limitation, serum, for example, human serum (e.g., human serum AB) and fetal serum (e.g., fetal bovine serum or fetal calf serum).

As used herein, “+”, when used to indicate the presence of a particular cellular marker, means that the cellular marker is detectably present in fluorescence activated cell sorting over an isotype control; or is detectable above background in quantitative or semi-quantitative RT-PCR.

As used herein, “−”, when used to indicate the presence of a particular cellular marker, means that the cellular marker is not detectably present in fluorescence activated cell sorting over an isotype control; or is not detectable above background in quantitative or semi-quantitative RT-PCR.

“Placental derived NK cells” or pNK cells has its plain and ordinary meaning when read in light of the specification, and may include NK cells derived from the postpartum placenta and umbilical cord. Prior to processing of the pNK cells, donor eligibility is done by a series of test, such as serology, bacteriology and HLA typing. Isolation is performed under sterile conditions by those of skill in the art.

“Expressed,” has its plain and ordinary meaning when read in light of the specification, and may include but is not limited to, for example, for indicating the presence of a particular cellular marker, means that the cellular marker is detectably present or is detectably present above background, using a technique to detect the presence of a protein or nucleic acid known to one of skill in the art. As used herein, “not expressed,” or “lacks expression,” and the like, when used to indicate the presence of a particular cellular marker, means that the cellular marker is not detectably present or is not detectable above background, using a technique to detect the presence of a protein or nucleic acid known to one of skill in the art.

As used herein, “lacks function,” “does not function,” and the like, when used to indicate the presence of a particular function, means the function is not detectably present or is not detectable above background, using a standard assay to detect said function known to one of skill in the art.

### VI. DETAILED DESCRIPTION

In spite of the advantageous properties of NK cells in killing tumor cells and virus-infected cells, there remains a need in the art to develop efficient methods to produce and expand natural killer cells that retain tumoricidal functions.

NK cells are innate lymphoid cells (ILCs). Innate lymphoid cells are related through their dependency on transcription factor ID2 for development.

Provided herein are populations of genetically modified (GM) natural killer (NK) cells, methods of producing populations of GM NK cells, and methods of using GM NK cells.

1. GM NK Cells with Altered Expression of NK Inhibitory Molecules

In certain alternatives, GM NK cells provided herein lack expression and/or function of CBLB, NKG2A and/or TGFBR2 or show reduced expression and/or function of CBLB, NKG2A and/or TGFBR2, as compared to naturally occurring NK cells or unmodified NK cells controls. Gene sequences for CBLB, NKG2A, and TGFBR2 are known by those of skill in the art and exemplary sequences are described herein. Standard techniques known to those of skill in the art can be used to modify the sequences described herein.

CBLB (Casitas B-lineage lymphoma proto-oncogene B) is an intracellular protein that acts downstream of RTK, CD28, CTLA4, and TGFb signaling pathways, and maintains a balance between immunity and tolerance. GenBank™ accession number Q13191.2 provides an exemplary human CBLB amino acid sequence. GenBank™ accession number NM_001321788.1 provides an exemplary human CBLB nucleotide sequence. Without wishing to be bound by any particular mechanism or theory, it is hypothesized that knocking out CBLB in NK cells will lower the NK cell activation threshold, rendering NK cells hyperactive. In certain alternatives, provided herein are populations of GM NK cells lacking expression of CBLB. In certain alternatives, provided herein are populations of GM NK cells having a reduced expression of CBLB. In certain alternatives, the GM NK cells are human GM NK cells. In certain alternatives, provided herein are populations of GM NK cells, wherein CBLB expression has been knocked out. Genes may be knocked out with techniques using RNA interference, CRISPRs or TALENs. In specific alternatives, the knockout of CBLB expression is performed by a CRISPR-related technique. In certain alternatives, the knockout of CBLB expression generates a population of NK cells having higher cytotoxicity against tumor cells than NK cells without a CBLB knockout, e.g., unmodified NK cells or naturally occurring NK cells. In specific alternatives, the tumor cells are multiple myeloma cells. In specific alternatives, the tumor cells are RPMI8226 cells. In specific alternatives, the tumor cells are U266 cells. In specific alternatives, the tumor cells are ARH77 cells. In certain alternatives, the knockout of CBLB expression generates a population of NK cells having higher IFNγ secretion than NK cells without a CBLB knockout e.g., naturally occurring or unmodified NK cells. In certain alternatives, the knockout of CBLB expression generates a population of NK cells having higher degranulation than NK cells without a CBLB knockout e.g., naturally occurring NK cells or unmodified NK cells. In specific alternatives, the higher degranulation is measured by an increase in CD107a. Measurement techniques of markers of an immune response is known to those of skill in the art. CD107a may be measured by flow cytometry based methods, using an anti-CD107a antibody, for example. In certain alternatives, the knockout of CBLB expression results in a change in the secretion of one or more of GM-CSF, soluble CD137 (sCD137), IFNγ, MIP1α, MIP1β, TNFα or perform in NK cells, as compared to NK cells without a CBLB knockout, such as naturally occurring or unmodified NK cells. In certain alternatives, the knockout of CBLB expression results in a change in the secretion concentrations of one or more of GM-CSF, soluble CD137 (sCD137), IFNγ, MIP1α, MIP1β, TNFα or perform in NK cells, as compared to NK cells without a CBLB knockout e.g., naturally occurring or unmodified NK cells.

NKG2A is a protein that binds to CD94 in NK cells and inhibits NK activity. GenBank™ accession number AAL65234.1 provides an exemplary human NKG2A amino acid sequence. GenBank™ accession number AF461812.1 provides an exemplary human NKG2A nucleotide sequence. Without wishing to be bound by any particular mechanism or theory, it is hypothesized that generation of a NKG2A deficient, functionally mature NK cell product will provide an enhanced therapeutic activity. In certain alternatives, provided herein are populations of GM NK cells lacking expression of NKG2A. In certain alternatives, the GM NK cells are human GM NK cells. In certain alternatives, the populations of GM NK cells have a reduced expression of NKG2A. Certain alternatives, provided herein concern populations of GM NK cells, wherein NKG2A expression has been knocked out. Genes may be knocked out with techniques using RNA interference, CRISPRs or TALENs. In specific alternatives, the knockout of NKG2A expression is performed by a CRISPR-related technique. In certain alternatives, the knockout of NKG2A expression generates a population of NK cells having a higher cytotoxicity against tumor cells than NK cells without a NKG2A knockout e.g., unmodified NK cells or naturally occurring NK cells. In specific alternatives, the tumor cells are multiple myeloma cells. In specific alternatives, the tumor cells are RPMI8226 cells. In specific alternatives, the tumor cells are U266 cells. In specific alternatives, the tumor cells are ARH77 cells. In certain alternatives, the knockout of NKG2A expression generates a population of NK cells having a higher IFNγ secretion than NK cells without a NKG2A knockout, e.g., unmodified NK cells or naturally occurring NK cells. In certain alternatives, the knockout of NKG2A expression generates a population of NK cells having a higher degranulation than NK cells without a NKG2A knockout e.g., unmodified NK cells or naturally occurring NK cells. In specific alternatives, the higher degranulation is measured by an increase in CD107a detection. In certain alternatives, the knockout of NKG2A expression results in a change in the secretion of one or more of GM-CSF, sCD137, IFNγ, MIP1α, MIP1β, TNFα and/or perform in NK cells, compared with NK cells without a NKG2A knockout e.g., unmodified NK cells or naturally occurring NK cells. In certain alternatives, the knockout of CBLB expression results in a change in the secretion concentrations of one or more of GM-CSF, soluble CD137 (sCD137), IFNγ, MIP1α, MIP1β, TNFα and/or perform in NK cells, compared to NK cells without a CBLB knockout e.g., unmodified NK cells or naturally occurring NK cells. In some alternatives herein, NKG2A knockout NK cells have up to a three-fold or more increase in cytotoxicity in comparison to untreated cells that have no NKG2A knockout.

TGF-β1 is a potent immunosuppressor that promotes evasion from NK cell anti-tumor immunity. TGFβ signaling acts through TGFβ type 2 receptor 2 (TGFBR2 or TβRII), and controls expression of hundreds of genes downstream. Downstream events include Smad2/3 phosphorylation and downregulation of NK activating receptors. GenBank™ accession number ABG65632.1 provides an exemplary human TGFBR2 amino acid sequence. GenBank™ accession number KU178360.1 provides an exemplary human TGFBR2 nucleotide sequence. Accordingly, without wishing to be bound by any particular mechanism or theory, it is hypothesized that generation of TGFBR2 knockouts in NK cells provides a population of NK cells having a greater effector function and higher expression of activating receptors. Certain alternatives provided herein comprise populations of GM NK cells lacking expression of TGFBR2. In certain alternatives, provided herein populations of GM NK cells have a reduced expression of TGFBR2. In certain alternatives, provided herein are populations of GM NK cells, wherein TGFBR2 expression has been knocked out. Genes may be knocked out with techniques using RNA interference, CRISPRs or TALENs. In certain alternatives, the GM NK cells are human GM NK cells. In specific alternatives, the knockout of TGFBR2 expression is performed by a CRISPR-related technique. In certain alternatives, the knockout of TGFBR2 expression generates a population of NK cells having NK cells with a higher cytotoxicity against tumor cells than NK cells without a TGFBR2 knockout, such as unmodified NK cells or naturally occurring NK cells. In specific alternatives, the tumor cells are multiple myeloma cells. In specific alternatives, the tumor cells are chronic myeloid leukemia cells. In specific alternatives, the tumor cells are acute myeloid leukemia cells. In specific alternatives, the tumor cells are RPMI8226 cells. In specific alternatives, the tumor cells are U266 cells. In specific alternatives, the tumor cells are K562 cells. In specific alternatives, the tumor cells are HL-60 cells. In specific alternatives, the tumor cells are ARH77 cells. In certain alternatives, the knockout of TGFBR2 expression results in NK cells with higher IFNγ secretion than NK cells without a TGFBR2 knockout. In certain alternatives, the knockout of TGFBR2 expression generates a population of NK cells having a higher degranulation than NK cells without a TGFBR2 knockout e.g., unmodified NK cells or naturally occurring NK cells. In specific alternatives, the higher degranulation is measured by an increase in CD107a detection. Measurement techniques of markers of an immune response is known to those of skill in the art. CD107a may be measured by flow cytometry based methods, using an anti-CD107a antibody, for example. In certain alternatives, the knockout of TGFBR2 expression results in a change in the secretion of one or more of GM-CSF, sCD137, IFNγ, MIP1α, MIP1β, TNFα and/or perform in NK cells, compared with NK cells without a TGFBR2 knockout e.g., unmodified NK cells or naturally occurring NK cells. In certain alternatives, the knockout of CBLB expression results in a change in the secretion concentrations of one or more of GM-CSF, soluble CD137 (sCD137), IFNγ, MIP1α, MIP1β, TNFα and/or perform in NK cells, compared to NK cells without a CBLB knockout, such as unmodified NK cells or naturally occurring NK cells. In certain alternatives, the knockout of TGFBR2 expression results in reduced levels of Smad2/3 phosphorylation, compared with NK cells without a TGFBR2 knockout, e.g., unmodified NK cells or naturally occurring NK cells. In certain alternatives, the knockout of TGFBR2 expression results in increased levels of Smad2/3 phosphorylation, compared with NK cells without a TGFBR2 knockout e.g., unmodified NK cells or naturally occurring NK cells. In certain alternatives, the knockout of TGFBR2 expression results in increased expression of one or more of DNAM-1, NKG2D and/or NKp30.

2. GM NK Cells Comprising Modified CD16

Gene sequences for CD16 are known by those of skill in the art and exemplary sequences are described herein. Standard techniques known to those of skill in the art can be used to modify the sequences described herein.

CD16 (cluster of differentiation 16) consists of two isoforms, the Fc receptors, FcγRIIIa and FcγRIIIb, also known as CD16a and CD16b, respectively. CD16a is found on natural killer cells. CD16 binds to the Fc portion of IgG antibodies, which activates the natural killer cell for antibody-dependent cell-mediated cytotoxicity (ADCC). CD16a and CD16b both contain cleavage sites targeted by ADAM17. Proteolytic cleavage of CD16a by ADAM17 occurs upon NK cell activation, and leads to soluble CD16 release into the plasma. GenBank™ accession number NP_000560.6 provides an exemplary human wildtype CD16a amino acid sequence. GenBank™ accession number BC036723.1 provides an exemplary human wildtype CD16a nucleotide sequence.

In certain alternatives, provided herein are GM NK cells comprising modified CD16. In certain alternatives, the GM NK cells are human GM NK cells. In certain alternatives, the modified CD16 is modified human CD16. In specific alternatives, the modified CD16 has a higher affinity for IgG than wildtype CD16. In more specific alternatives, the modified CD16 has a valine (Val or V) at position 158 of CD16a. In specific alternatives, the modified CD16 is resistant to ADAM17 cleavage. In more specific alternatives, the CD16 has a proline (Pro or P) at position 197 (an S197P mutation) in CD16a. In certain alternatives, the modified CD16 has a higher affinity for IgG than wildtype CD16 and is resistant to ADAM17 cleavage. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In certain alternatives, the CD16 has a valine at position 158 of CD16a and a proline at position 197 of CD16a. In certain alternatives, the modified CD16 contains an IgK signal peptide. In certain alternatives, the modified CD16 contains a CD16 signal peptide. In certain alternatives, the modified CD16 is introduced into the NK cells via viral infection. In certain alternatives, the modified CD16 is introduced into hematopoietic cells via viral infection, which hematopoietic cells are then differentiated into NK cells. In certain alternatives, the modified CD16 is introduced via a lentiviral vector. In certain alternatives, the lentiviral vector has either a CMV or a EF1α promoter. In certain alternatives, the lentiviral vector comprises one or more drug selection markers. In certain alternatives, the modified CD16 is introduced via a retroviral vector. In certain alternatives, the retroviral vector comprises one or more drug selection markers.

In certain alternatives, the GM-NK cells with a modified CD16 disclosed herein show improved antibody-dependent cellular cytotoxicity (ADCC) than NK cells with wildtype CD16, such as naturally occurring NK cells.

3. GM NK Cells Comprising Genetic Modifications

In certain alternatives, GM NK cells provided herein (1) lack expression and/or function of CBLB, NKG2A and/or TGFBR2 or show reduced expression and/or function of CBLB, NKG2A and/or TGFBR2, and/or (2) comprise a modified CD16 described herein. In a specific alternative, GM NK cells provided herein lack expression and/or function of CBLB and TGFBR2.

4. Production of GM NK Cells and GM NK Cell Populations

In certain alternatives, production of GM NK cells and/or GM NK cell populations by the present methods comprises expanding a population of hematopoietic cells. In certain alternatives, NK cells are genetically modified on day 1, 2, 3, 4, 5, 6, 7, 8, 9, 10, 11, 12, 13, 14, 15, 16, 17, 18, 19, 20, or 21 of the 35-day, three-stage process for producing NK cells, as described herein and in International Patent Application Publication No. WO 2016/109661, which is incorporated by reference herein in its entirety. In certain alternatives, NK cells are genetically modified on day 1, 2, 3, 4, 5, 6, 7, 8, 9, 10, 11, 12, 13, 14, 15, 16, 17, 18, 19, 20, or 21 of the 35-day, three-stage process for producing NK cells or any day in between a range defined by any two of the aforementioned days. In certain alternatives, NK cells are genetically modified on day 3, 5, 7, or 9 of the 35-day, three-stage process for producing NK cells. In certain alternatives, NK cells are genetically modified on day 3, 5, 7, or 9 of the 35-day, three-stage process for producing NK cells or any day in between a range defined by any two aforementioned days. In certain alternatives, NK cells are genetically modified on day 5 of the 35-day, three-stage process for producing NK cells. In certain alternatives, NK cells are genetically modified on day 3 of the 35-day, three-stage process for producing NK cells. In certain alternatives, NK cells are genetically modified on day 7 of the 35-day, three-stage process for producing NK cells. In certain alternatives, NK cells are genetically modified on day 9 of the 35-day, three-stage process for producing NK cells. In certain alternatives, genetic modification comprises knockout of CBLB, NKG2A and/or TGFBR2 as described herein. In certain alternatives, genetic modification comprises knockout of CBLB, NKG2A and/or TGFBR2 as described herein. In certain alternatives, genetic modification comprises introduction of a modified CD16 as described herein.

Gene modification by knockout as described herein may be done by any method known to one of skill in the art. For example, knockout may be done by a gene editing technique. Genes may be knocked out with techniques using RNA interference, CRISPRs or TALENs. In certain alternatives, the gene editing technique is a CRISPR-related technique. In certain alternatives, the gene editing technique is a meganuclease-related technique. In certain alternatives, the gene editing technique is a zinc finger nuclease (ZFN)-related technique. In certain alternatives, the gene editing technique is a transcription activator-like effector-based nuclease (TALEN)-related technique.

In specific alternatives, the CRISPR-related technique involves a CRISPR/Cas9 system. For example, to produce a knockout using a CRISPR/Cas9 system, Crispr guide RNAs (gRNAs) can be chemically modified and synthesized in single-guide (sgRNA) format. Cas9 may then be delivered as mRNA with pseudouridine (T) modification. A nucleofector can then be utilized to deliver sgRNA and Cas9 mRNA to the cells.

Introduction of a modified gene as described herein may be done by any method known to one of skill in the art. For example, genetically modified genes may be introduced via a retroviral vector. In some alternatives, the genetically modified genes are introduced via a lentiviral vector.

During cell expansion, for example, in the three-stage method for producing NK cells, a plurality of hematopoietic cells within the hematopoietic cell population differentiate into NK cells. During this process, said NK cells are genetically modified such that the resultant NK cells are GM NK cells. In certain alternatives, the genetic modifications are performed before the cells differentiate into NK cells. In certain alternatives, the genetic modifications are performed after the cells differentiate into NK cells. In certain alternatives, the genetic modifications are performed on NK progenitor cells. In one aspect, provided herein is a method of producing GM NK cells comprising producing NK cells by a method comprising culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and IL-15, and lacking a stem cell mobilizing agent and LMWH, to produce a third population of cells, wherein the third population of cells comprises natural killer cells that are CD56⁺, CD3⁻, and wherein at least 70%, for example at least 80%, 85%, 90%, 95% or a percentage that falls within a range defined by any two of the aforementioned percentages, of the natural killer cells are viable. In certain alternatives, such natural killer cells comprise natural killer cells that are CD16⁻. In certain alternatives, such natural killer cells comprise natural killer cells that are CD94⁺. In certain alternatives, such natural killer cells comprise natural killer cells that are CD94⁺ or CD16⁺. In certain alternatives, such natural killer cells comprise natural killer cells that are CD94⁻ or CD16⁻. In certain alternatives, such natural killer cells comprise natural killer cells that are CD94⁺ and CD16⁺. In certain alternatives, such natural killer cells comprise natural killer cells that are CD94⁻ and CD16⁻. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In one aspect, provided herein is a method of producing GM NK cells comprising producing NK cells by a method comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56⁺, CD3⁻, and CD11a⁺. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In one aspect, provided herein is a method of producing GM NK cells comprising producing NK cells by a method comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of stem cell factor (SCF) and LMWH, to produce a third population of cells; wherein the third population of cells comprises natural killer cells that are CD56⁺, CD3⁻, and CD11a⁺. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In one aspect, provided herein is a method of producing GM NK cells comprising producing NK cells by a method comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking each of SCF, a stem cell mobilizing agent, and LMWH, to produce a third population of cells; wherein the third population of cells comprises natural killer cells that are CD56⁺, CD3⁻, and CD11a⁺. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In one aspect, provided herein is a method of producing GM NK cells comprising producing NK cells by a method comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and IL-15, and lacking each of a stem cell mobilizing agent and LMWH, to produce a third population of cells; and (d) isolating CD11a⁺ cells from the third population of cells to produce a fourth population of cells; wherein the fourth population of cells comprises natural killer cells that are CD56⁺, CD3⁻, and CD11a⁺. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In certain alternatives, of any of the above alternatives, said natural killer cells express perform and/or EOMES. In certain alternatives, said natural killer cells do not express either RORγt and/or IL1R1.

GM NK cells described herein may be produced from any type of NK cells, or via any production method for producing NK cells. GM NK cells described herein may be isolated or produced using methods described herein. In certain alternatives, NK cells produced using methods herein are modified after production to produce GM NK cells. In certain alternatives, NK cells produced using the methods herein are modified during production to produce GM NK cells. In certain alternatives, NK cells produced using the methods herein are modified before production, in order to produce GM NK cells. GM NK cells herein refer to the cells to which the genetic modifications were made directly, and to any progeny of such cells comprising the genetic modifications. In certain alternatives, GM NK cells provided herein are produced via a three-stage method, e.g., a three-stage method as described in International Patent Publication No. WO 2016/109661, which is incorporated by reference herein in its entirety. In certain alternatives, GM NK cells provided herein are produced from placental NK cells, for example, placental NK cells as described in U.S. Pat. No. 8,263,065, U.S. Patent Application Publication No. 2011/0280849, and/or U.S. Patent Application Publication No. 2015/0366910, each of which are incorporated by reference herein in their entirety. In certain alternatives, GM NK cells provided herein are produced by a two-step or three-step method as described in U.S. Pat. No. 8,926,964 and/or U.S. Publication No. 2015/0225697, each of which is incorporated by reference herein in its entirety. In certain alternatives, GM NK cells provided herein are produced via any of the methods described in International Patent Publication No. WO 2016/109668, which is incorporated by reference herein in its entirety.

a. 6.4.1 Production of GM NK Cell Populations Using a Three-Stage Method

In one alternative, GM NK cells provided herein are produced via a three-stage method, e.g., a three-stage method as described in International Patent Publication No. WO 2016/109661, which is incorporated by reference herein in its entirety. In certain alternatives, genetic modifications are introduced into the NK cells during the first, second, and/or third stage. In certain alternatives, genetic modifications are introduced into the NK cells during the first and second stage. In certain alternatives, genetic modifications are introduced into the NK cells during the first and third stage. In certain alternatives, genetic modifications are introduced into the NK cells during the second and third stage. In certain alternatives, genetic modifications are introduced into the NK cells during the first stage. In certain alternatives, genetic modifications are introduced into the NK cells during the second stage. In certain alternatives, genetic modifications are introduced into the NK cells during the third stage.

In a certain alternative, the three-stage method comprises a first stage (“stage 1”) comprising culturing hematopoietic stem cells or progenitor cells, e.g., CD34⁺ stem cells or progenitor cells, in a first medium for a specified time period, e.g., as described herein, to produce a first population of cells. In certain alternatives, the first medium comprises a stem cell mobilizing agent and thrombopoietin (Tpo). In certain alternatives, the first medium comprises in addition to a stem cell mobilizing agent and Tpo, one or more of LMWH, Flt-3L, SCF, IL-6, IL-7, G-CSF, and/or GM-CSF. In a specific alternative, the first medium comprises each of the first medium comprises in addition to a stem cell mobilizing agent and Tpo, each of LMWH, Flt-3L, SCF, IL-6, IL-7, G-CSF, and/or GM-CSF. In a specific alternative, the first medium lacks added LMWH. In a specific alternative, the first medium lacks added desulphated glycosaminoglycans. In a specific alternative, the first medium lacks LMWH. In a specific alternative, the first medium lacks desulphated glycosaminoglycans. In a specific alternative, the first medium comprises each of the first medium comprises in addition to a stem cell mobilizing agent and Tpo, each of Flt-3L, SCF, IL-6, IL-7, G-CSF, and/or GM-CSF. In specific alternatives, the first medium lacks leukemia inhibiting factor (LIF), macrophage inhibitory protein-1alpha (MIP-1α) or both.

In certain alternatives, subsequently, in “stage 2” said cells are cultured in a second medium for a specified time period, e.g., as described herein, to produce a second population of cells. In certain alternatives, the second medium comprises a stem cell mobilizing agent and interleukin-15 (IL-15), and lacks Tpo. In certain alternatives, the second medium comprises, in addition to a stem cell mobilizing agent and IL-15, one or more of LMWH, Flt-3, SCF, IL-6, IL-7, G-CSF, and/or GM-CSF. In certain alternatives, the second medium comprises, in addition to a stem cell mobilizing agent and IL-15, each of LMWH, Flt-3, SCF, IL-6, IL-7, G-CSF, and/or GM-CSF. In a specific alternative, the second medium lacks added LMWH. In a specific alternative, the second medium lacks added desulphated glycosaminoglycans. In a specific alternative, the second medium lacks heparin, e.g., LMWH. In a specific alternative, the second medium lacks desulphated glycosaminoglycans. In certain alternatives, the second medium comprises, in addition to a stem cell mobilizing agent and IL-15, each of Flt-3, SCF, IL-6, IL-7, G-CSF, and/or GM-CSF. In specific alternatives, the second medium lacks leukemia inhibiting factor (LIF), macrophage inhibitory protein-1 alpha (MIP-1α) or both.

In certain alternatives, subsequently, in “stage 3” said cells are cultured in a third medium for a specified time period, e.g., as described herein, to produce a third population of cell, e.g., natural killer cells. In certain alternatives, the third medium comprises IL-2 and/or IL-15, and lacks a stem cell mobilizing agent and/or LMWH. In certain alternatives, the third medium comprises in addition to IL-2 and/or IL-15, one or more of SCF, IL-6, IL-7, G-CSF, and/or GM-CSF. In certain alternatives, the third medium comprises, in addition to IL-2 and/or IL-15, each of SCF, IL-6, IL-7, G-CSF, and/or GM-CSF. In specific alternatives, the first medium lacks one, two, or all three of LIF, MIP-1α, and/or Flt3L. In specific alternatives, the third medium lacks added desulphated glycosaminoglycans. In specific alternatives, the third medium lacks desulphated glycosaminoglycans. In specific alternatives, the third medium lacks heparin, e.g., LMWH.

In a specific alternative, the three-stage method is used to produce NK cell populations. In certain alternatives, the three-stage method is conducted in the absence of stromal feeder cell support. In certain alternatives, the three-stage method is conducted in the absence of exogenously added steroids (e.g., cortisone, hydrocortisone, or derivatives thereof).

In certain aspects, the three-stage method produces natural killer cells that comprise at least 20% CD56⁺CD3⁻ natural killer cells. In certain aspects, the three-stage method produces natural killer cells that comprise at least 40% CD56⁺CD3⁻ natural killer cells. In certain aspects, the three-stage method produces natural killer cells that comprise at least 60% CD56⁺CD3⁻ natural killer cells. In certain aspects, the three-stage method produces natural killer cells that comprise at least 70% CD56⁺CD3⁻ natural killer cells. In certain aspects, the three-stage method produces natural killer cells that comprise at least 80% CD56⁺CD3⁻ natural killer cells. In certain aspects, the three-stage method produces natural killer cells that comprise at least 20%, 30%, 40%, 50%, 60%, 70% or 80% CD56⁺CD3⁻ natural killer cells or a percent in between a range defined by any two of the aforementioned percentages.

In certain aspects, the three-stage method disclosed herein produces natural killer cells that comprise at least 20% CD56⁺CD3⁻CD11a⁺ natural killer cells. In certain aspects, the three-stage method disclosed herein produces natural killer cells that comprise at least 40% CD56⁺CD3⁻CD11a⁺ natural killer cells. In certain aspects, the three-stage method disclosed herein produces natural killer cells that comprise at least 60% CD56⁺CD3⁻CD11a⁺ natural killer cells. In certain aspects, the three-stage method disclosed herein produces natural killer cells that comprise at least 80% CD56⁺CD3⁻CD11a⁺ natural killer cells. In certain aspects, the three-stage method disclosed herein produces natural killer cells that comprise at least 20%, 30%, 40%, 50%, 60%, 70% or 80% CD56⁺CD3⁻CD11a⁺ natural killer cells or a percent in between a range defined by any two of the aforementioned percentages.

In certain aspects, the three-stage method produces natural killer cells that exhibit at least 20% cytotoxicity against K562 cells when said natural killer cells and said K562 cells are co-cultured in vitro at a ratio of 10:1. In certain aspects, the three-stage method produces natural killer cells that exhibit at least 35% cytotoxicity against the K562 cells when said natural killer cells and said K562 cells are co-cultured in vitro at a ratio of 10:1. In certain aspects, the three-stage method produces natural killer cells that exhibit at least 45% cytotoxicity against the K562 cells when said natural killer cells and said K562 cells are co-cultured in vitro at a ratio of 10:1. In certain aspects, the three-stage method produces natural killer cells that exhibit at least 60% cytotoxicity against the K562 cells when said natural killer cells and said K562 cells are co-cultured in vitro at a ratio of 10:1. In certain aspects, the three-stage method produces natural killer cells that exhibit at least 75% cytotoxicity against the K562 cells when said natural killer cells and said K562 cells are co-cultured in vitro at a ratio of 10:1. In certain aspects, the three-stage method produces natural killer cells that exhibit at least 35%, 45%, 55%, 65% or 75% cytotoxicity against the K562 cells when said natural killer cells and said K562 cells are co-cultured in vitro at a ratio of 10:1, or a percent in between a range defined by any two of the aforementioned percentages.

In certain aspects, after said third culturing step, said third population of cells, e.g., said population of GM NK cells, is cryopreserved. In certain aspects, after said fourth step, said fourth population of cells, e.g., said population of GM NK cells, is cryopreserved.

In certain aspects, provided herein are populations of cells comprising natural killer cells, i.e., natural killers cells produced by a three-stage method described herein. Accordingly, provided herein is an isolated natural killer cell population produced by a three-stage method described herein. In a specific alternative, said natural killer cell population comprises at least 20% CD56⁺CD3⁻ natural killer cells. In a specific alternative, said natural killer cell population comprises at least 40% CD56⁺CD3⁻ natural killer cells. In a specific alternative, said natural killer cell population comprises at least 60% CD56⁺CD3⁻ natural killer cells. In a specific alternative, said natural killer cell population comprises at least 80% CD56⁺CD3⁻ natural killer cells. In a specific alternative, said natural killer cell population comprises at least 60% CD16⁻ cells. In a specific alternative, said natural killer cell population comprises at least 80% CD16⁻ cells. In a specific alternative, said natural killer cell population comprises at least 20%, 40%, 60% or 80% CD56⁺CD3⁻ natural killer cells or a percent in between a range defined by any two of the aforementioned percentages. In a specific alternative, said natural killer cell population comprises at least 20% CD94⁺ cells. In a specific alternative, said natural killer cell population comprises at least 40% CD94⁺ cells.

In certain aspects, provided herein is a population of natural killer cells that is CD56⁺CD3⁻CD117⁺CD11a⁺, wherein said natural killer cells express perform and/or EOMES, and do not express one or more of RORγt, aryl hydrocarbon receptor (AHR), and/or IL1R1. In certain aspects, said natural killer cells express perform and EOMES, and do not express any of RORγt, aryl hydrocarbon receptor, and/or IL1R1. In certain aspects, said natural killer cells additionally express T-bet, GZMB, NKp46, NKp30, and/or NKG2D. In certain aspects, said natural killer cells express CD94. In certain aspects, said natural killer cells do not express CD94.

In certain aspects, provided herein is a method of producing a cell population comprising GM NK cells, comprising (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and/or IL-15, and lacking each of a stem cell mobilizing agent and/or LMWH, to produce a third population of cells; and (d) separating CD11a⁺ cells and CD11a⁻ cells from the third population of cells; and (e) combining the CD11a⁺ cells with the CD11a⁻ cells in a ratio of 50:1, 40:1, 30:1, 20:1, 10:1, 5:1, 4:1, 3:1, 2:1, 1:1, 1:2, 1:3, 1:4, 1:5, 1:10, 1:20, 1:30, 1:40, or 1:50 to produce a fourth population of cells or a ratio in between a range defined by any two of the aforementioned ratios. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin. In certain aspects, in the fourth population of cells, the CD11a⁺ cells and CD11a− cells are combined in a ratio of 50:1, 20:1, 10:1, 5:1, or 1:1 or any ratio in between a range defined by any two of the aforementioned ratios. In certain aspects, in the fourth population of cells, the CD11a⁺ cells and CD11a⁻ cells are combined in a ratio of 50:1. In certain aspects, in the fourth population of cells, the CD11a⁺ cells and CD11a⁻ cells are combined in a ratio of 20:1. In certain aspects, in the fourth population of cells, the CD11a⁺ cells and CD11a⁻ cells are combined in a ratio of 10:1. In certain aspects, in the fourth population of cells, the CD11a⁺ cells and CD11a⁻ cells are combined in a ratio of 5:1. In certain aspects, in the fourth population of cells, the CD11a⁺ cells and CD11a⁻ cells are combined in a ratio of 1:1. In certain aspects, in the fourth population of cells, the CD11a⁺ cells and CD11a⁻ cells are combined in a ratio of 1:5. In certain aspects, in the fourth population of cells, the CD11a⁺ cells and CD11a⁻ cells are combined in a ratio of 1:10. In certain aspects, in the fourth population of cells, the CD11a⁺ cells and CD11a⁻ cells are combined in a ratio of 1:20. In certain aspects, in the fourth population of cells, the CD11a⁺ cells and CD11a⁻ cells are combined in a ratio of 1:50.

5. Isolation of NK Cells

Methods of isolating natural killer cells are known in the art and can be used to isolate the NK cells, e.g., the GM NK cells. For example, NK cells can be isolated or enriched, for example, by staining cells, in one alternative, with antibodies to CD56 and CD3, and selecting for CD56⁺CD3⁻ cells. In certain alternatives, the NK cells are enriched for CD56⁺CD3⁻ cells in comparison with total cells produced using the three-stage method, described herein. NK cells, e.g., cells produced using the three-stage method, described herein, can be isolated using a commercially available kit, for example, the NK Cell Isolation Kit (Miltenyi Biotec). NK cells, e.g., cells produced using the three-stage method, described herein, can also be isolated or enriched by removal of cells other than NK cells in a population of cells that comprise the NK cells, e.g., cells produced using the three-stage method, described herein. For example, NK cells, e.g., cells produced using the three-stage method, described herein, may be isolated or enriched by depletion of cells displaying non-NK cell markers using, e.g., antibodies to one or more of CD3, CD4, CD14, CD19, CD20, CD36, CD66b, CD123, HLA DR and/or CD235a (glycophorin A). Negative isolation can be carried out using a commercially available kit, e.g., the NK Cell Negative Isolation Kit (Dynal Biotech). Cells isolated by these methods may be additionally sorted, e.g., to separate CD11a⁺ and CD11a⁻ cells, and/or CD117⁺ and CD117⁻ cells, and/or CD16⁺ and CD16⁻ cells, and/or CD94⁺ and CD94⁻. In certain alternatives, cells, e.g., cells produced by the three-step methods described herein, are sorted to separate CD11a⁺ and CD11a⁻ cells. In specific alternatives, CD11a⁺ cells are isolated. In certain alternatives, the cells are enriched for CD11a⁺ cells in comparison with total cells produced using the three-stage method, described herein. In specific alternatives, CD11a⁻ cells are isolated. In certain alternatives, the cells are enriched for CD11a⁻ cells in comparison with total cells produced using the three-stage method, described herein. In certain alternatives, cells are sorted to separate CD117⁺ and CD117⁻ cells. In specific alternatives, CD117⁺ cells are isolated. In certain alternatives, the cells are enriched for CD117⁺ cells in comparison with total cells produced using the three-stage method, described herein. In specific alternatives, CD117⁻ cells are isolated. In certain alternatives, the cells are enriched for CD117⁻ cells in comparison with total cells produced using the three-stage method, described herein. Methods for selecting and enriching cells are known to those of skill in the art and cells may be selected by targeting cell surface proteins, for example. In certain alternatives, cells are sorted to separate CD16⁺ and CD16⁻ cells. In specific alternatives, CD16⁺ cells are isolated. In certain alternatives, the cells are enriched for CD16⁺ cells in comparison with total cells produced using the three-stage method, described herein. In specific alternatives, CD16⁻ cells are isolated. In certain alternatives, the cells are enriched for CD16⁻ cells in comparison with total cells produced using the three-stage method, described herein. In certain alternatives, cells are sorted to separate CD94⁺ and CD94⁻ cells. In specific alternatives, CD94⁺ cells are isolated. In certain alternatives, the cells are enriched for CD94⁺ cells in comparison with total cells produced using the three-stage method, described herein. In specific alternatives, CD94⁻ cells are isolated. In certain alternatives, the cells are enriched for CD94⁻ cells in comparison with total cells produced using the three-stage method, described herein. In certain alternatives, isolation is performed using magnetic separation. In certain alternatives, isolation is performed using flow cytometry.

In one alternative, NK cells, e.g., the GM NK cells are isolated or enriched by selecting for CD56⁺CD3⁻CD94⁺CD11a⁺ cells. In certain alternatives, the NK cells are enriched for CD56⁺CD3⁻CD94⁺CD11a⁺ cells in comparison with total cells produced using the three-stage method, described herein. In one alternative, NK cells are isolated or enriched by selecting for CD56⁺CD3⁻CD94⁺CD11a⁺CD117⁻ cells. In certain alternatives, the NK cells are enriched for CD56⁺CD3⁻CD94⁺CD11a⁺CD117⁻ cells in comparison with total cells produced using the three-stage method, described herein.

Cell separation can be accomplished by, e.g., flow cytometry, fluorescence-activated cell sorting (FACS), or, in one alternative, magnetic cell sorting using microbeads conjugated with specific antibodies. The cells may be isolated, e.g., using a magnetic activated cell sorting (MACS) technique, a method for separating particles based on their ability to bind magnetic beads (e.g., 0.5-100 μm diameter) that comprise one or more specific antibodies, e.g., anti-CD56 antibodies. Magnetic cell separation can be performed and automated using, e.g., an AUTOMACS™ Separator (Miltenyi). A variety of useful modifications can be performed on the magnetic microspheres, including covalent addition of antibody that specifically recognizes a particular cell surface molecule or hapten. The beads are then mixed with the cells to allow binding. Cells are then passed through a magnetic field to separate out cells having the specific cell surface marker. In one alternative, these cells can then isolated and re-mixed with magnetic beads coupled to an antibody against additional cell surface markers. The cells are again passed through a magnetic field, isolating cells that bound both the antibodies. Such cells can then be diluted into separate dishes, such as microtiter dishes for clonal isolation.

6. GM NK Cells

GM NK cells provided herein include populations of NK cells produced by any of the methods described herein, as well as NK cells isolated from any tissue source, for example, a human tissue source.

a. 6.6.1 GM NK Cells Produced by Three-Stage Method

In another alternative, provided herein is an isolated GM NK cell population, wherein NK cells are produced according to the three-stage method described above, and wherein genetic modifications are introduced during one or more of the three stages, in order to produce a GM NK cell population.

In one alternative, provided herein is an isolated GM NK cell population, wherein an NK cell population is produced by a three-stage method described herein, wherein said NK cells population is genetically modified to produce a GM NK cell population, and wherein said NK cell population comprises 50% or more CD3⁻CD56⁺ cells. In certain alternatives, the CD3⁻CD56⁺ cells in said NK cell population comprises CD3⁻CD56⁺ cells that are additionally NKp46⁺. In certain alternatives, said CD3⁻CD56⁺ cells in said NK cell population comprises CD3⁻CD56⁺ cells that are additionally CD16⁻. In certain alternatives, said CD3⁻CD56⁺ cells in said NK cell population comprises CD3⁻CD56⁺ cells that are additionally CD16⁺. In certain alternatives, said CD3⁻CD56⁺ cells in said NK cell population comprises CD3⁻CD56⁺ cells that are additionally CD94⁻. In certain alternatives, said CD3⁻CD56⁺ cells in said NK cell population comprises CD3⁻CD56⁺ cells that are additionally CD94⁺. In certain alternatives, said CD3⁻CD56⁺ cells in said NK cell population comprises CD3⁻CD56⁺ cells that are additionally CD11a⁺. In certain alternatives, said CD3⁻CD56⁺ cells in said NK cell population comprises CD3⁻CD56⁺ cells that are additionally NKp30⁺. In certain alternatives, said CD3⁻CD56⁺ cells in said NK cell population comprises CD3⁻CD56⁺ cells that are additionally CD161⁺. In certain alternatives, said CD3⁻CD56⁺ cells in said NK cell population comprises CD3⁻CD56⁺ cells that are additionally DNAM-1⁺. In certain alternatives, said CD3⁻CD56⁺ cells in said NK cell population comprises CD3⁻CD56⁺ cells that are additionally T-bet⁺.

In one alternative, an NK cell population produced by a three-stage method described herein comprises cells, which are CD117⁺. In one alternative, an NK cell population produced by a three-stage method described herein comprises cells, wherein the cells are NKG2D⁺. In one alternative, an NK cell population produced by a three-stage method described herein comprises cells, wherein the cells are NKp44⁺. In one alternative, an NK cell population produced by a three-stage method described herein comprises cells, wherein the cells are CD244⁺. In one alternative, an NK cell population produced by a three-stage method described herein comprises cells, wherein the cells express perform. In one alternative, an NK cell population produced by a three-stage method described herein comprises cells, wherein the cells express EOMES. In one alternative, an NK cell population produced by a three-stage method described herein comprises cells, wherein the cells express granzyme B. In one alternative, an NK cell population produced by a three-stage method described herein comprises cells, wherein the cells secrete IFNγ, GM-CSF and/or TNFα.

7. Preservation of Cells

Cells, e.g., GM NK cells provided herein or produced using the methods described herein, e.g., GM NK cell populations produced using the three-stage method described herein, can be preserved, that is, placed under conditions that allow for long-term storage, or under conditions that inhibit cell death by, e.g., apoptosis or necrosis.

Suitable cryopreservation medium includes, but is not limited to, normal saline, culture medium including, e.g., growth medium, or cell freezing medium, for example commercially available cell freezing medium, e.g., C2695, C2639 or C6039 (Sigma); CryoStor® CS2, CryoStor® CS5 or CryoStor®CS10 (BioLife Solutions). In one alternative, cryopreservation medium comprises DMSO (dimethylsulfoxide), at a concentration of, e.g., 1, 2, 3, 4, 5, 6, 7, 8, 9 or 10% (v/v) or any percent v/v in between a range defined by any two of the aforementioned percentages. Cryopreservation medium may comprise additional agents, for example, methylcellulose, dextran, albumin (e.g., human serum albumin), trehalose, and/or glycerol. In certain alternatives, the cryopreservation medium comprises about 1%-10% DMSO, about 25%-75% dextran and/or about 20-60% human serum albumin (HSA). In certain alternatives, the cryopreservation medium comprises 1%, 2%, 3%, 4%, 5%, 6%, 7%, 8%, 9% or 10% DMSO or any percentage of DMSO in between a range defined by any two of the aforementioned percentages. In certain alternatives, the cryopreservation medium comprises 25%, 35%, 45%, 55%, 65%, 70%, 75% dextran, or any percentage of dextran in between a range defined by any two of the aforementioned percentages. In certain alternatives, the cryopreservation medium comprises 20%, 30%, 40%, 50% or 60% HSA, or any percentage of HSA in between a range defined by any two of the aforementioned percentages. In certain alternatives, the cryopreservation medium comprises 1%-10% DMSO, 25%-75% trehalose and/or 20-60% human HSA. In certain alternatives, the cryopreservation medium comprises 1%, 2%, 3%, 4%, 5%, 6%, 7%, 8%, 9% or 10% DMSO or any percentage of DMSO in between a range defined by any two of the aforementioned percentages. In certain alternatives, the cryopreservation medium comprises 25%, 35%, 45%, 55%, 65%, 70%, 75% trehalose, or any percentage of trehalose in between a range defined by any two of the aforementioned percentages. In certain alternatives, the cryopreservation medium comprises 20%, 30%, 40%, 50% or 60% HSA, or any percentage of HSA in between a range defined by any two of the aforementioned percentages. In a specific alternative, the cryopreservation medium comprises 5% DMSO, 55% dextran and 40% HSA. In a more specific alternative, the cryopreservation medium comprises 5% DMSO, 55% dextran (10% w/v in normal saline) and 40% HSA. In another specific alternative, the cryopreservation medium comprises 5% DMSO, 55% trehalose and 40% HSA. In a more specific alternative, the cryopreservation medium comprises 5% DMSO, 55% trehalose (10% w/v in normal saline) and 40% HSA. In another specific alternative, the cryopreservation medium comprises CryoStor® CS5. In another specific alternative, the cryopreservation medium comprises CryoStor®CS10.

Cells provided herein can be cryopreserved by any of a variety of methods, and at any stage of cell culturing, expansion or differentiation. For example, cells provided herein can be cryopreserved right after isolation from the origin tissues or organs, e.g., placental perfusate or umbilical cord blood, or during, or after either the first, second, or third step of the methods outlined above. In certain alternatives, the hematopoietic cells, e.g., hematopoietic stem or progenitor cells are cryopreserved within 1, 5, 10, 15, 20, 30, 45 minutes or within 1, 2, 4, 6, 10, 12, 18, 20 or 24 hours after isolation from the origin tissues or organs or for a time that is within a range defined by any two of the aforementioned time points. In certain alternatives, the hematopoietic cells, e.g., hematopoietic stem or progenitor cells are cryopreserved within 1, 5, 10, 15, 20, 30, 45 minutes or any number of minutes within a range defined by any two of the aforementioned number of minutes or within 1, 2, 4, 6, 10, 12, 18, 20 or 24 hours after isolation from the origin tissues or organs or within a range defined by any two of the aforementioned time points. In certain alternatives, said cells are cryopreserved within 1, 2 or 3 days after isolation from the origin tissues or organs. In certain alternatives, said cells are cryopreserved after being cultured in a first medium as described above, for 1, 2, 3, 4, 5, 6, 7, 8, 9, 10, 11, 12, 13, 14, 15, 16, 17, 18, 19, 20, 21, 22, 23, 24, 25, 26, 27 or 28 days or any number of days in between a range defined by any two of the aforementioned number of days. In some alternatives, said cells are cryopreserved after being cultured in a first medium as described above, for 1, 2, 3, 4, 5, 6, 7, 8, 9, 10, 11, 12, 13, 14, 15, 16, 17, 18, 19, 20, 21, 22, 23, 24, 25, 26, 27 or 28 days or any number of days in between a range defined by any two aforementioned number of days, and in a second medium for 1, 2, 3, 4, 5, 6, 7, 8, 9, 10, 11, 12, 13, 14, 15, 16, 17, 18, 19, 20, 21, 22, 23, 24, 25, 26, 27 or 28 days or any number of days in between a range defined by any two aforementioned number of days as described above. In some alternatives, when NK cells are made using a three-stage method described herein, said cells are cryopreserved after being cultured in a first medium 1, 2, 3, 4, 5, 6, 7, 8, 9, 10, 11, 12, 13, 14, 15, 16, 17, 18, 19, 20, 21, 22, 23, 24, or 25 days or any number of days in between a range defined by any two of the aforementioned number of days; and/or after being cultured in a second medium 1, 2, 3, 4, 5, 6, 7, 8, 9, 10, 11, 12, 13, 14, 15, 16, 17, 18, 19, 20, 21, 22, 23, 24, or 25 days or any number of days in between a range defined by any two of the aforementioned number of days; and/or after being cultured in a third medium about 1, 2, 3, 4, 5, 6, 7, 8, 9, 10, 11, 12, 13, 14, 15, 16, 17, 18, 19, 20, 21, 22, 23, 24, or 25 days or any number of days in between a range defined by any two of the aforementioned number of days. In a specific alternative, NK cells, e.g. GM NK cells, are made using a three-stage method described herein, and said cells are cryopreserved after being cultured in a first medium for 10 days; after being cultured in a second medium for 4 days; and after being cultured in a third medium for 21 days.

In one aspect, provided herein is a method of cryopreserving a population of NK cells, e.g., GM NK cells. In one alternative, said method comprises: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and/or IL-15, and lacking a stem cell mobilizing agent and/or LMWH, to produce a third population of cells, wherein the third population of cells comprises natural killer cells that are CD56⁺, CD3⁻, CD16⁻ or CD16⁺, and CD94⁺ or CD94⁻, and wherein at least 70%, or at least 80%, 85%, 90%, or 95% or a percentage within a range defined by any two of the aforementioned percentages of the natural killer cells are viable, and next, cryopreserving the NK cells in a cryopreservation medium. In certain alternatives, said first medium and/or said second medium lack leukemia inhibiting factor (LIF) and/or macrophage inflammatory protein-1 alpha (MIP-1a). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin. In a specific alternative, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain alternatives, the method includes no intermediary steps.

In one alternative, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and IL-15, and lacking LMWH, to produce a third population of cells; wherein the third population of cells comprises natural killer cells that are CD56⁺, CD3⁻, and CD11a⁺ and next, cryopreserving the NK cells in a cryopreservation medium. Cell mobilizing agents are known to those skilled in the art and may include CXCR4 antagonists such as Plerixafor, for example. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin. In a specific alternative, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain alternatives, the method includes no intermediary steps.

In one alternative, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and/or IL-15, and lacking each of stem cell factor (SCF) and/or LMWH, to produce a third population of cells; wherein the third population of cells comprises natural killer cells that are CD56⁺, CD3⁻, and CD11a⁺ and next, cryopreserving the NK cells in a cryopreservation medium. In certain alternatives, said first medium and/or said second medium lack leukemia inhibiting factor (LIF) and/or macrophage inflammatory protein-1 alpha (MIP-1a). In certain alternatives, said third medium lacks LIF, MIP-1α, and/or FMS-like tyrosine kinase-3 ligand (Flt-3L). In specific alternatives, said first medium and said second medium lack LIF and MIP-1α, and said third medium lacks LIF, MIP-1α, and/or Flt3L. In certain alternatives, none of the first medium, second medium or third medium comprises heparin, e.g., low-molecular weight heparin. In a specific alternative, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain alternatives, the method includes no intermediary steps.

In one alternative, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; and (c) culturing the second population of cells in a third medium comprising IL-2 and/or IL-15, and lacking each of SCF, a stem cell mobilizing agent, and/or LMWH, to produce a third population of cells; wherein the third population of cells comprises natural killer cells that are CD56⁺, CD3⁻, and CD11a⁺ and next, cryopreserving the NK cells in a cryopreservation medium. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin. In a specific alternative, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain alternatives, the method includes no intermediary steps.

In one alternative, said method comprises: (a) culturing hematopoietic stem or progenitor cells in a first medium comprising a stem cell mobilizing agent and thrombopoietin (Tpo) to produce a first population of cells; (b) culturing the first population of cells in a second medium comprising a stem cell mobilizing agent and interleukin-15 (IL-15), and lacking Tpo, to produce a second population of cells; (c) culturing the second population of cells in a third medium comprising IL-2 and/or IL-15, and lacking each of a stem cell mobilizing agent and/or LMWH, to produce a third population of cells; and (d) isolating CD11a⁺ cells from the third population of cells to produce a fourth population of cells; wherein the fourth population of cells comprises natural killer cells that are CD56⁺, CD3⁻, and CD11a⁺ and next, cryopreserving the NK cells in a cryopreservation medium. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin. In a specific alternative, said cryopreservation step further comprises (1) preparing a cell suspension solution; (2) adding cryopreservation medium to the cell suspension solution from step (1) to obtain cryopreserved cell suspension; (3) cooling the cryopreserved cell suspension from step (3) to obtain a cryopreserved sample; and (4) storing the cryopreserved sample below −80° C. In certain alternatives, the method includes no intermediary steps.

Cells provided herein can be cooled in a controlled-rate freezer, e.g., at 0.1, 0.3, 0.5, 1, or 2° C./min or any temperature in between a range defined by any two of the aforementioned temperatures during cryopreservation. In one alternative, the cryopreservation temperature is −80° C. to −180° C., or −125° C. to −140° C. Cryopreserved cells can be transferred to liquid nitrogen prior to thawing for use. In some alternatives, for example, once the ampoules have reached −90° C., they are transferred to a liquid nitrogen storage area. Cryopreserved cells can be thawed at a temperature of 25° C. to 40° C., more specifically can be thawed to a temperature of 37° C. Cryopreserved cells can be thawed at a temperature of 25° C., 35° C., 40° C. or 40° C., or any temperature in between a range defined by any two aforementioned temperatures. In certain alternatives, the cryopreserved cells are thawed after being cryopreserved for 1, 2, 4, 6, 10, 12, 18, 20 or 24 hours or any number of hours in between a range defined by any two of the aforementioned values, or for 1, 2, 3, 4, 5, 6, 7, 8, 9, 10, 11, 12, 13, 14, 15, 16, 17, 18, 19, 20, 21, 22, 23, 24, 25, 26, 27 or 28 days or any number of days in between a range defined by any two of the aforementioned values. In certain alternatives, the cryopreserved cells are thawed after being cryopreserved for 1, 2, 3, 4, 5, 6, 7, 8, 9, 10, 11, 12, 13, 14, 15, 16, 17, 18, 19, 20, 21, 22, 23, 24, 25, 26, 27 or 28 months or any number of months in between a range defined by any two of the aforementioned values. In certain alternatives, the cryopreserved cells are thawed after being cryopreserved for 1, 2, 3, 4, 5, 6, 7, 8, 9 or 10 years or any number of years in between a range defined by any two of the aforementioned values.

Suitable thawing medium includes, but is not limited to, normal saline, plasmalyte culture medium including, for example, growth medium, e.g., RPMI medium. In certain alternatives, the thawing medium comprises one or more of medium supplements (e.g., nutrients, cytokines and/or factors). Medium supplements suitable for thawing cells provided herein include, for example without limitation, serum such as human serum AB, fetal bovine serum (FBS) or fetal calf serum (FCS), vitamins, human serum albumin (has), bovine serum albumin (BSA), amino acids (e.g., L-glutamine), fatty acids (e.g., oleic acid, linoleic acid or palmitic acid), insulin (e.g., recombinant human insulin), transferrin (iron saturated human transferrin), β-mercaptoethanol, stem cell factor (SCF), Fms-like-tyrosine kinase 3 ligand (Flt3-L), cytokines such as interleukin-2 (IL-2), interleukin-7 (IL-7), interleukin-15 (IL-15), thrombopoietin (Tpo) or heparin. In a specific alternative, the thawing medium useful in the methods provided herein comprises RPMI. In another specific alternative, said thawing medium comprises plasmalyte. In another specific alternative, said thawing medium comprises 0.5-20% FBS. In another specific alternative, said thawing medium comprises 0.5, 1, 5, 15, 15 or 20% FBS or any percentage of FBS in between a ranged defined by any two of the aforementioned percentages. In another specific alternative, said thawing medium comprises 1, 2, 5, 10, 15 or 20% FBS. In another specific alternative, said thawing medium comprises 0.5%-20% HSA. In another specific alternative, said thawing medium comprises 0.5, 1, 5, 15, 15 or 20% HSA or any percentage of HSA in between a ranged defined by any two of the aforementioned percentages. In another specific alternative, said thawing medium comprises 1, 2.5, 5, 10, 15, or 20% HSA. In a more specific alternative, said thawing medium comprises RPMI and 10% FBS. In another more specific alternative, said thawing medium comprises plasmalyte and 5% HSA.

The cryopreservation methods provided herein can be optimized to allow for long-term storage, or under conditions that inhibit cell death by, e.g., apoptosis or necrosis. In one alternatives, the post-thaw cells comprise greater than 60%, 65%, 70%, 75%, 80%, 85%, 90%, 95% or 98% of viable cells, as determined by, e.g., automatic cell counter or trypan blue method. In one alternatives, the post-thaw cells comprise greater than 60%, 65%, 70%, 75%, 80%, 85%, 90%, 95% or 98% of viable cells or any percentage in between a range defined by any two of the aforementioned percentages. In another alternative, the post-thaw cells comprise 0.5, 1, 5, 10, 15, 20 or 25% of dead cells. In another alternative, the post-thaw cells comprise 0.5, 1, 5, 10, 15, 20 or 25% of dead cells or any percentage of dead cells in between a range defined by any two of the aforementioned percentages. In another alternative, the post-thaw cells comprise 0.5, 1, 5, 10, 15, 20 or 25% of early apoptotic cells. In another alternative, the post-thaw cells comprise 0.5, 1, 5, 10, 15, 20 or 25% of early apoptotic cells or any percentage of early apoptotic cells in between a range defined by any two of the aforementioned percentages. In another alternative, 0.5, 1, 5, 10, 15 or 20% of post-thaw cells undergo apoptosis after 1, 2, 3, 4, 5, 6, 7, 8, 9, 10, 11, 12, 13, 14, 15, 16, 17, 18, 19, 20, 21, 22, 23, 24, 25, 26, 27 or 28 days after being thawed, e.g., as determined by an apoptosis assay (e.g., TO-PRO3 or AnnV/PI Apoptosis assay kit). In certain alternatives, the post-thaw cells are re-cryopreserved after being cultured, expanded or differentiated using methods provided herein.

8. Compositions Comprising GM NK Cells

Compositions, such as pharmaceutical compositions, comprising GM NK cells provided herein include compositions comprising populations of NK cells produced by any of the methods described herein, as well as compositions comprising NK cells isolated from any tissue source, for example, a human tissue source.

a. 6.8.1 GM NK Cells Produced Using the Three-Stage Method

In some alternatives, provided herein is a composition, e.g., a pharmaceutical composition, comprising an isolated NK cell population, e.g., a GM NK cell population. In a specific alternative, said isolated NK cell population is produced from hematopoietic cells, e.g., hematopoietic stem or progenitor cells isolated from placental perfusate, umbilical cord blood, and/or peripheral blood. In another specific alternative, said isolated NK cell population comprises at least 50% of cells in the composition. In another specific alternative, said isolated NK cell population, e.g., CD3⁻CD56⁺ cells, comprises at least 80%, 85%, 90%, 95%, 98% or 99% of cells in the composition. In certain alternatives, no more than 5%, 10%, 15%, 20%, 25%, 30%, 35%, or 40% of the cells in said isolated NK cell population are CD3⁻CD56⁺ cells. In certain alternatives, said CD3⁻CD56⁺ cells are CD16⁻.

NK cell populations, e.g., GM NK cell populations, can be formulated into pharmaceutical compositions for use in vivo. Such pharmaceutical compositions comprise a population of NK cells in a pharmaceutically-acceptable carrier, e.g., a saline solution or other accepted physiologically-acceptable solution for in vivo administration. Pharmaceutical compositions of the invention can comprise any of the NK cell populations described elsewhere herein.

The pharmaceutical compositions described herein comprise populations of NK cells that comprise 50% viable cells or more (that is, e.g., at least 50% of the cells in the population are functional or living). Preferably, at least 60% of the cells in the population are viable. More preferably, at least 70%, 80%, 90%, 95%, or 99% of the cells in the population in the pharmaceutical composition are viable or any percentage within a range defined by any two of the aforementioned percentages.

The pharmaceutical compositions described herein can comprise one or more compounds that, e.g., facilitate engraftment; stabilizers such as albumin, dextran 40, gelatin, and/or hydroxyethyl starch.

When formulated as an injectable solution, in one alternative, the pharmaceutical composition can comprise 1.25% HSA and 2.5% dextran. Other injectable formulations, suitable for the administration of cellular products, may be used.

In one alternative, the compositions, e.g., pharmaceutical compositions, provided herein are suitable for systemic or local administration. In specific alternatives, the compositions, e.g., pharmaceutical compositions, provided herein are suitable for parenteral administration. In specific alternatives, the compositions, e.g., pharmaceutical compositions, provided herein are suitable for injection, infusion, intravenous (IV) administration, intrafemoral administration, or intratumor administration. In specific alternatives, the compositions, e.g., pharmaceutical compositions, provided herein are suitable for administration via a device, a matrix, or a scaffold. In specific alternatives, the compositions, e.g., pharmaceutical compositions provided herein are suitable for injection. In specific alternatives, the compositions, e.g., pharmaceutical compositions, provided herein are suitable for administration via a catheter. In specific alternatives, the compositions, e.g., pharmaceutical compositions, provided herein are suitable for local injection. In more specific alternatives, the compositions, e.g., pharmaceutical compositions, provided herein are suitable for local injection directly into a solid tumor (e.g., a sarcoma). In specific alternatives, the compositions, e.g., pharmaceutical compositions, provided herein are suitable for injection by syringe. In specific alternatives, the compositions, e.g., pharmaceutical compositions, provided herein are suitable for administration via guided delivery. In specific alternatives, the compositions, e.g., pharmaceutical compositions, provided herein are suitable for injection aided by laparoscopy, endoscopy, ultrasound, computed tomography, magnetic resonance, or radiology.

In certain alternatives, the compositions, e.g., pharmaceutical compositions provided herein, comprising NK cells, e.g., GM NK cells, are provided as pharmaceutical grade administrable units. Such units can be provided in discrete volumes, e.g., 15 mL, 20 mL, 25 mL, 30 ml, 35 mL, 40 mL, 45 mL, 50 mL, 55 mL, 60 mL, 65 mL, 70 mL, 75 mL, 80 mL, 85 mL, 90 mL, 95 mL, 100 mL, 150 mL, 200 mL, 250 mL, 300 mL, 350 mL, 400 mL, 450 mL, 500 mL, or the like or any volume in between a range defined by any two of the aforementioned volume amounts. Such units can be provided so as to contain a specified number of cells, e.g., GM NK cells, e.g., 1×10⁴, 5×10⁴, 1×10⁵, 5×10⁵, 1×10⁶, 5×10⁶, 1×10⁷, 5×10⁷, 1×10⁸, 5×10⁸ or more cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit or any number of cells per unit in between a range defined by any two of the aforementioned values. In specific alternatives, the units can comprise about, at least about, or at most about 1×10⁴, 5×10⁴, 1×10⁵, 5×10⁵, 1×10⁶, 5×10⁶ or more GM NK cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more cells per unit or any number of cells milliter or per unit within a range defined by any two aforementioned values. Such units can be provided to contain specified numbers of NK cells or NK cell populations and/or any of the other cells. In specific alternatives, the NK cells are present in ratios as provided herein.

In another specific alternative, said isolated NK cells, e.g., GM NK cells, in said composition are from a single individual. In a more specific alternative, said isolated NK cells comprise NK cells from at least two different individuals. In another specific alternative, said isolated NK cells in said composition are from a different individual than the individual for whom treatment with the NK cells is intended. In another specific alternative, said NK cells have been contacted or brought into proximity with an immunomodulatory compound or thalidomide in an amount and for a time sufficient for said NK cells to express detectably more granzyme B and/or perform than an equivalent number of natural killer cells, i.e. NK cells not contacted or brought into proximity with said immunomodulatory compound or thalidomide. In another specific alternative, said composition additionally comprises or is provided in a product combination or in conjunction (e.g., before, during, or after but separately) with an immunomodulatory compound or thalidomide. In certain alternatives, the immunomodulatory compound is a compound described below. See, e.g., U.S. Pat. No. 7,498,171, the disclosure of which is hereby incorporated by reference in its entirety. In certain alternatives, the immunomodulatory compound is an amino-substituted isoindoline. In one alternative, the immunomodulatory compound is 3-(4-amino-1-oxo-1,3-dihydroisoindol-2-yl)-piperidine-2,6-dione; 3-(4′aminoisolindoline-1′-one)-1-piperidine-2,6-dione; 4-(amino)-2-(2,6-dioxo(3-piperidyl))-isoindoline-1,3-dione; or 4-Amino-2-(2,6-dioxopiperidin-3-yl)isoindole-1,3-dione. In another alternative, the immunomodulatory compound is pomalidomide, or lenalidomide. In another alternative, said immunomodulatory compound is a compound having the structure:

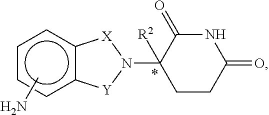

wherein one of X and Y is C═O, the other of X and Y is C═O or CH₂, and R² is hydrogen or lower alkyl, or a pharmaceutically acceptable salt, hydrate, solvate, clathrate, enantiomer, diastereomer, racemate, or mixture of stereoisomers thereof. In another alternative, said immunomodulatory compound is a compound having the structure:

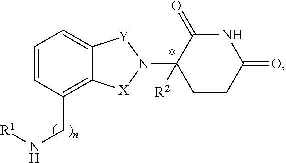

wherein one of X and Y is C═O and the other is CH₂ or C═O;
R¹ is H, (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, C(O)R³, C(S)R³, C(O)OR⁴, (C₁-C₈)alkyl-N(R⁶)₂, (C₁-C₈)alkyl-OR⁵, (C₁-C₈)alkyl-C(O)OR⁵, C(O)NHR³, C(S)NHR³, C(O)NR³R^{3′}, C(S)NR³R^{3′} or (C₁-C₈)alkyl-O(CO)R⁵;
R² is H, F, benzyl, (C₁-C₈)alkyl, (C₂-C₈)alkenyl, or (C₂-C₈)alkynyl;
R³ and R^{3′} are independently (C₁-C₈)alkyl, (C₃-C₇)cycloalkyl, (C₂-C₈)alkenyl, (C₂-C₈)alkynyl, benzyl, aryl, (C₀-C₄)alkyl-(C₁-C₆)heterocycloalkyl, (C₀-C₄)alkyl-(C₂-C₅)heteroaryl, (C₀-C₈)alkyl-N(R⁶)₂, (C₁-C₈)alkyl-OR⁵, (C₁-C₈)alkyl-C(O)OR⁵, (C₁-C₈)alkyl-O(CO)R⁵, or C(O)OR⁵;
R⁴ is (C₁-C₈)alkyl, (C₂-C₈)alkenyl, (C₂-C₈)alkynyl, (C₁-C₄)alkyl-OR⁵, benzyl, aryl, (C₀-C₄)alkyl-(C₁-C₆)heterocycloalkyl, or (C₀-C₄)alkyl-(C₂-C₅)heteroaryl;
R⁵ is (C₁-C₈)alkyl, (C₂-C₈)alkenyl, (C₂-C₈)alkynyl, benzyl, aryl, or (C₂-C₅)heteroaryl;
each occurrence of R⁶ is independently H, (C₁-C₈)alkyl, (C₂-C₈)alkenyl, (C₂-C₈)alkynyl, benzyl, aryl, (C₂-C₅)heteroaryl, or (C₀-C₈)alkyl-C(O)O—R⁵ or the R⁶ groups can join to form a heterocycloalkyl group;
n is 0 or 1; and
represents a chiral-carbon center;
or a pharmaceutically acceptable salt, hydrate, solvate, clathrate, enantiomer, diastereomer, racemate, or mixture of stereoisomers thereof. In another alternative, said immunomodulatory compound is a compound having the structure

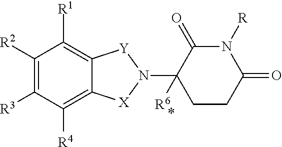

wherein:
one of X and Y is C═O and the other is CH₂ or C═O;
R is H or CH₂OCOR′;
(i) each of R¹, R², R³, or R⁴, independently of the others, is halo, alkyl of 1 to 4 carbon atoms, or alkoxy of 1 to 4 carbon atoms or (ii) one of R¹, R², R³, or R⁴ is nitro or —NHR⁵ and the remaining of R¹, R², R³, or R⁴ are hydrogen;
R⁵ is hydrogen or alkyl of 1 to 8 carbons
R⁶ hydrogen, alkyl of 1 to 8 carbon atoms, benzo, chloro, or fluoro;
R′ is R⁷—CHR¹⁰—N(R⁸R⁹);
R⁷ is m-phenylene or p-phenylene or —(CₙH₂ₙ)— in which n has a value of 0 to 4;
each of R⁸ and R⁹ taken independently of the other is hydrogen or alkyl of 1 to 8 carbon atoms, or R⁸ and R⁹ taken together are tetramethylene, pentamethylene, hexamethylene, or —CH₂CH₂X₁CH₂CH₂— in which X₁ is —O—, —S—, or —NH—;
R¹⁰ is hydrogen, alkyl of to 8 carbon atoms, or phenyl; and
represents a chiral-carbon center;

or a pharmaceutically acceptable salt, hydrate, solvate, clathrate, enantiomer, diastereomer, racemate, or mixture of stereoisomers thereof.

In another specific alternative, the compositions described herein additionally comprises or is administered in a product combination or in conjunction with one or more anticancer compounds, e.g., one or more of the anticancer compounds described below.

In a more specific alternative, the composition comprises NK cells from another source, or made by another method, whether genetically modified or not. In a specific alternative, said other source is placental blood and/or umbilical cord blood. In another specific alternative, said other source is peripheral blood. In more specific alternatives, the NK cell population in said composition is combined with NK cells from another source, or made by another method in a ratio of about 100:1, 95:5, 90:10, 85:15, 80:20, 75:25, 70:30, 65:35, 60:40, 55:45: 50:50, 45:55, 40:60, 35:65, 30:70, 25:75, 20:80, 15:85, 10:90, 5:95, 100:1, 95:1, 90:1, 85:1, 80:1, 75:1, 70:1, 65:1, 60:1, 55:1, 50:1, 45:1, 40:1, 35:1, 30:1, 25:1, 20:1, 15:1, 10:1, 5:1, 1:1, 1:5, 1:10, 1:15, 1:20, 1:25, 1:30, 1:35, 1:40, 1:45, 1:50, 1:55, 1:60, 1:65, 1:70, 1:75, 1:80, 1:85, 1:90, 1:95, 1:100, or the like or any ratio in between a range defined by any two aforementioned ratios.

In another specific alternative, the composition comprises an NK cell population produced using the three-stage method described herein and either isolated placental perfusate or isolated placental perfusate cells. In a more specific alternative, said placental perfusate is from the same individual as said NK cell population. In another more specific alternative, said placental perfusate comprises placental perfusate from a different individual than said NK cell population. In another specific alternative, all, or substantially all (e.g., greater than 90%, 95%, 98% or 99%) of cells in said placental perfusate are fetal cells. In another specific alternative, the placental perfusate or placental perfusate cells, comprise fetal and maternal cells. In a more specific alternative, the fetal cells in said placental perfusate comprise less than 90%, 80%, 70%, 60% or 50% (but not zero) of the cells or any percentage of cells in between a range defined by any two of the aforementioned percentage in said perfusate. In another specific alternative, said perfusate is obtained by passage of a 0.9% NaCl solution through the placental vasculature. In another specific alternative, said perfusate comprises a culture medium. In another specific alternative, said perfusate has been treated to remove erythrocytes. In another specific alternative, said composition comprises an immunomodulatory compound, e.g., an immunomodulatory compound described below, e.g., an amino-substituted isoindoline compound. In another specific alternative, the composition additionally comprises one or more anticancer compounds, e.g., one or more of the anticancer compounds described below.

In another specific alternative, the composition comprises an NK cell population and placental perfusate cells. In a more specific alternative, said placental perfusate cells are from the same individual as said NK cell population. In another more specific alternative, said placental perfusate cells are from a different individual than said NK cell population. In another specific alternative, the composition comprises isolated placental perfusate and isolated placental perfusate cells, wherein said isolated perfusate and said isolated placental perfusate cells are from different individuals. In another more specific alternative of any of the above alternatives comprising placental perfusate, said placental perfusate comprises placental perfusate from at least two individuals. In another more specific alternative of any of the above alternatives comprising placental perfusate cells, said isolated placental perfusate cells are from at least two individuals. In another specific alternative, said composition comprises an immunomodulatory compound. In another specific alternative, the composition additionally comprises one or more anticancer compounds, e.g., one or more of the anticancer compounds described below.

9. Uses of GM NK Cells

The GM NK cells described herein, for example, GM NK cells produced by the three-stage method described herein, can be used in methods of providing a therapy to individuals having cancer, e.g., individuals having solid tumor cells and/or blood cancer cells, or persons having a viral infection. In some such alternatives, an effective dosage of NK cells ranges from 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, or more cells/kilogram body weight. In some such alternatives, an effective dosage of NK cells ranges from 1×10⁴ to 5×10⁴, 5×10⁴ to 1×10⁵, 1×10⁵ to 5×10⁵, 5×10⁵ to 1×10⁶, 1×10⁶ to 5×10⁶, 5×10⁶ to 1×10⁷, or more cells/kilogram body weight or any number of cells per kilogram of body weight in between a range defined by any two aforementioned values. The NK cells, e.g., GM NK cells described herein, can also be used in methods of suppressing proliferation of tumor cells.

a. 6.9.1 Treatment of Individuals Having Cancer

In one alternative, provided herein is a method of providing a therapy to an individual having a cancer, for example, a blood cancer or a solid tumor, comprises administering to said individual, preferably one that has been selected or identified to receive an anticancer therapy, a therapeutically effective amount of GM NK cells described herein, e.g., GM NK cell populations described herein. In certain alternatives, the individual has a deficiency of natural killer cells, e.g., a deficiency of NK cells active against the individual's cancer and said individual has been identified or selected as such prior to receiving the therapy. In a specific alternative, the method additionally comprises administering to said individual isolated placental perfusate or isolated placental perfusate cells, e.g., a therapeutically effective amount of placental perfusate or isolated placental perfusate cells. In some alternatives, the individual has been selected to receive the isolated placental perfusate or isolated placental perfusate cells. In another specific alternative, the method comprises additionally administering to said individual an effective amount of an immunomodulatory compound, e.g., an immunomodulatory compound described above, or thalidomide. In some alternatives, the individual has been selected to receive an immunomodulatory compound. As used herein, an “effective amount” is an amount that, e.g., results in a detectable improvement of, lessening of the progression of, or elimination of, one or more symptoms of a cancer from which the individual suffers.

Administration of an isolated population of GM NK cells or a pharmaceutical composition thereof may be systemic or local. In specific alternatives, administration is parenteral. In specific alternatives, administration of an isolated population of GM NK cells or a pharmaceutical composition thereof to a subject is by injection, infusion, intravenous (IV) administration, intrafemoral administration, or intratumor administration. In specific alternatives, administration of an isolated population of GM NK cells or a pharmaceutical composition thereof to a subject is performed with a device, a matrix, or a scaffold. In specific alternatives, administration an isolated population of GM NK cells or a pharmaceutical composition thereof to a subject is by injection. In specific alternatives, administration an isolated population of GM NK cells or a pharmaceutical composition thereof to a subject is via a catheter. In specific alternatives, the injection of GM NK cells is a local injection. In more specific alternatives, the local injection is directly into a solid tumor (e.g., a sarcoma). In specific alternatives, administration of an isolated population of GM NK cells or a pharmaceutical composition thereof to a subject is by injection by syringe. In specific alternatives, administration of an isolated population of GM NK cells or a pharmaceutical composition thereof to a subject is via guided delivery. In specific alternatives, administration of an isolated population of GM NK cells or a pharmaceutical composition thereof to a subject by injection is aided by laparoscopy, endoscopy, ultrasound, computed tomography, magnetic resonance, or radiology.

In a specific alternative, the cancer is a blood cancer, e.g., a leukemia or a lymphoma. In more specific alternatives, the cancer is an acute leukemia, e.g., acute T cell leukemia, acute myelogenous leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), or acute biphenotypic leukemia; a chronic leukemia, e.g., chronic myeloid lymphoma, chronic myelogenous leukemia (CML), chronic monocytic leukemia, chronic lymphocytic leukemia (CLL)/Small lymphocytic lymphoma, or B-cell prolymphocytic leukemia; hairy cell lymphoma; T-cell prolymphocytic leukemia; or a lymphoma, e.g., histiocytic lymphoma, lymphoplasmacytic lymphoma (e.g., Waldenström macroglobulinemia), splenic marginal zone lymphoma, plasma cell neoplasm (e.g., plasma cell myeloma, plasmacytoma, a monoclonal immunoglobulin deposition disease, or a heavy chain disease), extranodal marginal zone B cell lymphoma (MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides (Sezary syndrome), a primary cutaneous CD30-positive T cell lymphoproliferative disorder (e.g., primary cutaneous anaplastic large cell lymphoma or lymphomatoid papulosis), angioimmunoblastic T cell lymphoma, peripheral T cell lymphoma, unspecified, anaplastic large cell lymphoma, a Hodgkin's lymphoma or a nodular lymphocyte-predominant Hodgkin's lymphoma. In another specific alternative, the cancer is multiple myeloma or myelodysplastic syndrome.

In certain other specific alternatives, the cancer is a solid tumor, e.g., a carcinoma, such as an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma (e.g., a malignant melanoma), a non-melanoma skin carcinoma, or an unspecified carcinoma; a desmoid tumor; a desmoplastic small round cell tumor; an endocrine tumor; an Ewing sarcoma; a germ cell tumor (e.g., testicular cancer, ovarian cancer, choriocarcinoma, endodermal sinus tumor, germinoma, etc.); a hepatosblastoma; a hepatocellular carcinoma; a neuroblastoma; a non-rhabdomyosarcoma soft tissue sarcoma; an osteosarcoma; a retinoblastoma; a rhabdomyosarcoma; or a Wilms tumor. In another alternative, the solid tumor is pancreatic cancer or a breast cancer. In other alternatives, the solid tumor is an acoustic neuroma; an astrocytoma (e.g., a grade I pilocytic astrocytoma, a grade II low-grade astrocytoma; a grade III anaplastic astrocytoma; or a grade IV glioblastoma multiforme); a chordoma; a craniopharyngioma; a glioma (e.g., a brain stem glioma; an ependymoma; a mixed glioma; an optic nerve glioma; or a subependymoma); a glioblastoma; a medulloblastoma; a meningioma; a metastatic brain tumor; an oligodendroglioma; a pineoblastoma; a pituitary tumor; a primitive neuroectodermal tumor; or a schwannoma. In another alternative, the cancer is a prostate cancer. In another alternative, the cancer is a liver cancer. In another alternative, the cancer is a lung cancer. In another alternative, the cancer is a renal cancer.

In certain alternatives, the individual having a cancer, for example, a blood cancer or a solid tumor, e.g., an individual having a deficiency of natural killer cells, is an individual that has received a bone marrow transplant before said administering. In certain alternatives, the bone marrow transplant was in treatment of said cancer. In certain other alternatives, the bone marrow transplant was in treatment of a condition other than said cancer. In certain alternatives, the individual received an immunosuppressant in addition to said bone marrow transplant. In certain alternatives, the individual who has had a bone marrow transplant exhibits one or more symptoms of graft-versus-host disease (GVHD) at the time of said administration. In certain other alternatives, the individual who has had a bone marrow transplant is administered said cells before a symptom of GVHD has manifested.

In certain specific alternatives, the individual having a cancer, for example, a blood cancer, has received at least one dose of a TNFα inhibitor, e.g., ETANERCEPT® (Enbrel), prior to said administering. In specific alternatives, said individual received said dose of a TNFα inhibitor within 1, 2, 3, 4, 5, 6, 7, 8, 9, 10, 11 or 12 months of diagnosis of said cancer or within a range defined by any two of the aforementioned time periods. In a specific alternative, the individual who has received a dose of a TNFα inhibitor exhibits acute myeloid leukemia. In a more specific alternative, the individual who has received a dose of a TNFα inhibitor and exhibits acute myeloid leukemia further exhibits deletion of the long arm of chromosome 5 in blood cells. In another alternative, the individual having a cancer, for example, a blood cancer, exhibits a Philadelphia chromosome.

In certain other alternatives, the cancer, for example, a blood cancer or a solid tumor, in said individual is refractory to one or more anticancer drugs. In a specific alternative, the cancer is refractory to GLEEVEC® (imatinib mesylate).

In certain alternatives, the cancer, for example, a blood cancer, in said individual responds to at least one anticancer drug; in this alternative, placental perfusate, isolated placental perfusate cells, isolated natural killer cells, e.g., placental natural killer cells, e.g., placenta-derived intermediate natural killer cells, isolated combined natural killer cells, or NK cells described herein, and/or combinations thereof, and optionally an immunomodulatory compound, are added as adjunct therapy or as a combination therapy with said anticancer drug. In certain other alternatives, the individual having a cancer, for example, a blood cancer, has received at least one anticancer drug, and has relapsed, prior to said administering. In certain alternatives, the individual to receive therapy has a refractory cancer. In one alternative, the cancer treatment method with the cells described herein protects against (e.g., prevents or delays) relapse of cancer. In one alternative, the cancer treatment method described herein results in remission of the cancer for 1 month or more, 2, 3, 4, 5, 6, 7, 8, 9, 10, 11, or 12 months or more, 1 year or more, 2 years or more, 3 years or more, or 4 years or more.

In one alternative, provided herein is a method of providing a therapy to an individual having multiple myeloma, comprising administering to the individual (1) lenalidomide; (2) melphalan; and (3) GM NK cells, wherein said GM NK cells are effective to treat multiple myeloma in said individual. In a specific alternative, said GM NK cells are derived from cord blood NK cells, or NK cells produced from cord blood hematopoietic cells, e.g., hematopoietic stem cells. In another alternative, said GM NK cells have been produced by a three-stage method described herein for producing NK cells. In another alternative, said lenalidomide, melphalan, and/or GM NK cells are administered separately from each other. In certain specific alternatives of the method of treating an individual with multiple myeloma, said GM NK cells are produced by a method comprising producing NK cells by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and/or IL-15, and lacking a stem cell mobilizing agent and/or LMWH, to produce a third population of cells, wherein the third population of cells comprises natural killer cells that are CD56^{±}, CD3⁻, CD16− or CD16⁺, and CD94⁺ or CD94⁻, and wherein at least 70%, or at least 80%, 85%, 90%, or 95% of the natural killer cells are viable. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In another alternative, provided herein is a method of treating an individual having acute myelogenous leukemia (AML), comprising administering to the individual NK cells (optionally activated by pretreatment with IL2 alone, or IL-15 alone, IL2 and IL12 and IL18, IL12 and IL15, IL12 and IL18, IL2 and IL12 and IL15 and IL18, or IL2 and IL15 and IL18), wherein said NK cells are effective to treat AML in said individual. In a specific alternative, the isolated NK cell population produced using the three-stage methods described herein has been pretreated with one or more of IL2, IL12, IL18, or IL15 prior to said administering. In a specific alternative, said GM NK cells are derived from cord blood NK cells, or NK cells produced from cord blood hematopoietic cells, e.g., hematopoietic stem cells. In another alternative, said GM NK cells have been produced by a three-stage method described herein for producing NK cells. In certain specific alternatives of the method of treating an individual with AML, said NK cells are produced by a three-stage method, as described herein. In a particular alternative, the AML to be treated by the foregoing methods comprises refractory AML, poor-prognosis AML, or childhood AML. Methods known in the art for administering NK cells for the treatment of refractory AML, poor-prognosis AML, or childhood AML may be adapted for this purpose; see, e.g., Miller et al., 2005, Blood 105:3051-3057; Rubnitz et al., 2010, J Clin Oncol. 28:955-959, each of which is incorporated herein by reference in its entirety. In certain alternatives, said individual has AML that has failed at least one non-natural killer cell therapeutic against AML. In specific alternatives, said individual is 65 years old or greater, and is in first remission. In specific alternatives, said individual has been conditioned with fludarabine, cytarabine, or both prior to administering said natural killer cells.

In other specific alternatives of the method of treating an individual with AML, said GM NK cells are produced by a method comprising producing NK cells by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and/or IL-15, and lacking a stem cell mobilizing agent and/or LMWH, to produce a third population of cells, wherein the third population of cells comprises natural killer cells that are CD56⁺, CD3⁻, CD16− or CD16⁺, and CD94⁺ or CD94⁻, and wherein at least 70%, or at least 80%, 85%, 90%, or 95% of the natural killer cells are viable. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

In another alternative, provided herein is a method of treating an individual having chronic lymphocytic leukemia (CLL), comprising administering to the individual a therapeutically effective dose of (1) lenalidomide; (2) melphalan; (3) fludarabine; and (4) NK cells, e.g., GM NK cells described herein, wherein said GM NK cells are effective to treat or ameliorate or inhibit said CLL in said individual. In a specific alternative, said GM NK cells are derived from cord blood NK cells, or NK cells produced from cord blood hematopoietic stem cells. In another alternative, said GM NK cells have been produced by a three-stage method described herein for producing NK cells. In a specific alternative of any of the above methods, said lenalidomide, melphalan, fludarabine, and GM NK cells are administered to said individual separately. In certain specific alternatives of the method of providing a therapy to an individual with CLL, said GM NK cells are produced by a method comprising producing NK cells by a method comprising: culturing hematopoietic stem cells or progenitor cells, e.g., CD34⁺ stem cells or progenitor cells, in a first medium comprising a stem cell mobilizing agent and thrombopoietin (Tpo) to produce a first population of cells, subsequently culturing said first population of cells in a second medium comprising a stem cell mobilizing agent and interleukin-15 (IL-15), and lacking Tpo, to produce a second population of cells, and subsequently culturing said second population of cells in a third medium comprising IL-2 and/or IL-15, and lacking a stem cell mobilizing agent and LMWH, to produce a third population of cells, wherein the third population of cells comprises natural killer cells that are CD56⁺, CD3⁻, CD16− or CD16⁺, and CD94⁺ or CD94⁻, and wherein at least 70%, or at least 80%, 85%, 90%, or 95% of the natural killer cells are viable. In certain alternatives, said first medium and/or said second medium lack leukemia inhibiting factor (LIF) and/or macrophage inflammatory protein-1 alpha (MIP-1α). In certain alternatives, said third medium lacks LIF, MIP-1α, and/or FMS-like tyrosine kinase-3 ligand (Flt-3L). In specific alternatives, said first medium and said second medium lack LIF and/or MIP-1α, and said third medium lacks LIF, MIP-1α, and/or Flt3L. In certain alternatives, none of the first medium, second medium or third medium comprises heparin, e.g., low-molecular weight heparin.

b. 6.9.2 Suppression of Tumor Cell Proliferation

Further provided herein is a method of suppressing the proliferation of tumor cells, comprising bringing GM NK cells described herein, into proximity with the tumor cells, e.g., contacting the tumor cells with GM NK cells described herein. Optionally, isolated placental perfusate or isolated placental perfusate cells is brought into proximity with the tumor cells and/or GM NK cells described herein. In another specific alternative, an immunomodulatory compound, e.g., an immunomodulatory compound described above, or thalidomide is additionally brought into proximity with the tumor cells and/or GM NK cells described herein, such that proliferation of the tumor cells is detectably reduced compared to tumor cells of the same type not brought into proximity with GM NK cells described herein. Optionally, isolated placental perfusate or isolated placental perfusate cells are brought into proximity with the tumor cells and/or GM NK cells described herein that have been contacted or brought into proximity with an immunomodulatory compound.

As used herein, in certain alternatives, “contacting,” or “bringing into proximity,” with respect to cells, in one alternative encompasses direct physical, e.g., cell-cell, contact between natural killer cells, e.g., GM NK cell populations described herein, and the tumor cells. In another alternative, “contacting” encompasses presence in the same physical space, e.g., natural killer cells, e.g., GM NK cells described herein, and/or isolated combined natural killer cells are placed in the same container (e.g., culture dish, multiwell plate) as tumor cells. In another alternative, “contacting” natural killer cells, e.g., GM NK cells described herein, and tumor cells is accomplished, e.g., by injecting or infusing the natural killer cells, e.g., GM NK cells, into an individual, e.g., a human comprising tumor cells, e.g., a cancer patient. “Contacting,” in the context of immunomodulatory compounds and/or thalidomide, means, e.g., that the cells and the immunomodulatory compound and/or thalidomide are directly physically contacted with each other, or are placed within the same physical volume (e.g., a cell culture container or an individual).

In a specific alternative, the tumor cells are blood cancer cells, e.g., leukemia cells or lymphoma cells. In more specific alternatives, the cancer is an acute leukemia, e.g., acute T cell leukemia cells, acute myelogenous leukemia (AML) cells, acute promyelocytic leukemia cells, acute myeloblastic leukemia cells, acute megakaryoblastic leukemia cells, precursor B acute lymphoblastic leukemia cells, precursor T acute lymphoblastic leukemia cells, Burkitt's leukemia (Burkitt's lymphoma) cells, or acute biphenotypic leukemia cells; chronic leukemia cells, e.g., chronic myeloid lymphoma cells, chronic myelogenous leukemia (CML) cells, chronic monocytic leukemia cells, chronic lymphocytic leukemia (CLL)/Small lymphocytic lymphoma cells, or B-cell prolymphocytic leukemia cells; hairy cell lymphoma cells; T-cell prolymphocytic leukemia cells; or lymphoma cells, e.g., histiocytic lymphoma cells, lymphoplasmacytic lymphoma cells (e.g., Waldenström macroglobulinemia cells), splenic marginal zone lymphoma cells, plasma cell neoplasm cells (e.g., plasma cell myeloma cells, plasmacytoma cells, monoclonal immunoglobulin deposition disease, or a heavy chain disease), extranodal marginal zone B cell lymphoma (MALT lymphoma) cells, nodal marginal zone B cell lymphoma (NMZL) cells, follicular lymphoma cells, mantle cell lymphoma cells, diffuse large B cell lymphoma cells, mediastinal (thymic) large B cell lymphoma cells, intravascular large B cell lymphoma cells, primary effusion lymphoma cells, T cell large granular lymphocytic leukemia cells, aggressive NK cell leukemia cells, adult T cell leukemia/lymphoma cells, extranodal NK/T cell lymphoma—nasal type cells, enteropathy-type T cell lymphoma cells, hepatosplenic T cell lymphoma cells, blastic NK cell lymphoma cells, mycosis fungoides (Sezary syndrome), primary cutaneous CD30-positive T cell lymphoproliferative disorder (e.g., primary cutaneous anaplastic large cell lymphoma or lymphomatoid papulosis) cells, angioimmunoblastic T cell lymphoma cells, peripheral T cell lymphoma—unspecified cells, anaplastic large cell lymphoma cells, Hodgkin lymphoma cells or nodular lymphocyte-predominant Hodgkin lymphoma cells. In another specific alternative, the tumor cells are multiple myeloma cells or myelodysplastic syndrome cells.

In specific alternatives, the tumor cells are solid tumor cells, e.g., carcinoma cells, for example, adenocarcinoma cells, adrenocortical carcinoma cells, colon adenocarcinoma cells, colorectal adenocarcinoma cells, colorectal carcinoma cells, ductal cell carcinoma cells, lung carcinoma cells, thyroid carcinoma cells, nasopharyngeal carcinoma cells, melanoma cells (e.g., malignant melanoma cells), non-melanoma skin carcinoma cells, or unspecified carcinoma cells; desmoid tumor cells; desmoplastic small round cell tumor cells; endocrine tumor cells; Ewing sarcoma cells; germ cell tumor cells (e.g., testicular cancer cells, ovarian cancer cells, choriocarcinoma cells, endodermal sinus tumor cells, germinoma cells, etc.); hepatosblastoma cells; hepatocellular carcinoma cells; neuroblastoma cells; non-rhabdomyosarcoma soft tissue sarcoma cells; osteosarcoma cells; retinoblastoma cells; rhabdomyosarcoma cells; or Wilms tumor cells. In another alternative, the tumor cells are pancreatic cancer cells or breast cancer cells. In other alternatives, the solid tumor cells are acoustic neuroma cells; astrocytoma cells (e.g., grade I pilocytic astrocytoma cells, grade II low-grade astrocytoma cells; grade III anaplastic astrocytoma cells; or grade IV glioblastoma multiforme cells); chordoma cells; craniopharyngioma cells; glioma cells (e.g., brain stem glioma cells; ependymoma cells; mixed glioma cells; optic nerve glioma cells; or subependymoma cells); glioblastoma cells; medulloblastoma cells; meningioma cells; metastatic brain tumor cells; oligodendroglioma cells; pineoblastoma cells; pituitary tumor cells; primitive neuroectodermal tumor cells; or schwannoma cells. In another alternative, the tumor cells are prostate cancer cells.

As used herein, “therapeutically beneficial” and “therapeutic benefits” include, but are not limited to, e.g., reduction in the size of a tumor; lessening or cessation of expansion of a tumor; reducing or preventing metastatic disease; reduction in the number of cancer cells in a tissue sample, e.g., a blood sample, per unit volume; the clinical improvement in any symptom of the particular cancer or tumor said individual has, the lessening or cessation of worsening of any symptom of the particular cancer the individual has, etc.

c. 6.9.3. Treatment of Cancers Using GM NK Cells and Other Anticancer Agents

Providing therapy to an individual having cancer using the GM NK cells described herein, can be part of an anticancer therapy regimen that includes one or more additional anticancer agents. In addition or alternatively, providing therapy to an individual having cancer using the GM NK cells a described herein can be used to supplement an anticancer therapy that includes one or more other anticancer agents. Such anticancer agents are well-known in the art and include anti-inflammatory agents, immumodulatory agents, cytotoxic agents, cancer vaccines, chemotherapeutics, HDAC inhibitors (e.g., HDAC6i (ACY-241)), and siRNAs. Specific anticancer agents that may be administered to an individual having cancer, e.g., an individual having tumor cells, in addition to the GM NK cells described herein, include but are not limited to: acivicin; aclarubicin; acodazole hydrochloride; acronine; adozelesin; adriamycin; adrucil; aldesleukin; altretamine; ambomycin; ametantrone acetate; amsacrine; anastrozole; anthramycin; asparaginase (e.g., from Erwinia chrysan; Erwinaze); asperlin; avastin (bevacizumab); azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; celecoxib (COX-2 inhibitor); Cerubidine; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; Elspar; enloplatin; enpromate; epipropidine; epirubicin hydrochloride; erbulozole; esorubicin hydrochloride; estramustine; estramustine phosphate sodium; etanidazole; etoposide; etoposide phosphate; Etopophos; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; Idamycin; idarubicin hydrochloride; ifosfamide; ilmofosine; iproplatin; irinotecan; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; Proleukin; Purinethol; puromycin; puromycin hydrochloride; pyrazofurin; Rheumatrex; riboprine; safingol; safingol hydrochloride; semustine; simtrazene; sparfosate sodium; sparsomycin; spirogermanium hydrochloride; spiromustine; spiroplatin; streptonigrin; streptozocin; sulofenur; Tabloid; talisomycin; tecogalan sodium; taxotere; tegafur; teloxantrone hydrochloride; temoporfin; teniposide; teroxirone; testolactone; thiamiprine; thioguanine; thiotepa; tiazofurin; tirapazamine; Toposar; toremifene citrate; trestolone acetate; Trexall; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and/or zorubicin hydrochloride.

Additional anti-cancer drugs, which can be provided in some contemplated methods involving GM NK cells include, but are not limited to: 20-epi-1,25 dihydroxyvitamin D3; 5-azacytidine; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptosar (also called Campto; irinotecan) camptothecin derivatives; capecitabine; carboxamide-amino-triazole; carboxyamidotriazole; CaRest M3; CARN 700; cartilage derived inhibitor; carzelesin; casein kinase inhibitors (ICOS); castanospermine; CC-122; CC-220; CC-486; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidenmin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; doxorubicin; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine (e.g., Fludara); fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imatinib (e.g., GLEEVEC®), imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mitoguazone; mitolactol; mitomycin analogues; mitonafide; mitotoxin fibroblast growth factor-saporin; mitoxantrone; mofarotene; molgramostim; anti-EGFR antibody (e.g., Erbitux (cetuximab)); anti-CD19 antibody; anti-CD20 antibody (e.g., rituximab); anti-CS-1 antibody (e.g., elotuzumab (BMS/AbbVie)); anti-CD38 antibody (e.g., daratumumab (Genmab/Janssen Biotech); anti-CD138 antibody (e.g., indatuximab (Biotest AG Dreieich)); anti-PD-1 antibody; anti-PD-L1 antibody (e.g., durvalumab (AstraZeneca)); anti-NKG2A antibody (e.g., monalizumab (IPH2201; Innate Pharma)); anti-DLL4 antibody (e.g., demcizumab (Oncomed/Celgene)); anti-DLL4 and anti-VEGF bispecific antibody; anti-RSPO3 antibody; anti-TIGIT antibody; ICOS agonist antibody; anti-disialoganglioside (GD2) antibody (e.g., monoclonal antibody 3F8 or ch14.18); anti-ErbB2 antibody (e.g., herceptin); human chorionic gonadotrophin; monophosphoryl lipid A+myobacterium cell wall sk; mopidamol; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; nilutamide; nisamycin; nitric oxide modulators; nitroxide antioxidant; nitrullyn; oblimersen (GENASENSE®); O⁶-benzylguanine; octreotide; okicenone; oligonucleotides; onapristone; ondansetron; ondansetron; oracin; oral cytokine inducer; ormaplatin; osaterone; oxaliplatin (e.g., Floxatin); oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; rafantagonists; raltitrexed; ramosetron; rasfarnesyl protein transferase inhibitors; rasinhibitors; ras-GAP inhibitor; retelliptine demethylated; rhenium Re 186 etidronate; rhizoxin; ribozymes; RII retinamide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stipiamide; stromelysin inhibitors; sulfinosine; superactive vasoactive intestinal peptide antagonist; suradista; suramin; swainsonine; tallimustine; tamoxifen methiodide; tauromustine; tazarotene; tecogalan sodium; tegafur; tellurapyrylium; telomerase inhibitors; temoporfin; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; Vectibix (panitumumab)velaresol; veramine; verdins; verteporfin; vinorelbine; vinxaltine; vitaxin; vorozole; Welcovorin (leucovorin); Xeloda (capecitabine); zanoterone; zeniplatin; zilascorb; and/or zinostatin stimalamer.

Therapy provided to an individual having cancer using the GM NK cells described herein can be part of an anticancer therapy regimen that includes one or more immune checkpoint modulators. In certain alternatives, the immune checkpoint modulator modulates an immune checkpoint molecule such as CD28, OX40, Glucocorticoid-Induced Tumour-necrosis factor Receptor-related protein (GITR), CD137 (4-1BB), CD27, Herpes Virus Entry Mediator (HVEM), T cell Immunoglobulin and Mucin-domain containing-3 (TIM-3), Lymphocyte-Activation Gene 3 (LAG-3), Cytotoxic T-Lymphocyte-associated Antigen-4 (CTLA-4), V-domain Immunoglobulin Suppressor of T cell Activation (VISTA), B and T Lymphocyte Attenuator (BTLA), PD-1, and/or PD-L1. In certain alternatives, the immune checkpoint molecule is an antibody or antigen-binding fragment thereof. In certain alternatives, the immune checkpoint modulator is an agonist of an immune checkpoint molecule. In certain alternatives, the immune checkpoint molecule is CD28, OX40, Glucocorticoid-Induced Tumour-necrosis factor Receptor-related protein (GITR), CD137 (4-1BB), CD27, ICOS (CD278); Inducible T-cell Costimulator) and/or Herpes Virus Entry Mediator (HVEM). In certain alternatives, the immune checkpoint modulator is an antibody or antigen-binding fragment thereof. In certain alternatives, the immune checkpoint modulator is an antagonist of an immune checkpoint molecule. In certain alternatives, the immune checkpoint molecule is T cell Immunoglobulin and Mucin-domain containing-3 (TIM-3), Lymphocyte-Activation Gene 3 (LAG-3), Cytotoxic T-Lymphocyte-associated Antigen-4 (CTLA-4), V-domain Immunoglobulin Suppressor of T cell Activation (VISTA), B and T Lymphocyte Attenuator (BTLA), PD-1, and/or PD-L1. In certain alternatives, the immune checkpoint modulator is an antibody or antigen-binding fragment thereof. In certain alternatives, the immune checkpoint modulator is an antibody or antigen-binding fragment thereof. In certain alternatives, the antibody or antibody-binding fragment thereof binds PD-1. In certain alternatives, the antibody or antibody-binding fragment thereof that binds PD-1 is nivolumab (OPDIVO®′ BMS-936558, MDX-1106, ONO-4538; Bristol-Myers Squibb, Ono Pharmaceuticals, Inc.), pembrolizumab (KEYTRUDA®, lambrolizumab, MK-3475; Merck), pidilizumab (CT-011; Curetech, Medivation); MEDI0680 (AMP-514; MedImmune, AstraZeneca); PDR-001 (Novartis), SHR1210, or INCSHR1210; Incyte, Jiangsu Hengrui). In certain alternatives, the antibody or antigen-binding fragment thereof binds PD-L1. In certain alternatives, the antibody or antigen-binding fragment thereof that binds PD-L1 is durvalumab (MEDI4736; MedImmune, AstraZeneca), BMS-936559 (MDX-1105; Bristol-Myers Squibb), avelumab (MSB0010718C; Merck Serono, Pfizer), or atezolizumab (MPDL-3280A; Genentech, Roche). In certain alternatives, the antibody or antibody-binding fragment thereof binds LAG-3. In certain alternatives, the antibody or antibody-binding fragment thereof that binds LAG-3 is BMS-986016 (Bristol-Myers Squibb), GSK2831781 (GlaxoSmithKline), or LAG525 (Novartis). In certain alternatives, the antibody or antibody-binding fragment thereof binds CTLA-4. In certain alternatives, the antibody or antibody-binding fragment thereof that binds CTLA-4 is ipilimumab (YERVOY™, BMS-734016, MDX010, MDX-101; Bristol-Myers Squibb), or tremelimumab (CP-675,206; MedImmune, AstraZeneca). In certain alternatives, the antibody or antibody-binding fragment thereof binds OX40. In certain alternatives, the antibody or antibody-binding fragment thereof that binds OX40 is MEDI6469 (MedImmune, AstraZeneca), MEDI0562 (MedImmune, AstraZeneca), or KHK4083 (Kyowa Hakko Kirin). In certain alternatives, the antibody or antibody-binding fragment thereof binds GITR. In certain alternatives, the antibody or antibody-binding fragment thereof that binds GITR is TRX518 (Leap Therapeutics) or MEDI1873 (MedImmune, AstraZeneca). In certain alternatives, the antibody or antibody-binding fragment thereof binds CD137 (4-1BB). In certain alternatives, the antibody or antibody-binding fragment thereof that binds CD137 (4-1BB) is PF-2566 (PF-05082566; Pfizer), or urelumab (BMS-663513; Bristol-Myers Squibb). In certain alternatives, the antibody or antibody-binding fragment thereof binds CD27. In certain alternatives, the antibody or antibody-binding fragment thereof that binds CD27 is varilumab (CDX-1127; Celldex Therapies).

In certain alternatives, therapy for an individual having cancer using the GM NK cells described herein is part of an anticancer therapy regimen that includes lenalidomide or pomalidomide. In certain alternatives, therapy of an individual having cancer using the GM NK cells described herein is part of an anticancer therapy regimen that includes an HDAC inhibitor. In certain alternatives, therapy of an individual having cancer using the GM NK described herein is part of an anticancer therapy regimen that includes an anti-CS-1 antibody. In certain alternatives, therapy of an individual having cancer using the GM NK cells described herein is part of an anticancer therapy regimen that includes an anti-CD38 antibody. In certain alternatives, therapy of an individual having cancer using the GM NK cells described herein is part of an anticancer therapy regimen that includes an anti-CD138 antibody. In certain alternatives, therapy of an individual having cancer using the GM NK cells described herein, is part of an anticancer therapy regimen that includes an anti-PD-1 antibody. In certain alternatives, therapy of an individual having cancer using the GM NK described herein is part of an anticancer therapy regimen that includes an anti-PD-L1 antibody. In certain alternatives, therapy of an individual having cancer using the GM NK cells described herein is part of an anticancer therapy regimen that includes an anti-NKG2A antibody. In certain alternatives, therapy of an individual having cancer using the GM NK cells described herein is part of an anticancer therapy regimen that includes an anti-CD20 antibody (e.g., rituximab; RITUXAN®). In certain alternatives, therapy of an individual having cancer using the GM NK cells described herein is part of an anticancer therapy regimen that includes CC-122. In certain alternatives, therapy of an individual having cancer using the GM NK cells described herein is part of an anticancer therapy regimen that includes CC-220. In certain alternatives, therapy of an individual having cancer using the GM NK cells described herein is part of an anticancer therapy regimen that includes an anti-DLL4 antibody (e.g., demcizumab). In certain alternatives, therapy of an individual having cancer using the GM NK cells described herein is part of an anticancer therapy regimen that includes an anti-DLL4 and anti-VEGF bispecific antibody. In certain alternatives, therapy of an individual having cancer using the GM NK cells described herein is part of an anticancer therapy regimen that includes an anti-RSPO3 antibody. In certain alternatives, therapy of an individual having cancer using the GM NK cells described herein is part of an anticancer therapy regimen that includes an anti-TIGIT antibody. In certain alternatives, therapy of an individual having cancer using the GM NK cells described herein is part of an anticancer therapy regimen that includes an ICOS agonist antibody.

In some alternatives, therapy of an individual having cancer using the GM NK cells described herein is part of an anticancer therapy regimen for antibody-dependent cell-mediated cytotoxicity (ADCC). In one alternative, the ADCC regimen comprises administration of one or more antibodies (e.g., an antibody described in the foregoing paragraph) in combination with GM NK cells described herein. Several types of cancer can be inhibited or treated using such ADCC methods, including but not limited to acute lymphoblastic leukemia (ALL) or other B-cell malignancies (lymphomas and leukemias), neuroblastoma, melanoma, breast cancers, and head and neck cancers. In specific alternatives, the ADCC therapy comprises administration of one or more of the following antibodies anti-EGFR antibody (e.g., Erbitux (cetuximab)), anti-CD19 antibody, anti-CD20 antibody (e.g., rituximab), anti-disialoganglioside (GD2) antibody (e.g., monoclonal antibody 3F8 or ch14.18), or anti-ErbB2 antibody (e.g., herceptin), in combination with GM NK cells described herein. In one alternative, the ADCC regimen comprises administration of an anti-CD33 antibody in combination with GM NK cells described herein. In one alternative, the ADCC regimen comprises administration of an anti-CD20 antibody in combination with GM NK cells described herein. In one alternative, the ADCC regimen comprises administration of an anti-CD138 antibody in combination with GM NK cells described herein. In one alternative, the ADCC regimen comprises administration of an anti-CD32 antibody in combination with GM NK cells described herein.

d. 6.9.4. Treatment of Viral Infection

In another alternative, provided herein is a method of providing therapy of an individual having a viral infection, comprising administering to said individual a therapeutically effective amount of GM NK cells described herein. In certain alternatives, the individual has a deficiency of natural killer cells, e.g., a deficiency of NK cells or other innate lymphoid cells active against the individual's viral infection. In certain specific alternatives, the GM NK cells described herein are contacted or brought into proximity with an immunomodulatory compound, e.g., an immunomodulatory compound above, or thalidomide, prior to said administration. In certain other specific alternatives, said administering comprises administering an immunomodulatory compound, e.g., an immunomodulatory compound described above, or thalidomide, to said individual in addition to said GM NK cells described herein, wherein said amount is an amount that, e.g., results in a detectable improvement of, lessening of the progression of, or elimination of, one or more symptoms of said viral infection. In specific alternatives, the viral infection is an infection by a virus of the Adenoviridae, Picornaviridae, Herpesviridae, Hepadnaviridae, Flaviviridae, Retroviridae, Orthomyxoviridae, Paramyxoviridae, Papilommaviridae, Rhabdoviridae, or Togaviridae family. In more specific alternatives, said virus is human immunodeficiency virus (HIV), coxsackievirus, hepatitis A virus (HAV), poliovirus, Epstein-Barr virus (EBV), herpes simplex type 1 (HSV1), herpes simplex type 2 (HSV2), human cytomegalovirus (CMV), human herpesvirus type 8 (HHV8), herpes zoster virus (varicella zoster virus (VZV) or shingles virus), hepatitis B virus (HBV), hepatitis C virus (HCV), hepatitis D virus (HDV), hepatitis E virus (HEV), influenza virus (e.g., influenza A virus, influenza B virus, influenza C virus, or thogotovirus), measles virus, mumps virus, parainfluenza virus, papillomavirus, rabies virus, or rubella virus.

In other more specific alternatives, said virus is adenovirus species A, serotype 12, 18, or 31; adenovirus species B, serotype 3, 7, 11, 14, 16, 34, 35, or 50; adenovirus species C, serotype 1, 2, 5, or 6; species D, serotype 8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, or 51; species E, serotype 4; or species F, serotype 40 or 41.

In certain other more specific alternatives, the virus is Apoi virus (APOIV), Aroa virus (AROAV), bagaza virus (BAGV), Banzi virus (BANV), Bouboui virus (BOUV), Cacipacore virus (CPCV), Carey Island virus (CIV), Cowbone Ridge virus (CRV), Dengue virus (DENV), Edge Hill virus (EHV), Gadgets Gully virus (GGYV), Ilheus virus (ILHV), Israel turkey meningoencephalomyelitis virus (ITV), Japanese encephalitis virus (JEV), Jugra virus (JUGV), Jutiapa virus (JUTV), kadam virus (KADV), Kedougou virus (KEDV), Kokobera virus (KOKV), Koutango virus (KOUV), Kyasanur Forest disease virus (KFDV), Langat virus (LGTV), Meaban virus (MEAV), Modoc virus (MODV), Montana myotis leukoencephalitis virus (MMLV), Murray Valley encephalitis virus (MVEV), Ntaya virus (NTAV), Omsk hemorrhagic fever virus (OHFV), Powassan virus (POWV), Rio Bravo virus (RBV), Royal Farm virus (RFV), Saboya virus (SABV), St. Louis encephalitis virus (SLEV), Sal Vieja virus (SVV), San Perlita virus (SPV), Saumarez Reef virus (SREV), Sepik virus (SEPV), Tembusu virus (TMUV), tick-borne encephalitis virus (TBEV), Tyuleniy virus (TYUV), Uganda S virus (UGSV), Usutu virus (USUV), Wesselsbron virus (WESSV), West Nile virus (WNV), Yaounde virus (YAOV), Yellow fever virus (YFV), Yokose virus (YOKV), or Zika virus (ZIKV).

In other alternatives, the GM NK cells described herein are administered to an individual having a viral infection as part of an antiviral therapy regimen that includes one or more other antiviral agents. In some alternatives, the individual has been selected to receive genetically modified NK cells an antiviral agents. Specific antiviral agents that may be administered to an individual having a viral infection include, but are not limited to: imiquimod, podofilox, podophyllin, interferon alpha (IFNα), reticolos, nonoxynol-9, acyclovir, famciclovir, valaciclovir, ganciclovir, cidofovir; amantadine, rimantadine; ribavirin; zanamavir and oseltaumavir; protease inhibitors such as indinavir, nelfinavir, ritonavir, or saquinavir; nucleoside reverse transcriptase inhibitors such as didanosine, lamivudine, stavudine, zalcitabine, or zidovudine; and non-nucleoside reverse transcriptase inhibitors such as nevirapine, or efavirenz.

e. 6.9.5. Administration

Administration of an isolated population of GM NK cells or a pharmaceutical composition thereof may be systemic or local. In specific alternatives, administration is parenteral. In specific alternatives, administration of an isolated population of GM NK cells or a pharmaceutical composition thereof to a subject is by injection, infusion, intravenous (IV) administration, intrafemoral administration, or intratumoral administration. In specific alternatives, administration of an isolated population of GM NK cells or a pharmaceutical composition thereof to a subject is performed with a device, a matrix, or a scaffold. In specific alternatives, administration an isolated population of GM NK cells or a pharmaceutical composition thereof to a subject is by injection. In specific alternatives, administration an isolated population of GM NK cells or a pharmaceutical composition thereof to a subject is via a catheter. In specific alternatives, the injection of GM NK cells is a local injection. In more specific alternatives, the local injection is directly into a solid tumor (e.g., a sarcoma). In specific alternatives, administration of an isolated population of GM NK cells or a pharmaceutical composition thereof to a subject is by injection by syringe. In specific alternatives, administration of an isolated population of GM NK cells or a pharmaceutical composition thereof to a subject is via guided delivery. In specific alternatives, administration of an isolated population of GM NK cells or a pharmaceutical composition thereof to a subject by injection is aided by laparoscopy, endoscopy, ultrasound, computed tomography, magnetic resonance, or radiology.

i. 6.9.5.1. Administration of Cells

In certain alternatives, GM NK cells described herein are used, e.g., administered to an individual, in any amount or number that results in a detectable therapeutic benefit to the individual, e.g., an effective amount, wherein the individual has a viral infection, cancer, or tumor cells, for example, an individual having tumor cells, a solid tumor or a blood cancer, e.g., a cancer patient. Such cells can be administered to such an individual by absolute numbers of cells, e.g., said individual can be administered at, at least, or at most, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ GM NK cells described herein or any number of cells in between a range defined by any two of the aforementioned values. In other alternatives, GM NK cells described herein can be administered to such an individual by relative numbers of cells, e.g., said individual can be administered at, at least, or at most, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, or 1×10¹¹ GM NK cells described herein per kilogram of the individual or any number of cells per kilogram of the individual in between a range defined by any two of the aforementioned values. In other alternatives, GM NK cells described herein can be administered to such an individual by relative numbers of cells, e.g., said individual can be administered at, at least, or at most, 1×10⁵, 5×10⁵, 1×10⁶, 5×10⁶, 1×10⁷, 5×10⁷, 1×10⁸, or 5×10⁸ GM NK cells described herein per kilogram of the individual or any number of cells per kilogram of the individual in between a range defined by any two of the aforementioned values. GM NK cells described herein can be administered to such an individual according to an approximate ratio between a number of GM NK cells and a number of tumor cells in said individual (e.g., an estimated number). For example, GM NK cells described herein can be administered to said individual in a ratio of, at least or at most 1:1, 1:1, 3:1, 4:1, 5:1, 6:1, 7:1, 8:1, 9:1, 10:1, 15:1, 20:1, 25:1, 30:1, 35:1, 40:1, 45:1, 50:1, 55:1, 60:1, 65:1, 70:1, 75:1, 80:1, 85:1, 90:1, 95:1 or 100:1 to the number of tumor cells in the individual or a ratio of GM NK cells to the number of tumor cells in the individual that is in between a range defined by any two of the aforementioned ratios. The number of tumor cells in such an individual can be estimated, e.g., by counting the number of tumor cells in a sample of tissue from the individual, e.g., blood sample, biopsy, or the like. In specific alternatives, e.g., for solid tumors, said counting is performed in combination with imaging of the tumor or tumors to obtain an approximate tumor volume. In a specific alternative, an immunomodulatory compound or thalidomide, e.g., an effective amount of an immunomodulatory compound or thalidomide, are administered to the individual in addition to the GM NK cells described herein.

In certain alternatives, the method of suppressing the proliferation of tumor cells, e.g., in an individual; therapy of an individual having a deficiency in the individual's natural killer cells; or therapy of an individual having a viral infection; or therapy of an individual having cancer, e.g., an individual having tumor cells, a blood cancer or a solid tumor, comprises bringing the tumor cells into proximity with, or administering to said individual, a combination of GM NK cells and one or more of placental perfusate and/or placental perfusate cells. In specific alternatives, the method additionally comprises bringing the tumor cells into proximity with, or administering to the individual, an immunomodulatory compound or thalidomide.

In a specific alternative, for example, therapy of an individual having a deficiency in the individual's natural killer cells (e.g., a deficiency in the number of NK cells or in the NK cells' reactivity to a cancer, tumor or virally-infected cells); or therapy of an individual having a cancer or a viral infection, or suppression of tumor cell proliferation, comprises bringing said tumor cells into proximity with, or administering to said individual, GM NK cells described herein supplemented with isolated placental perfusate cells or placental perfusate. In specific alternatives, 1×10⁴, 5×10⁴, 1×10⁵, 5×10⁵, 1×10⁶, 5×10⁶, 1×10⁷, 5×10⁷, 1×10⁸, 5×10⁸ or more NK cells are produced using the methods described herein per milliliter or any number of cells per milliliter in between a range defined by any two of the aforementioned values are produced, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more GM NK cells or any number of GM NK cells in between a range defined by any two of the aforementioned values are produced using the methods described herein are supplemented with, or at least, 1×10⁴, 5×10⁴, 1×10⁵, 5×10⁵, 1×10⁶, 5×10⁶, 1×10⁷, 5×10⁷, 1×10⁸, 5×10⁸ or more isolated placental perfusate cells per milliliter, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more isolated placental perfusate cells. In other more specific alternatives, about 1×10⁴, 5×10⁴, 1×10⁵, 5×10⁵, 1×10⁶, 5×10⁶, 1×10⁷, 5×10⁷, 1×10⁸, 5×10⁸ or more GM NK cells or any number of GM NK cells in between a range defined by any two of the aforementioned values are produced using the methods described herein or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more GM NK cells or any number of GM NK cells in between a range defined by any two of the aforementioned values are produced using the methods described herein are supplemented with, or at least, 1, 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, 800, 850, 900, 950 or 1000 mL of perfusate, or 1 unit of perfusate.

In another specific alternative, therapy of an individual having a deficiency in the individual's natural killer cells; therapy of an individual having cancer; therapy of an individual having a viral infection; or suppression of tumor cell proliferation, comprises bringing the tumor cells into proximity with, or administering to the individual, GM NK cells described herein, wherein said cells are supplemented with adherent placental cells, e.g., adherent placental stem cells or multipotent cells, e.g., CD34⁻, CD10⁺, CD105⁺, CD200⁺ tissue culture plastic-adherent placental cells. In specific alternatives, the GM NK cells described herein are supplemented with 1×10⁴, 5×10⁴, 1×10⁵, 5×10⁵, 1×10⁶, 5×10⁶, 1×10⁷, 5×10⁷, 1×10⁸, 5×10⁸ or more adherent placental stem cells per milliliter or any number of adherent placental stem cells per milliliter in between a range defined by any two of the aforementioned values, or 1×10⁴, 5×10⁴, 1×10⁵, 5×10⁵, 1×10⁶, 5×10⁶, 1×10⁷, 5×10⁷, 1×10⁸, 5×10⁸, 1×10⁹, 5×10⁹, 1×10¹⁰, 5×10¹⁰, 1×10¹¹ or more adherent placental cells or any number of adherent placental stem cells per milliliter in between a range defined by any two of the aforementioned values, e.g., adherent placental stem cells or multipotent cells.

In another specific alternative, therapy of an individual having a deficiency in the individual's natural killer cells; therapy of an individual having cancer; therapy of an individual having a viral infection; or suppression of tumor cell proliferation, is performed using an immunomodulatory compound or thalidomide in combination with GM NK cells described herein, wherein said cells are supplemented with conditioned medium, e.g., medium conditioned by CD34⁻, CD10⁺, CD105⁺, CD200⁺ tissue culture plastic-adherent placental cells, e.g., 0.1, 0.2, 0.3, 0.4, 0.5, 0.6, 0.1, 0.8, 0.9, 1, 2, 3, 4, 5, 6, 7, 8, 9, 10 mL of stem cell-conditioned culture medium per unit of perfusate or any volume in between a range defined by any two of the aforementioned values, or per 10⁴, 10⁵, 10⁶, 10⁷, 10⁸, 10⁹, 10¹⁰, or 10¹¹ GM NK cells described herein or any number of GM NK cells in between a range defined by any two of the aforementioned values. In certain alternatives, the tissue culture plastic-adherent placental cells are the multipotent adherent placental cells described in U.S. Pat. Nos. 7,468,276 and 8,057,788, the disclosures of which are incorporated herein by reference in their entireties. In another specific alternative, the method additionally comprises bringing the tumor cells into proximity with, or administering to the individual, an immunomodulatory compound or thalidomide.

In another specific alternative, therapy of an individual having a deficiency in the individual's natural killer cells; therapy of an individual having cancer; therapy of an individual having a viral infection; or suppression of tumor cell proliferation, in which said GM NK cells described herein are supplemented with placental perfusate cells, the perfusate cells are brought into proximity with interleukin-2 (IL-2) for a period of time prior to said bringing into proximity. In certain alternatives, said period of time is, at least, or at most 1, 2, 3, 4, 5, 6, 7, 8, 9, 10, 12, 14, 16, 18, 20, 22, 24, 26, 28, 30, 32, 34, 36, 38, 40, 42, 44, 46 or 48 hours prior to said bringing into proximity or any number of hours in between a range defined by any two of the aforementioned values.

The GM NK cells described herein and optionally perfusate or perfusate cells, can be administered once to an individual having a viral infection, an individual having cancer, or an individual having tumor cells, during a course of anticancer therapy; or can be administered multiple times, e.g., once every 1, 2, 3, 4, 5, 6, 7, 8, 9, 10, 11, 12, 13, 14, 15, 16, 17, 18, 19, 20, 21, 22 or 23 hours, or once every 1, 2, 3, 4, 5, 6 or 7 days, or once every 1, 2, 3, 4, 5, 6, 7, 8, 9, 10, 24, 36 or more weeks during therapy. In some alternatives, the GM NK cells are administered once to an individual having a viral infection, an individual having cancer, or an individual having tumor cells, during a course of anticancer therapy every 1, 2, 3, 4, 5, 6, 7, 8, 9, 10, 11, 12, 13, 14, 15, 16, 17, 18, 19, 20, 21, 22 or 23 hours or any amount of time in between a range defined by any two of the aforementioned values. In some alternatives, the GM NK cells are administered once to an individual having a viral infection, an individual having cancer, or an individual having tumor cells, during a course of anticancer therapy every 1, 2, 3, 4, 5, 6 or 7 days or any amount of time in between a range defined by any two of the aforementioned values. In some alternatives, the GM NK cells are administered once to an individual having a viral infection, an individual having cancer, or an individual having tumor cells, during a course of anticancer therapy once every 1, 2, 3, 4, 5, 6, 7, 8, 9, 10, 24, 36 or more weeks during therapy or any amount of time in between a range defined by any two of the aforementioned values. In alternatives in which cells and an immunomodulatory compound or thalidomide are used, the immunomodulatory compound or thalidomide, and cells or perfusate, are administered to the individual together, e.g., in the same formulation; separately, e.g., in separate formulations, at approximately the same time; or can be administered separately, e.g., on different dosing schedules or at different times of the day. Similarly, in alternatives in which cells and an antiviral compound or anticancer compound are used, the antiviral compound or anticancer compound, and cells or perfusate, can be administered to the individual together, e.g., in the same formulation; separately, e.g., in separate formulations, at approximately the same time; or can be administered separately, e.g., on different dosing schedules or at different times of the day. The GM NK cells described herein and perfusate or perfusate cells, can be administered without regard to whether GM NK cells described herein, perfusate, or perfusate cells have been administered to the individual in the past.

10. KITS

Provided herein is a pharmaceutical pack or kit comprising one or more containers filled with one or more of the compositions described herein, e.g., a composition comprising one or more populations of GM NK cells. Optionally associated with such container(s) can be a notice in the form prescribed by a governmental agency regulating the manufacture, use or sale of pharmaceuticals or biological products, which notice reflects approval by the agency of manufacture, use or sale for human administration.

The kits encompassed herein can be used in accordance with the methods described herein, e.g., methods of suppressing the growth of tumor cells and/or methods of treating cancer, e.g., hematologic cancer, and/or methods of treating viral infection. In one alternative, a kit comprises GM NK cells described herein or a composition thereof, in one or more containers. In a specific alternative, provided herein is a kit comprising one or more NK cell populations described herein, or a composition thereof.

11. More Alternatives

In some alternatives, a population of natural killer cells, wherein the natural killer (NK) cells are genetically modified such that they lack expression of an NK inhibitory molecule or manifest reduced expression of an NK inhibitory molecule is provided. In some alternatives, the NK inhibitory molecule is CBLB, NKG2A and/or TGFBR2. In some alternatives, the NK inhibitory molecule is CBLB. In some alternatives, the CBLB expression has been knocked out. In some alternatives, the CBLB expression has been knocked out by CRISPR/CAS9 system, a zinc finger nuclease or TALEN nuclease. In some alternatives, the CBLB expression has been knocked out by a CRISPR-related technique. In some alternatives, the knockout of CBLB expression results in NK cells with higher cytotoxicity against tumor cells than NK cells wherein CBLB has not been knocked out. In some alternatives, the tumor cells are multiple myeloma cells. In some alternatives, the tumor cells are RPMI8226 cells. In some alternatives, the tumor cells are U266 cells. In some alternatives, the tumor cells are ARH77 cells. In some alternatives, the tumor cells are acute myeloid leukemia cells. In some alternatives, the tumor cells are HL60 cells. In some alternatives, the tumor cells are KG1 cells. In some alternatives, the knockout of CBLB expression results in NK cells with higher IFNγ secretion when stimulated with ICAM-1 and MICA than NK cells wherein CBLB has not been knocked out. In some alternatives, the knockout of CBLB expression results in NK cells with higher degranulation when stimulated with ICAM-1 and MICA than NK cells wherein CBLB has not been knocked out. In some alternatives, the degranulation is measured by an increase in CD107a. In some alternatives, the knockout of CBLB expression results in NK cells with a change in the secretion of one or more of GM-CSF, soluble CD137 (sCD137), IFNγ, MIP1α, MIP1β, TNFα or perform when co-cultured with multiple myeloma cells, compared to NK cells wherein CBLB has not been knocked out. In some alternatives, the NK inhibitory molecule is NKG2A. In some alternatives, the NKG2A expression has been knocked out. In some alternatives, the NKG2A expression has been knocked out by CRISPR/CAS9 system, a zinc finger nuclease or TALEN nuclease. In some alternatives, the NKG2A expression has been knocked out by a CRISPR-related technique. In some alternatives, the knockout of NKG2A expression results in NK cells with higher cytotoxicity against tumor cells than NK cells wherein NKG2A has not been knocked out. In some alternatives, the tumor cells are multiple myeloma cells. In some alternatives, the tumor cells are RPMI8226 cells. In some alternatives, the tumor cells are U266 cells. In some alternatives, the tumor cells are ARH77 cells. In some alternatives, the knockout of NKG2A expression results in NK cells with higher degranulation when stimulated with ICAM-1 and MICA in the presence of an NKG2A agonist antibody than NK cells wherein NKG2A has not been knocked out. In some alternatives, the degranulation is measured by an increase in CD107a. In some alternatives, the knockout of NKG2A expression results in NK cells with a change in the secretion of one or more of GM-CSF, soluble CD137 (sCD137), IFNγ, MIP1α, MIP1β, TNFα or perform, compared to NK cells wherein NKG2A has not been knocked out. In some alternatives, the NK inhibitory molecule is TGFBR2. In some alternatives, the TGFBR2 expression has been knocked out. In some alternatives, the TGFBR2 expression has been knocked out by CRISPR/CAS9 system, a zinc finger nuclease or TALEN nuclease. In some alternatives, the TGFBR2 expression has been knocked out by a CRISPR-related technique. In some alternatives, the knockout of TGFBR2 expression results in resistance to TGFβ mediated inhibition of NK cell cytotoxicity against tumor cells compared to NK cells wherein TGFBR2 has not been knocked out. In some alternatives, the tumor cells are multiple myeloma cells. In some alternatives, the tumor cells are RPMI8226 cells. In some alternatives, the tumor cells are acute myeloid leukemia cells. In some alternatives, the tumor cells are K562 cells. In some alternatives, the tumor cells are chronic myeloid leukemia cells. In some alternatives, the tumor cells are HL-60 cells. In some alternatives, the NK cells are placenta derived (PNK cells). In some alternatives, the natural killer cells are CD56⁺CD3−CD117⁺CD11a⁺, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives of the method, said natural killer cells additionally express T-bet, GZMB, NKp46, NKp30, and NKG2D. In some alternatives, said natural killer cells express CD94. In some alternatives, said natural killer cells do not express CD94.

In some alternatives, a population of natural killer cells, wherein the natural killer (NK) cells are genetically modified to comprise a modified CD16 is provided. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the CD16 has a proline at position 197 of CD16a. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the NK cells are placenta derived (PNK cells). In some alternatives, the natural killer cells are CD56⁺CD3−CD117⁺CD11a⁺, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives of the method, said natural killer cells additionally express T-bet, GZMB, NKp46, NKp30, and NKG2D. In some alternatives, said natural killer cells express CD94. In some alternatives, said natural killer cells do not express CD94.

In some alternatives, a method of suppressing the proliferation of tumor cells comprising contacting the tumor cells with natural killer cells from the population of any one of the alternatives herein. In some alternatives, the natural killer (NK) cells are genetically modified such that they lack expression of an NK inhibitory molecule or manifest reduced expression of an NK inhibitory molecule is provided. In some alternatives, the NK inhibitory molecule is CBLB, NKG2A and/or TGFBR2. In some alternatives, the NK inhibitory molecule is CBLB. In some alternatives, the CBLB expression has been knocked out. In some alternatives, the CBLB expression has been knocked out by CRISPR/CAS9 system, a zinc finger nuclease or TALEN nuclease. In some alternatives, the CBLB expression has been knocked out by a CRISPR-related technique. In some alternatives, the knockout of CBLB expression results in NK cells with higher cytotoxicity against tumor cells than NK cells wherein CBLB has not been knocked out. In some alternatives, the tumor cells are multiple myeloma cells. In some alternatives, the tumor cells are RPMI8226 cells. In some alternatives, the tumor cells are U266 cells. In some alternatives, the tumor cells are ARH77 cells. In some alternatives, the tumor cells are acute myeloid leukemia cells. In some alternatives, the tumor cells are HL60 cells. In some alternatives, the tumor cells are KG1 cells. In some alternatives, the knockout of CBLB expression results in NK cells with higher IFNγ secretion when stimulated with ICAM-1 and MICA than NK cells wherein CBLB has not been knocked out. In some alternatives, the knockout of CBLB expression results in NK cells with higher degranulation when stimulated with ICAM-1 and MICA than NK cells wherein CBLB has not been knocked out. In some alternatives, the degranulation is measured by an increase in CD107a. In some alternatives, the knockout of CBLB expression results in NK cells with a change in the secretion of one or more of GM-CSF, soluble CD137 (sCD137), IFNγ, MIP1α, MIP1β, TNFα or perform when co-cultured with multiple myeloma cells, compared to NK cells wherein CBLB has not been knocked out. In some alternatives, the NK inhibitory molecule is NKG2A. In some alternatives, the NKG2A expression has been knocked out. In some alternatives, the NKG2A expression has been knocked out by CRISPR/CAS9 system, a zinc finger nuclease or TALEN nuclease. In some alternatives, the NKG2A expression has been knocked out by a CRISPR-related technique. In some alternatives, the knockout of NKG2A expression results in NK cells with higher cytotoxicity against tumor cells than NK cells wherein NKG2A has not been knocked out. In some alternatives, the tumor cells are multiple myeloma cells. In some alternatives, the tumor cells are RPMI8226 cells. In some alternatives, the tumor cells are U266 cells. In some alternatives, the tumor cells are ARH77 cells. In some alternatives, the knockout of NKG2A expression results in NK cells with higher degranulation when stimulated with ICAM-1 and MICA in the presence of an NKG2A agonist antibody than NK cells wherein NKG2A has not been knocked out. In some alternatives, the degranulation is measured by an increase in CD107a. In some alternatives, the knockout of NKG2A expression results in NK cells with a change in the secretion of one or more of GM-CSF, soluble CD137 (sCD137), IFNγ, MIP1α, MIP1β, TNFα or perform, compared to NK cells wherein NKG2A has not been knocked out. In some alternatives, the NK inhibitory molecule is TGFBR2. In some alternatives, the TGFBR2 expression has been knocked out. In some alternatives, the TGFBR2 expression has been knocked out by CRISPR/CAS9 system, a zinc finger nuclease or TALEN nuclease. In some alternatives, the TGFBR2 expression has been knocked out by a CRISPR-related technique. In some alternatives, the knockout of TGFBR2 expression results in resistance to TGFβ mediated inhibition of NK cell cytotoxicity against tumor cells compared to NK cells wherein TGFBR2 has not been knocked out. In some alternatives, the tumor cells are multiple myeloma cells. In some alternatives, the tumor cells are RPMI8226 cells. In some alternatives, the tumor cells are acute myeloid leukemia cells. In some alternatives, the tumor cells are K562 cells. In some alternatives, the tumor cells are chronic myeloid leukemia cells. In some alternatives, the tumor cells are HL-60 cells. In some alternatives, the NK cells are placenta derived (PNK cells). In some alternatives, the natural killer (NK) cells are genetically modified to comprise a modified CD16 is provided. In some alternatives, the modified CD16 has a higher affinity for IgG than wildtype CD16. In some alternatives, the modified CD16 has a valine at position 158 of CD16a. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 is resistant to ADAM17 cleavage. In some alternatives, the CD16 has a proline at position 197 of CD16a. In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the natural killer cells are CD56⁺CD3−CD117⁺CD11a⁺, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives of the method, said natural killer cells additionally express T-bet, GZMB, NKp46, NKp30, and NKG2D. In some alternatives, said natural killer cells express CD94. In some alternatives, said natural killer cells do not express CD94. In some alternatives, the NK cells are placenta derived (PNK cells). In some alternatives of the method, the contacting takes place in vitro. In some alternatives of the method, said contacting takes place in vivo. In some alternatives of the method, said contacting takes place in a human individual. In some alternatives of the method, said method comprises administering said natural killer cells to said individual. In some alternatives of the method, said tumor cells are multiple myeloma cells. In some alternatives of the method, said tumor cells are acute myeloid leukemia (AML) cells. In some alternatives of the method, said individual has relapsed/refractory AML. In some alternatives of the method, said individual has AML that has failed at least one non-innate lymphoid cell (ILC) therapeutic against AML. In some alternatives of the method, said individual is 65 years old or greater, and is in first remission. In some alternatives of the method, said individual has been conditioned with fludarabine, cytarabine, or both, prior to administering said natural killer cells. In some alternatives of the method, said tumor cells are breast cancer cells, head and neck cancer cells, or sarcoma cells. In some alternatives of the method, said tumor cells are primary ductal carcinoma cells, leukemia cells, acute T cell leukemia cells, chronic myeloid lymphoma (CML) cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, or retinoblastoma cells. In some alternatives of the method, said tumor cells are solid tumor cells. In some alternatives of the method, said tumor cells are liver tumor cells. In some alternatives of the method, said tumor cells are lung tumor cells. In some alternatives of the method, said tumor cells are pancreatic tumor cells. In some alternatives of the method, said tumor cells are renal tumor cells. In some alternatives of the method, said tumor cells are glioblastoma multiforme (GBM) cells. In some alternatives of the method, said natural killer cells are administered with an anti-CD33 antibody. In some alternatives, said natural killer cells are administered with an anti-CD20 antibody. In some alternatives, said natural killer cells are administered with an anti-CD138 antibody. In some alternatives, said natural killer cells are administered with an anti-CDF38 antibody. In some alternatives of the method, said natural killer cells have been cryopreserved prior to said contacting or said administering. In some alternatives, said natural killer cells have not been cryopreserved prior to said contacting or said administering. In some alternatives, the natural killer cells are CD56⁺CD3−CD117⁺CD11a⁺, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives of the method, said natural killer cells additionally express T-bet, GZMB, NKp46, NKp30, and NKG2D. In some alternatives, said natural killer cells express CD94. In some alternatives, said natural killer cells do not express CD94.

In some alternatives, a population of natural killer cells is provided, wherein the natural killer (NK) cells are genetically modified to lack expression of an NK inhibitory molecule or manifest a reduced expression of an NK inhibitory molecule. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out. In some alternatives, the NK inhibitory molecule is TGFBR2. In some alternatives, the knockout of TGFBR2 expression generates a population of NK cells having a resistance to TGFβ mediated inhibition of NK cell cytotoxicity against tumor cells compared to NK cells in which TGFBR2 has not been knocked out. In some alternatives, the natural killer (NK) cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the modified CD16 has a proline at position 197 of CD16a. In certain alternatives, the modified CD16 has an amino acid sequence set forth in SEQ ID NO: (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the NK cells are placenta derived (PNK cells). In some alternatives, the natural killer cells are CD56+CD3−CD117+CD11a+, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives, said natural killer cells additionally express T-bet, GZMB, NKp46, NKp30, and/or NKG2D. In some alternatives, said natural killer cells express CD94. In some alternatives, said natural killer cells do not express CD94.

In some alternatives, a method of suppressing the proliferation of tumor cells comprising contacting the tumor cells with natural killer cells from the population of any one of the alternative population of natural killer cells herein are provided. In some alternatives, the population of natural killer cells is provided, wherein the natural killer (NK) cells are genetically modified to lack expression of an NK inhibitory molecule or manifest a reduced expression of an NK inhibitory molecule. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out. In some alternatives, the NK inhibitory molecule is TGFBR2. In some alternatives, the knockout of TGFBR2 expression generates a population of NK cells having a resistance to TGFβ mediated inhibition of NK cell cytotoxicity against tumor cells compared to NK cells in which TGFBR2 has not been knocked out. In some alternatives, the natural killer (NK) cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the modified CD16 has a proline at position 197 of CD16a. In certain alternatives, the modified CD16 has an amino acid sequence set forth in SEQ ID NO: (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 contains an IgK signal peptide. In some alternatives, the modified CD16 contains a CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the modified CD16 is introduced via a retroviral vector. In some alternatives, the retroviral vector comprises one or more drug selection markers. In some alternatives, the NK cells are placenta derived (PNK cells). In some alternatives, the natural killer cells are CD56+CD3-CD117+CD11a+, express perform and/or EOMES, and do not express one or more of RORγt, aryl hydrocarbon receptor, and IL1R1. In some alternatives, said natural killer cells express perform and EOMES, and do not express any of RORγt, aryl hydrocarbon receptor, or IL1R1. In some alternatives, said natural killer cells additionally express T-bet, GZMB, NKp46, NKp30, and/or NKG2D. In some alternatives, said natural killer cells express CD94. In some alternatives, said natural killer cells do not express CD94. In some alternatives, the population of natural killer cells derived from placenta or parts thereof, thereby comprising placenta derived NK cells (pNK cells), wherein the pNK cells are genetically modified such that they lack expression of an NK inhibitory molecule or manifest reduced expression of an NK inhibitory molecule, are provided. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives, the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and/or perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives, the increase in CD107a is measured by FACs. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out, such as naturally occurring NK cells. In some alternatives, the population of cells are of placental derived natural killer cells (pNK), wherein the pNK cells are genetically modified to comprise a modified CD16. In some alternatives, the modified CD16 has a higher affinity for IgG than wildtype CD16. In some alternatives, the modified CD16 has a valine at position 158 of CD16a. In some alternatives, the modified CD16 is resistant to ADAM17 cleavage. In some alternatives, the CD16 has a proline at position 197 of CD16a. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 contains an IgK signal peptide or CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the selection marker include genes encoding a protein conferring resistance to a selection agent such as PuroR gene, ZeoR gene, HygroR gene, neoR gene, and/or the blasticidin resistance gene. In some alternatives, the modified CD16 is introduced via a retroviral vector. In some alternatives, the retroviral vector comprises one or more drug selection markers. In some alternatives of the method, said contacting takes place in vitro. In some alternatives of the method, said contacting takes place in vivo. In some alternatives of the method, said contacting takes place in a human individual, preferably an individual selected to receive an anticancer therapy. In some alternatives of the method, said method comprises administering said natural killer cells to said individual. In some alternatives of the method, said tumor cells are multiple myeloma cells. In some alternatives of the method, said tumor cells are acute myeloid leukemia (AML) cells. In some alternatives of the method, said individual has relapsed/refractory AML. In some alternatives of the method, said individual has AML that has failed at least one non-innate lymphoid cell (ILC) therapeutic against AML. In some alternatives of the method, said individual is 65 years old or greater, and is in first remission. In some alternatives of the method, said individual has been conditioned with fludarabine, cytarabine, or both, prior to administering said natural killer cells. In some alternatives of the method, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives of the method, the tumor cells are solid tumor cells. In some alternatives of the method, the solid tumor cells are selected from the group consisting of liver tumor cells, lung tumor cells, pancreatic tumor cells, renal tumor cells, and glioblastoma multiforme (GBM) cells. In some alternatives of the method said natural killer cells are administered with an anti-CD33 antibody. In some alternatives of the method, said natural killer cells are administered with an anti-CD20 antibody. In some alternatives of the method, said natural killer cells are administered with an anti-CD138 antibody. In some alternatives of the method, said natural killer cells are administered with an anti-CD38 antibody. In some alternatives of the method, said natural killer cells have been cryopreserved prior to said contacting or said administering. In some alternatives of the method, said natural killer cells have not been cryopreserved prior to said contacting or said administering.

In a third aspect, a population of natural killer cells derived from placenta or parts thereof, thereby comprising placenta derived NK cells (pNK cells), wherein the pNK cells are genetically modified such that they lack expression of an NK inhibitory molecule or manifest reduced expression of an NK inhibitory molecule, are provided. In some alternatives, the NK inhibitory molecule is one or more NK inhibitory molecules selected from the group consisting of CBLB, NKG2A and TGFBR2. In some alternatives, the genetically modified NK cells have a higher cytotoxicity against tumor cells than NK cells in which expression of the NK inhibitory molecule has not been knocked out or reduced. In some alternatives, the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, and retinoblastoma cells. In some alternatives, the tumor cells are solid tumor cells. In some alternatives, the solid tumor cells are selected from the group consisting of liver tumor cells, lung tumor cells, pancreatic tumor cells, renal tumor cells, and glioblastoma multiforme (GBM) cells. In some alternatives, expression of the NK inhibitory molecule has been knocked out. In some alternatives, expression of the NK inhibitory molecule has been knocked out by CRISPR/CAS9 system, a zinc finger nuclease or TALEN nuclease. In some alternatives, expression of the NK inhibitory molecule has been knocked out by a CRISPR-related technique. In some alternatives, the NK inhibitory molecule is CBLB. In some alternatives the knockout of CBLB expression generates a population of NK cells having a higher IFNγ secretion when stimulated with ICAM-1 and MICA than NK cells in which CBLB has not been knocked out. In some alternatives, the knockout of CBLB expression generates a population of NK cells having a higher degranulation when stimulated with ICAM-1 and MICA than NK cells in which CBLB has not been knocked out. In some alternatives, the degranulation is measured by an increase in CD107a. In some alternatives, the knockout of CBLB expression generates a population of NK cells having a change in the secretion of one or more of GM-CSF, soluble CD137 (sCD137), IFNγ, MIP1α, MIP1β, TNFα and/or perform when co-cultured with multiple myeloma cells, compared to NK cells in which CBLB has not been knocked out. In some alternatives, the NK inhibitory molecule is NKG2A. In some alternatives, the knockout of NKG2A expression generates a population of NK cells having a higher degranulation when stimulated with ICAM-1 and MICA in the presence of an NKG2A agonist antibody than NK cells in which NKG2A has not been knocked out. In some alternatives, the degranulation is measured by an increase in CD107a. In some alternatives the increase in CD107a is measured by FACs. In some alternatives, the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out, such as naturally occurring NK cells.

In a fourth aspect, a population of placental derived natural killer cells (pNK), wherein the pNK cells are genetically modified to comprise a modified CD16. In some alternatives the modified CD16 has a higher affinity for IgG than wildtype CD16. In some alternatives the modified CD16 has a valine at position 158 of CD16a. In some alternatives, the modified CD16 is resistant to ADAM17 cleavage. In some alternatives, the CD16 has a proline at position 197 of CD16a. In certain alternatives, the modified CD16 has an amino acid sequence set forth in SEQ ID NO: 1 (MWQLLLPTALLLLVSAGMRTEDLPKAVVFLEPQWYRVLEKDSVTLKCQGAYSPED NSTQWFHNESLISSQASSYFIDAATVDDSGEYRCQTNLSTLSDPVQLEVHIGWLLLQ APRWVFKEEDPIHLRCHSWKNTALHKVTYLQNGKGRKYFHHNSDFYIPKATLKDSG SYFCRGLVGSKNVSSETVNITITQGLAVPTISSFFPPGYQVSFCLVMVLLFAVDTGLYF SVKTNIRSSTRDWKDHKFKWRKDPQDK; SEQ ID NO: 1). In some alternatives, the modified CD16 contains an IgK signal peptide or CD16 signal peptide. In some alternatives, the modified CD16 is introduced into the NK cells via viral infection. In some alternatives, the modified CD16 is introduced into hematopoietic cells via viral infection, which hematopoietic cells are then differentiated into NK cells. In some alternatives, the modified CD16 is introduced via a lentiviral vector. In some alternatives, the lentiviral vector has either a CMV or an EF1α promoter. In some alternatives, the lentiviral vector comprises one or more drug selection markers. In some alternatives, the selection marker include genes encoding a protein conferring resistance to a selection agent such as PuroR gene, ZeoR gene, HygroR gene, neoR gene, and/or the blasticidin resistance gene. In some alternatives, the modified CD16 is introduced via a retroviral vector. In some alternatives, the retroviral vector comprises one or more drug selection markers.

a. 7.1 Alternative 1: CBLB Knockout Three-Stage NK Cells

i. 7.1.1 CBLB Knockout Characterization

CBLB knockout NK cells were generated by performing a CRISPR knockout of the CBLB gene in NK cells during day 3, 5, or 7 of the 35-day, three-stage process for producing NK cells, as described herein and in International Patent Application Publication No. WO 2016/109661, which is incorporated by reference herein in its entirety.

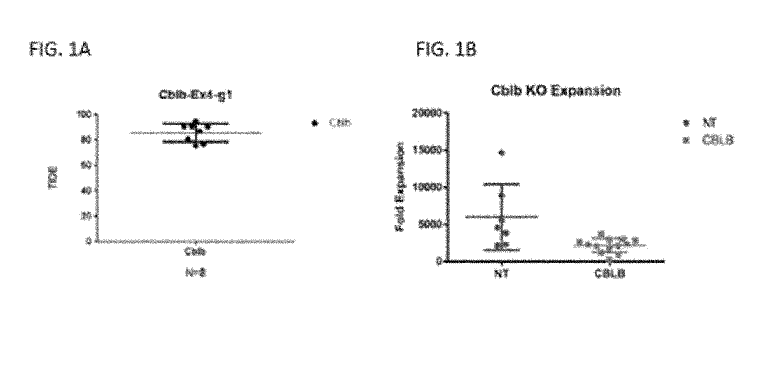

The average efficiency of the CBLB knockout is above 80% at day 35 of the 35 day process as measured by the TIDE (Tracking of Indels by DEcomposition) assay (FIG. 1A).

Fold expansion of the NK cells post-knockout was measured, and the percentage of live cells and CD3⁻CD56⁺ cells were determined. Fold expansion was reduced compared to untreated cells (FIG. 1B), but the proportion of live cells and CD3⁻CD56⁺ cells was comparable to untreated cells in CBLB knock out NK cells.

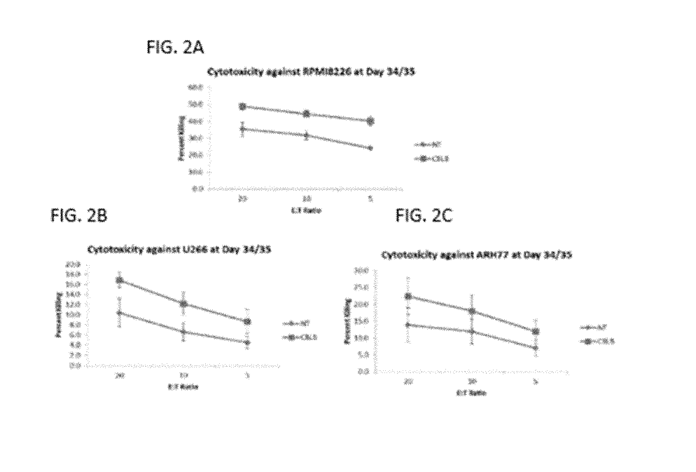

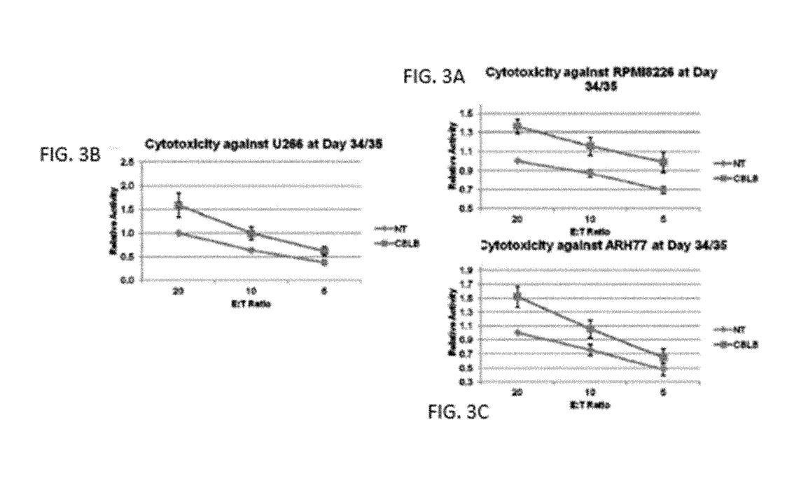

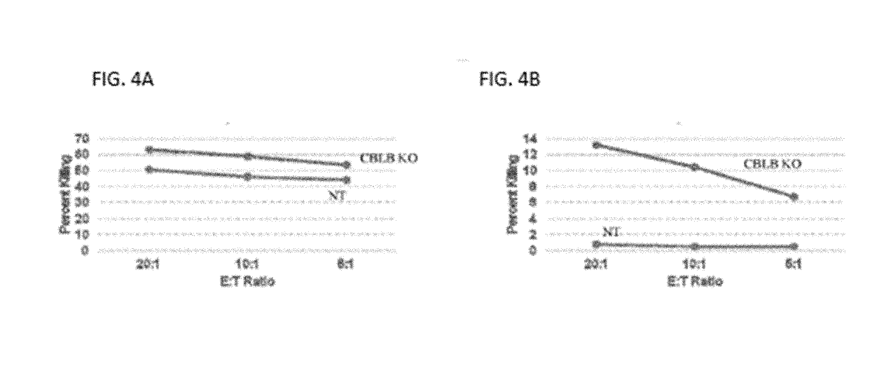

At day 34 or 35 of the three-stage process, cytotoxicity against various multiple myeloma cell lines (RPMI8226, U266, ARH277) was determined at effector:target (E:T) ratios of 20:1, 10:1, and 5:1 (FIG. 2A-C). The CBLB knockout NK cells were shown to have increased cytotoxicity in comparison with untreated cells for each of the cell lines tested and at all ratios (FIG. 3A-C). The cytotoxicity data was then normalized, and the CBLB knockout NK cells were shown to have up to a four-fold increase in cytotoxicity in comparison to untreated cells. Cytotoxicity of CBLB knockout NK cells against HL60 and KG1 cells was also determined, as shown in FIG. 4A-B.

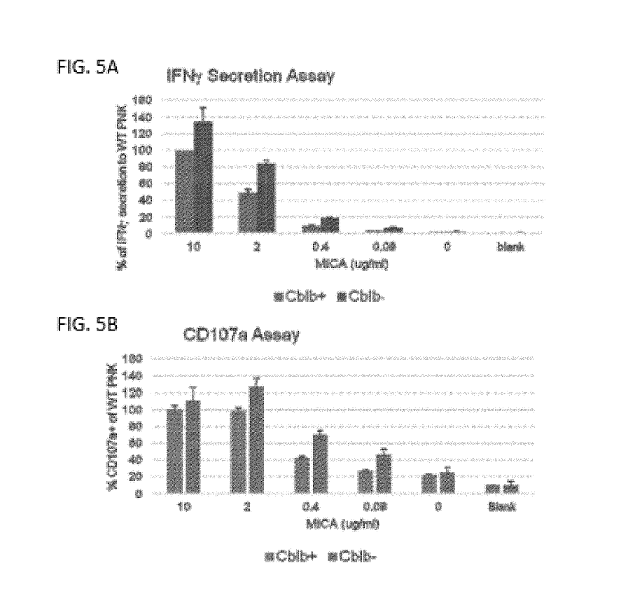

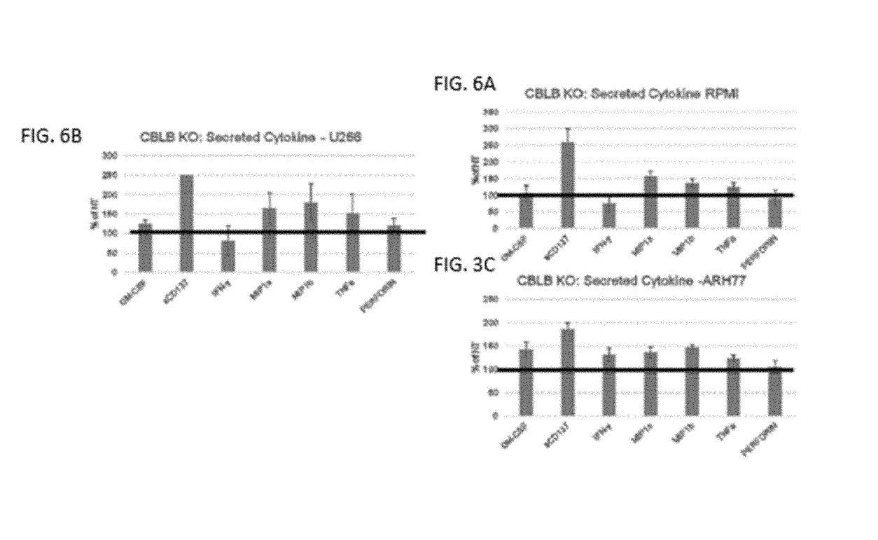

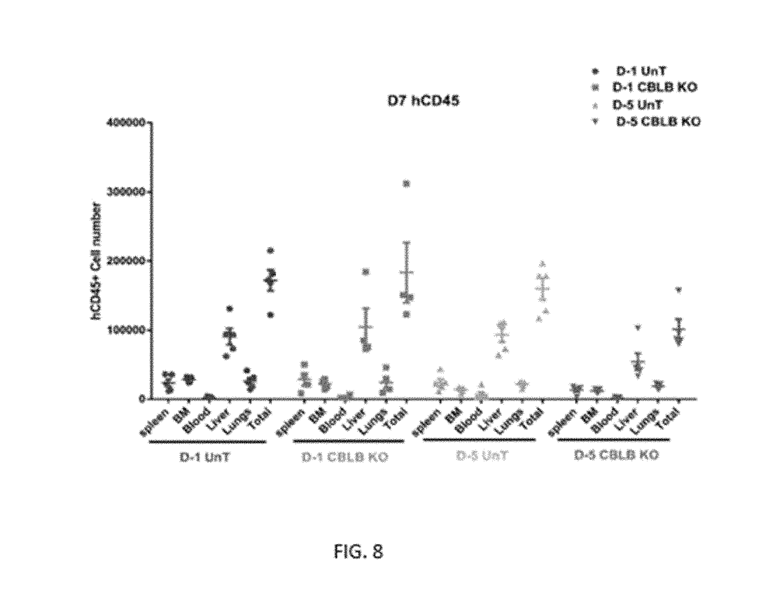

In addition to cytotoxicity data, the level of IFNγ secretion and CD107a, a measure of degranulation, upon stimulation with MHC Class I polypeptide related sequence A (MICA) and ICAM-1, was measured. The IFNγ secretion levels of CBLB knockout and untreated NK cells with varying levels of MICA stimulation in the presence of a consistent level of ICAM-1 are shown in FIG. 5A. The results of the CD107a assay in both CBLB knockout and untreated NK cells with varying levels of MICA stimulation in the presence of a consistent level of ICAM-1 are shown in FIG. 5B. As shown in 5A and 5B untreated is the bar graph to the right and treated is the bar graph to the left, in the pairs of bar graphs in the FIG. 8

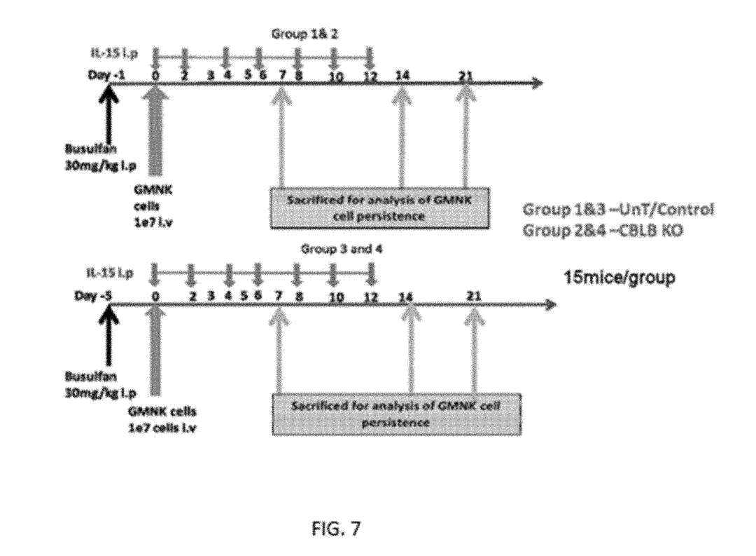

Cytokine secretion of GM-CSF, sCD137, IFNγ, MIP1α, MIP1β, TNFα, and perform were also measured in the presence of multiple myeloma cells lines RPMI, U266, or ARH77, without MICA stimulation. The results of the cytokine secretion assay are shown in FIG. 6A-C.

ii. 7.1.2. CBLB Knockout Pre-Clinical Data

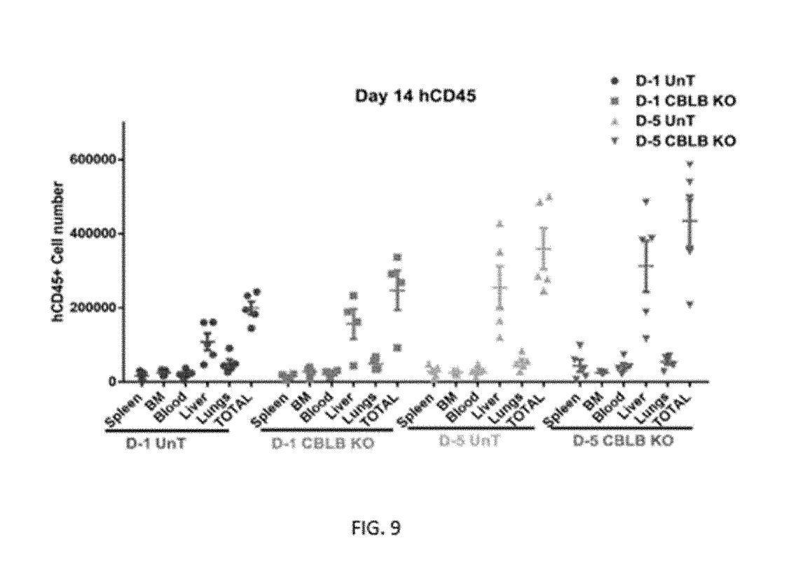

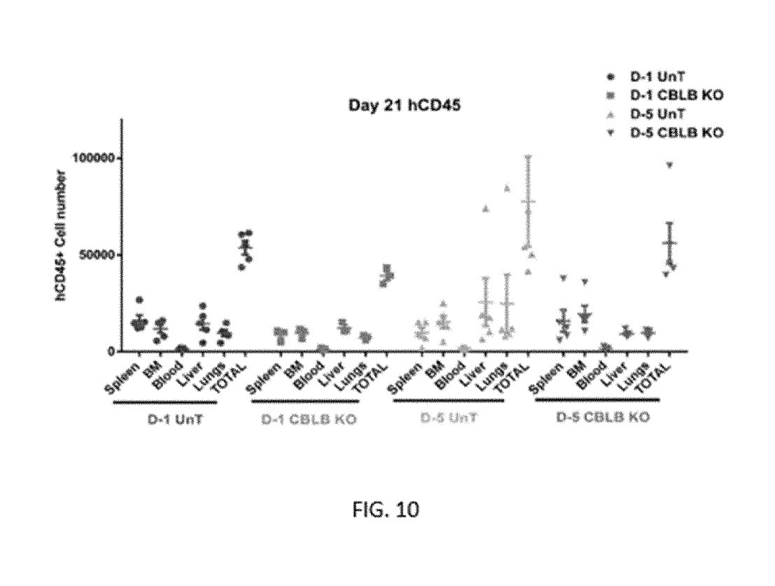

To determine the biodistribution and persistence of CBLB knockout NK cells in vivo, as produced in Example 7.1.1, a study was designed as shown in FIG. 7. Two groups, one with busulfan (an anti-neoplastic agent) precondition at day-1, and one with busulfan preconditioning at day-5, were studied. Seven, fourteen, and twenty-one days after cell infusion into NOD SCID gamma (NSG) mouse tissues, human CD45⁺ counts were taken in the spleen, bone marrow (BM), blood, liver, and lungs, and total counts were also tallied (FIGS. 8-10). Biodistribution and persistence was found to be similar in untreated and CBLB knockout NK cells after seven days (FIG. 8). After fourteen days, the similarity in biodistribution and persistence was maintained between groups, although the absolute number of human CD45⁺ counts were higher. (FIG. 8-9). After twenty-one days, persistence and biodistribution continued to be similar, but the absolute number of human CD45⁺ counts dropped, suggesting the persistence was associated with IL-15 supplementation (FIG. 10). IL-15 is a cytokine, which induces cell proliferation of NK cells.

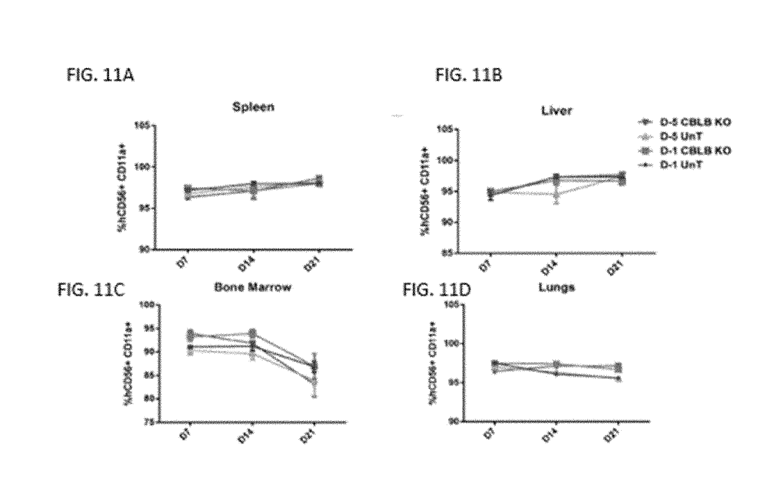

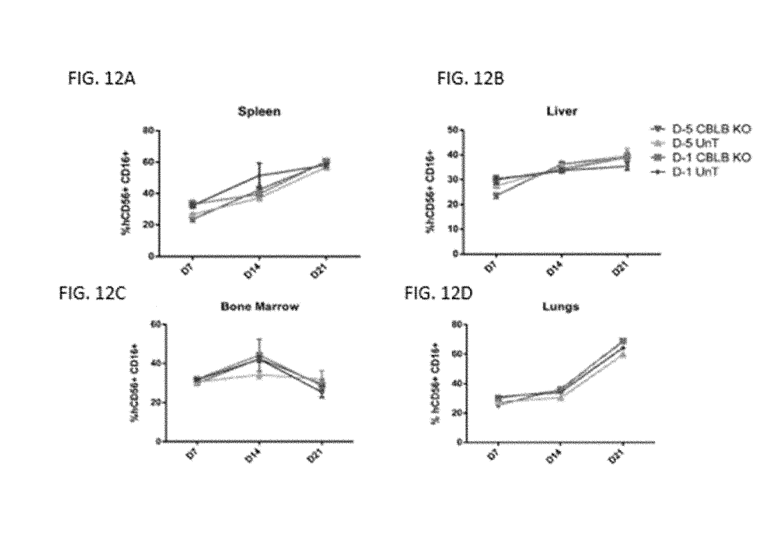

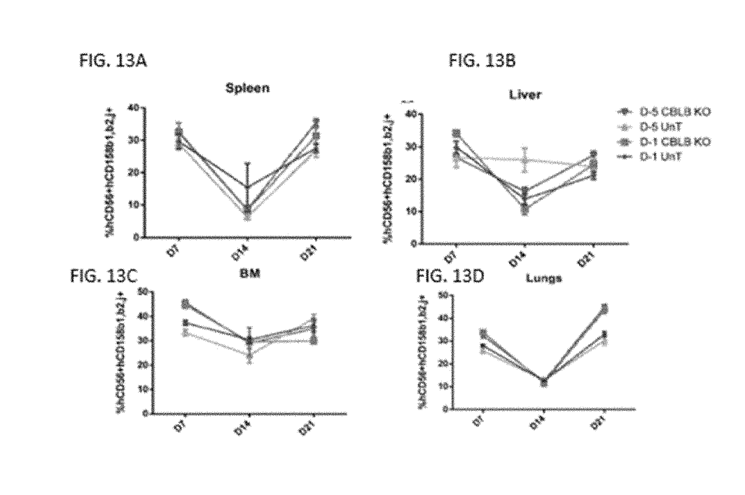

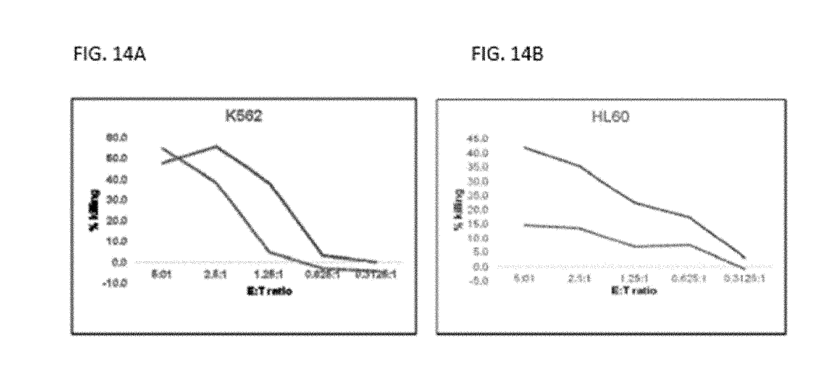

On days seven, fourteen, and twenty-one, the presence of CD56⁺CD11a⁺ cells were also measured in spleen, liver, bone marrow, and lungs (FIG. 11). Similar frequencies of CD56⁺CD11a⁺ cells were found for the untreated and CBLB knockout conditions, and lower frequencies of CD56⁺CD11a⁺ cells were found in bone marrow compared to the other tissues (FIG. 11). CD16 and KIR expression in spleen, liver, bone marrow, and lungs was also measured, and similar frequencies found for the untreated and CBLB knockout conditions (FIGS. 12 and 13). It was noted that CD16 and KIR expression both increased in vivo in comparison with the pre-infusion profile.

Proliferation of NK cells in NSG mice was measured, and CBLB knockout NK cells were shown to proliferate more rapidly than control treated cells by day 14 of NK cell administration.

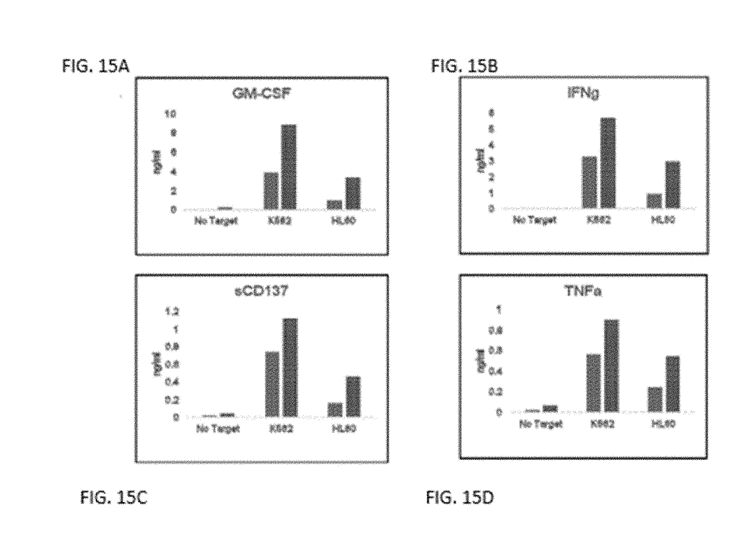

NK cells isolated from NSG mouse tissues 14 days after administration were purified, and cytotoxicity against K562 and HL60 cells lines was determined (FIG. 14A-B). CBLB knockout NK cells were shown to have enhanced cytotoxicity in comparison with control treated cells against both cells lines ex vivo (FIG. 14A-B). In FIG. 14A to 14B, the control is shown as the lower percent killer in both graphs. The ex vivo isolated CBLB knockout cells were also shown to release increased levels of GM-CSF, IFNγ, sCD137, and TNFα cytokines in tumor cell co-cultures, in comparison with control treated cells (FIG. 15A-D). Thus, CBLB knockout NK cells retain their enhanced functional activity after fourteen days in NSG mice.

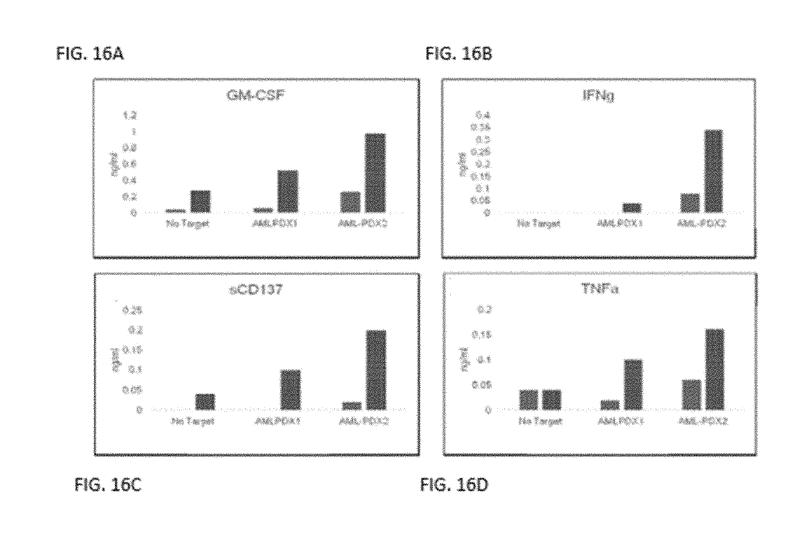

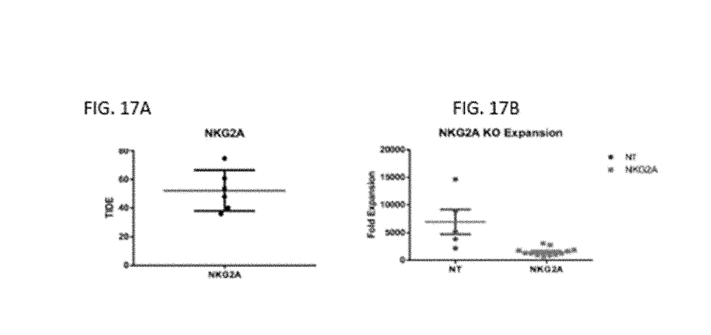

Finally, functional activity of CBLB knockout NK cells were tested against freshly isolated patient derived AML xenografts (PDX) (FIG. 16A-D). The CBLB knockout NK cells exhibited increased secreted GM-CSF, IFNγ, sCD137, and TNFα compared with control (FIG. 16A-D).

b. 7.2 Alternative 2: NKG2A Knockout Three-Stage NK Cells

NKG2A knockout NK cells were generated by performing a CRISPR knockout of the NKG2A gene in NK cells during day 3, 5, or 7 of the 35-day, three-stage process for producing NK cells, as described herein and in International Patent Application Publication No. WO 2016/109661, which is incorporated by reference herein in its entirety.

The average efficiency of the NKG2A knockout is about 60% at day 35 of the 35 day process as measured by the TIDE (Tracking of Indels by DEcomposition) assay (FIG. 17A).

Fold expansion of the NK cells post-knockout was measured, and the percentage of live cells and CD3⁻CD56⁺ cells are determined. Fold expansion was reduced compared to untreated cells (FIG. 17B), but the proportion of live cells and CD3⁻CD56⁺ cells was comparable to untreated cells in NKG2A knockout NK cells.

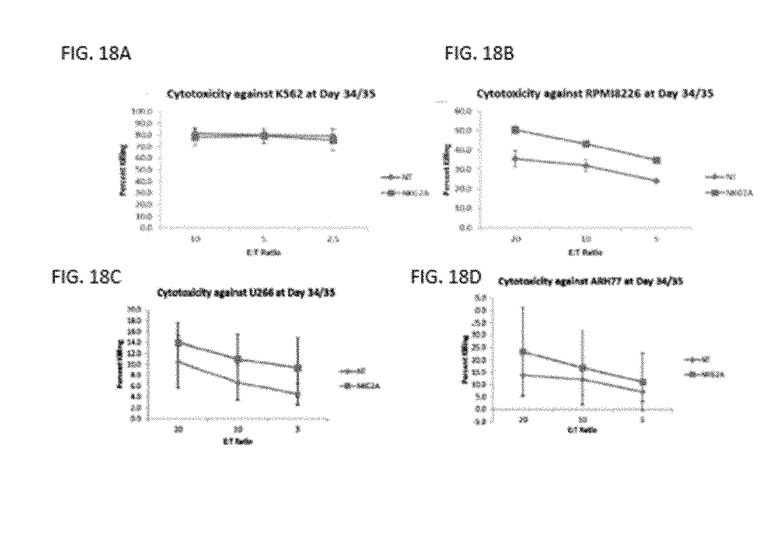

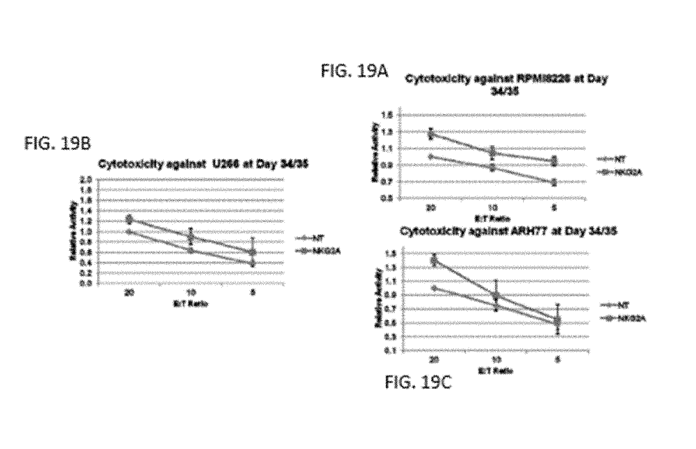

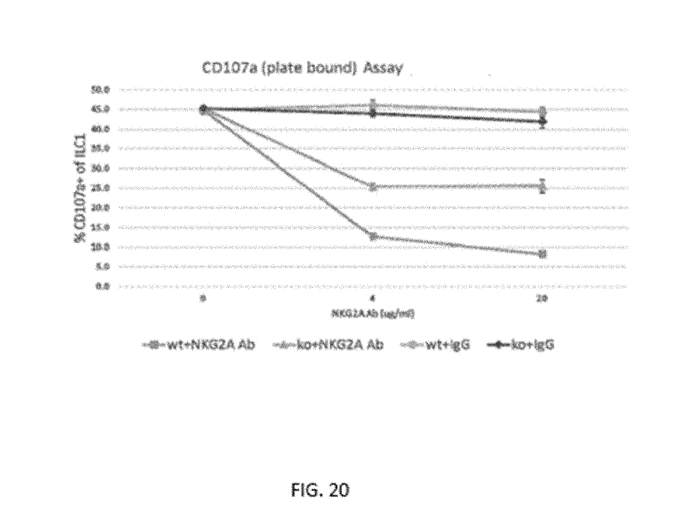

At day 34 or 35 of the three-stage process, cytotoxicity against various multiple myeloma cell lines (RPMI8226, U266, ARH277) was determined at E:T ratios of 20:1, 10:1, and 5:1 (FIG. 18A-D). Cytotoxicity against K562 cells was determined at E:T ratios of 10:1, 5:1, and 2.5:1. The NKG2A knockout NK cells were shown to have increased cytotoxicity in comparison with untreated cells for each of the RPMI8226, U266, and ARH277 cell lines and at all ratios (FIG. 19A-C), but had comparable cytotoxicity with untreated cells against K562 cells. It is hypothesized that cytotoxicity against K562 cells had reached maximum levels. The cytotoxicity data against the multiple myeloma cell lines was then normalized, and the NKG2A knockout NK cells were shown to have up to a three-fold increase in cytotoxicity in comparison to untreated cells. In some alternatives herein, NKG2A knockout NK cells were shown to have up to a three-fold increase in cytotoxicity in comparison to untreated cells.

A plate bound degranulation assay was performed to test the response of NKG2A knockout NK cells to an NKG2A agonist antibody in the presence of MICA and ICAM-1 stimulation (FIG. 20). In the presence of a control IgG antibody, the NKG2A knockout cells showed high activity (percent CD107a), just like control NK cells with wild type NKG2A. Control (non-knockout) NK cells in the presence of the NKG2A agonist antibody showed low activity as expected. The NKG2A knockout NK cells in the presence of the NKG2A agonist antibody showed an intermediate activity. Thus, the NKG2A agonist antibody was found to reduce control NK cell activity, but was less effective in the NKG2A knockout cells, indicating these cells are more resistant to NKG2A mediated inhibitory signal.

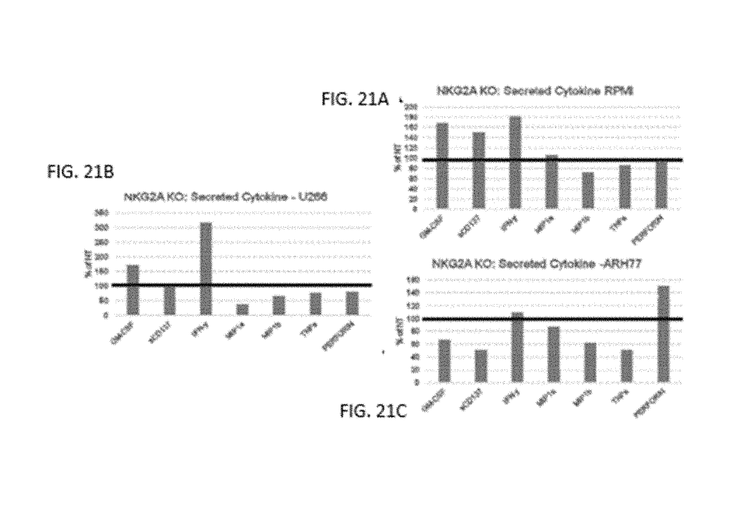

Cytokine secretion of GM-CSF, sCD137, IFNγ, MIP1α, MIP1β, TNFα, and perform were also measured in the presence of multiple myeloma cells lines RPMI, U266, and ARH77, without MICA stimulation. The results of the cytokine secretion assay are shown in FIG. 21A-C.

c. 7.3 Alternative 3: TGF-β Knockout Three-Stage NK Cells

Using a CRISPR-related technique, TGF-β receptor II (TGFBR2) was knocked out of in NK-92 cells, resulting in a significant decrease in TGFBR2 expression. Results were further validated in NK-92 cells by showing that phosphorylated Smad2/3 (pSmad2/3) was reduced, indicating blockade of the TGF-β signaling pathway. TGF-β triggered activating marker (NKp30) down-regulation was also abolished in these cells.

TGFBR2 knockout NK cells were then generated by performing a CRISPR knockout of the TGFBR2 gene in NK cells during day 0, 5, 10, or 14 of the 35-day, three-stage process for producing NK cells, as described herein and in International Patent Application Publication No. WO 2016/109661, which is incorporated by reference herein in its entirety. Characterization of the day 5 knockout is described below.

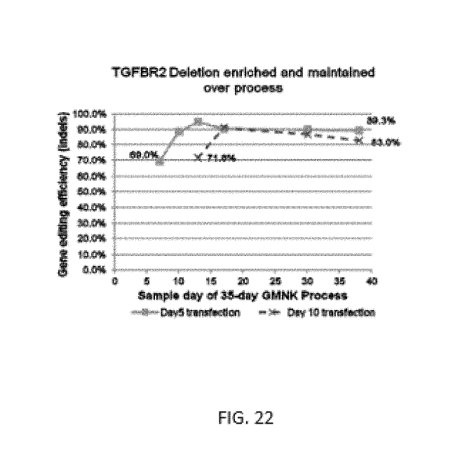

The efficiency of the TGFBR2 knockout enriched quickly from 70% at day 5 to above 80%, and remained stable throughout the 35 day process (FIG. 22). Likewise, the mutation spectrum stayed unchanged throughout the process. As with NK-92 cells, the day 5 TGF-β knockout GM NK cells showed a blockade of TGF-β signaling, resulting in reduced pSmad2-3. Activating receptor down-regulation was also abrogated in the TGFBR2 knockout cells, for receptors such as DNAM-1, NKG2D and NKp30. Differentiation of the TGFBR2 knockout NK cells was found to be similar to the control, untreated group, as shown in Table 1.

TABLE 1
Immunophenotyping by flow cytometry of TGFBR2 KO vs.
control NK cells.

| | Live Cells | CD3⁻CD56⁺ | CD16⁺ | ILC1 | ILC3 |
|---|---|---|---|---|---|
| | (%) | (%) | (%) | (%) | (%) |
| TGFBR2 KO | 89.2 | 93.4 | 19 | 78.4 | 21.6 |
| Control | 90.7 | 95.9 | 24.2 | 87.3 | 12.7 |

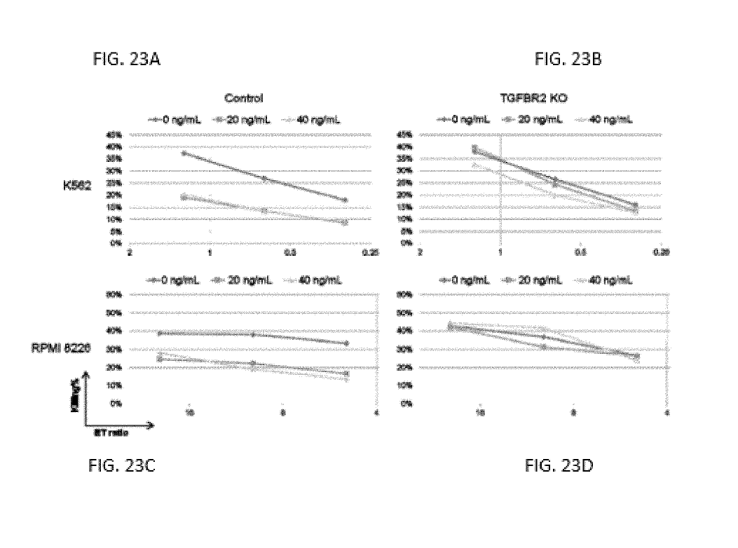

At day 34 or 35, cytotoxicity against K562 and RPMI8226 cell lines was determined at a range of E:T ratios. Cytotoxicity was similar in the TGFBR2 knockout NK cells to the cytotoxicity in the control without TGF-β1 treatment (FIG. 23A-D), and TGFBR2 knockout NK cells were shown to impart resistance to TGF-β1 inhibition during the cytotoxicity assay.

Genetic and phenotypic analyses of the results of TGFBR2 knockout at different days of transfection and in different cell batches are shown in Table 2. High levels of TGFBR2 deletion (88.1% average) were achieved for GM NK across multiple donors at different time points. These results were confirmed by corresponding phenotypic changes, such as signaling blockade and changes in NK marker downregulation.

TABLE 2
Genetic and phenotypic analyses of the results of TGFBR2
knockout at different days of transfection and in different cell batches.

| | | Gene KO | | |
|---|---|---|---|---|
| Transfection | | efficiency | Smad2/3 | activating marker |
| Day | Batch | (indel %) | phosphorylation | down regulation |
| 14 | A | 96% | blocked | blocked |
| | B | 91% | blocked | blocked |
| 10 | C | 83% | blocked | blocked |
| 5 | C | 89% | blocked | blocked |
| | D | 95% | blocked | blocked |
| | E | 93% | blocked | blocked |
| | F | 73% | Not tested | Not tested |
| 0 | G | 92% | Not tested | Not tested |
| | H | 81% | Not tested | Not tested |

Average KO efficiency = 88.1% (SD 8.6%)

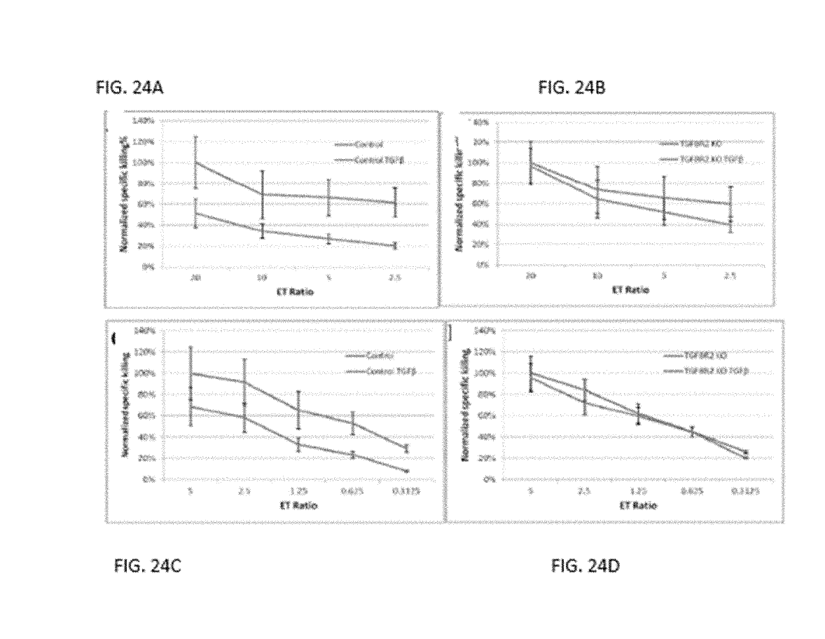

Effector function of TGFBR2 knockout cells was also tested against HL60 cells and K562 cells in a four hour cytotoxicity assay (FIG. 24A-D). TGFBR2 knockout cells demonstrated resistance to the TGFβ1-triggered inhibition on antitumor cytotoxicity in both the HL60 and K562 cells (FIG. 24A-D).

d. 7.4 Alternative 4: NK Cells with Modified CD16

Lentiviral vectors comprising genetically modified CD16 were developed, as indicated in Table 3.

TABLE 3
CD16 lentiviral constructs for GM NK cells.

| Signal Peptide/ | | |
|---|---|---|
| Promoter | EC Domain Modification | Name |
| IgK-EF1a | High IgG binding 158V; | kCD16VP |
| | ADAM17 Resistance 197P | |
| CD16-EF1a | High IgG binding 158V; | CD16VP |
| | ADAM17 Resistance 197P | |
| CD16-EF1a | Wild Type | CD16WT |
| CD16-EF1a | ADAM17 Resistance 179P | CD16P |
| CD16-CMV | High IgG binding 158V; | VB-CD16VP |
| | ADAM17 Resistance 197P | |
| CD16-CMV | High IgG binding 158V; | VB-CD16VPO |
| | ADAM17 Resistance 197P | |

Two different signal peptides, IgK and CD16, were used, and two different promoters, EF1α and CMV, were used, along with the desired mutations-the high IgG binding affinity mutant F158V and the ADAM17 resistance mutant S197P. The lentiviral vectors were also designed for puromycin selection for enrichment post-transduction.

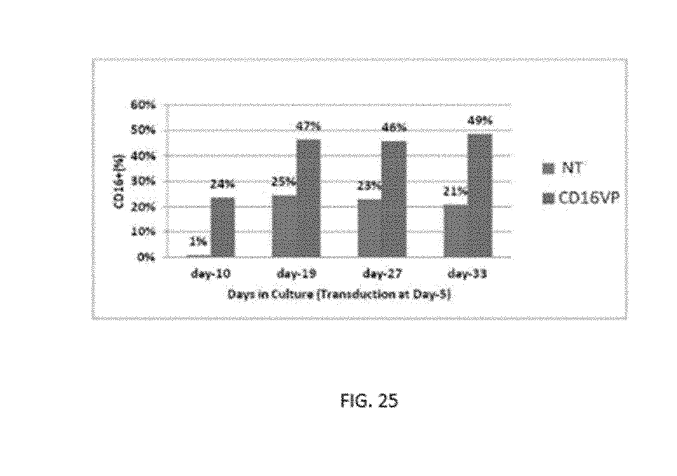

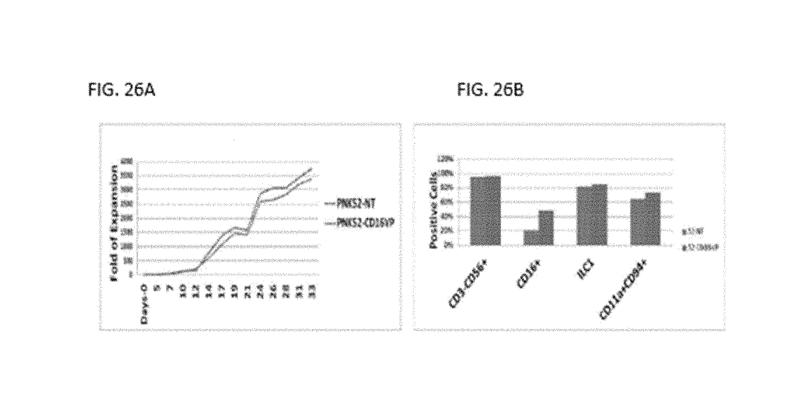

Persistence of CD16 expression in 35-day, three-stage process for producing NK cells, as described herein and in International Patent Application Publication No. WO 2016/109661 (incorporated by reference herein in its entirety), was tested for NK cells transduced on day 5. CD16 expression was determined by FACS using an anti-CD16⁻ FITC antibody (BD Cat#555406, clone 3G8). Stable and higher levels of CD16 were evident in the CD16VP transduced cells compared to wildtype (FIG. 25). (Right bar graph in the pairs of bar graphs in FIG. 25). Thus, the feasibility of using lentiviral vectors to deliver genetically modified CD16 to 35-day, three-stage process NK cells was confirmed.

To determine the amount of CD16 shedding resulting from CD16 cleavage, an assay was developed wherein NK cells were treated with the proteinase inhibitor TAPI at 50 μM for 30 minutes, then with or without PMA (1 m/mL) for 4 hours. PMA activation was shown to reduce CD16 in peripheral blood NK cells by 97% and in 35-day, three-stage process NK cells by 89%. TAPI treatment was able to inhibit CD16 shedding in both peripheral blood and three-stage NK cells. NK cells transduced with CD16VP showed resistance to PMA induced CD16 shedding. In non-treated cells, 94% of CD16 was shed, whereas only 17% of CD16 was shed in CD16VP transduced cells.

The proliferation and phenotype of 35-day, three-stage process NK cells transduced with CD16VP was compared to untreated cells. No significant difference in proliferation or in NK maturation markers was found between the transduced and untreated cells (FIG. 26A-B). (In FIG. 26B, the left bar graph of the pair of bar graphs represents the untreated. The right bar graph in the pair of bar graphs in FIG. 26 B represents the CD16VP transduced cells.

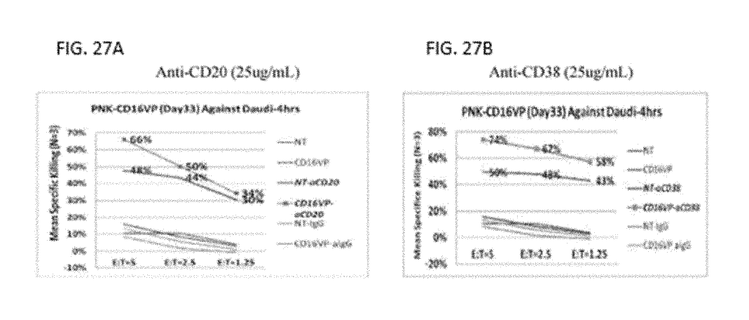

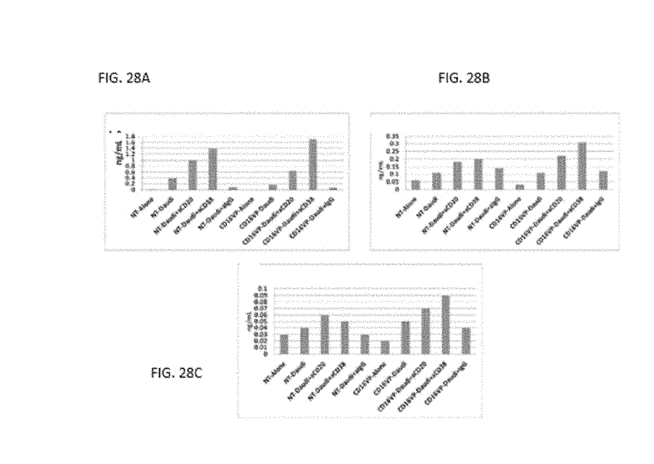

Antibody-dependent cell-mediated cytotoxicity (ADCC) was studied to assess the effects of the transduction with CD16VP. Target cancer cells (Daudi or U266) were incubated with mAb (anti-CD20 or anti-CD38) for 30 minutes, with no mAb and IgG used as controls. Effector cells and cancer cells were added together at an E:T ratio of 1.25:1, and a control without effector cells was also performed. Topo5 was added to stain for live cells. FACS analysis determined the percentage of specific killing by effector cells. CD16VP transduced cells were found to have improved ADCC against Daudi cells compared to untreated NK cells, with both anti-CD20 and anti-CD38 antibodies (FIG. 27A-B). Secretion of IFN-γ, GM-CSF, and TNF-α was also tested during 24 hour ADCC at an E:T of 1:1, and the CD16VP transduced NK cells showed increased cytokine secretion compared to untreated NK cells (FIG. 28A-C).

e. 7.5 Alternative 5: TGFBR2/CBLB Double Knockout Three-Stage NK Cells

Using a CRISPR-related technique, a knockout of TGFBR2 and CBLB genes was performed on Day 5 GM NK cells to create the following populations: mock transfection, TGFBR2 knockout GM NK, CBLB knockout GM NK, and TGFBR2/CBLB double knockout GM NK. Gene editing efficiency was assessed by targeted amplicon sequencing combined with TIDE (Tracking of Indels by DEcomposition) analysis. Immunophenotyping for GM NK cells and controls was carried out following a routine immunophenotyping protocol. Cells were treated with or without TGFβ1 for 48 hours prior to effector function and secreted analyte evaluation. To determine effector function in hematological cancer cell lines, i.e., K562, HL60, KG-1 and RPMI8266, a 4-hour flow-based cytotoxicity assay was utilized. For secreted analyte evaluation, the GM NK and controls were co-cultured with the hematological cancer cell lines for 24 hours at a 1:1 E:T ratio. Supernatant was collected and stored at −20° C. until being analyzed by Luminex Multiplex immunoassay.

Gene Knockout Efficiency for Double Knockout GM NK.

Knock out efficiency was comparable for each TGFBR2 and CBLB locus in the double knockout to the single knockout controls, see Table 4.

TABLE 4
Knock out efficiency of double knockout GM NK.

KO efficiency

| Test Group | TGFBR2 | CBLB |
|---|---|---|
| Cas9 | N.D. | N.D. |
| TGFBR2 GMNK | 73.2% | N.D. |
| CBLB GMNK | N.D. | 75.9% |
| Double KO GMNK | 68% | 76.7% |

N.D. indicates no data/not determined.

Immunophenotype of Double Knockout GM NK.

Immunophenotypic analyses showed that double knockout GM NK had similar phenotype as controls (Table 5).

TABLE 5
Phenotypic analysis of double knock out GM NK.

| | Live Cells | CD3⁻CD56⁺ | CD16⁺ | ILC1 | ILC3 | CD34⁺ |
|---|---|---|---|---|---|---|
| Test Group | (%) | (%) | (%) | (%) | (%) | (%) |
| Cas9 | 93.4 | 89.0 | 34.2 | 73.0 | 26.6 | 1.0 |
| TGFBR2 GMNK | 93.8 | 89.4 | 31.6 | 73.1 | 26.3 | 0.6 |
| CBLB GMNK | 90.6 | 92.6 | 31.1 | 76.5 | 23.2 | 0.7 |
| Double KO GMNK | 86.9 | 88.9 | 25.7 | 71.2 | 28.4 | 0.6 |

Fold Expansion of Double Knockout GM NK.

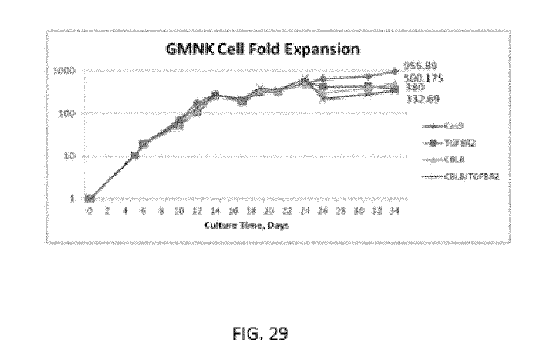

Overall fold expansion showed a trend that single knockouts expanded less than the mock transfection control and the double knock out had a further decrease in expansion than single knockouts (FIG. 29).

Cytotoxicity Assay.

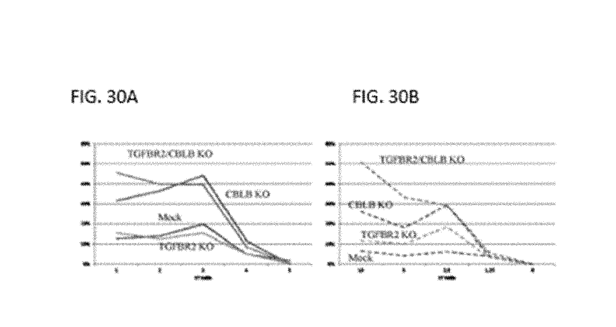

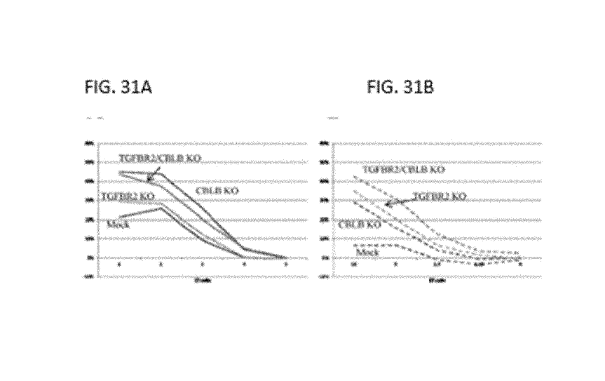

Double knock out GM NK demonstrated the combined benefits from both single knock outs. The double knock out showed both the augmented specific killing of CBLB-GM NK and the insensitivity to TGFβ-triggered inhibition of TGFBR2-GM NK. Thus, in the presence of TGFβ, the double knockout GM NK cells exhibited the most killing against target tumor cell lines, as seen in FIGS. 30 and 31.

Secreted Analytes in Co-Culture Supernatant.

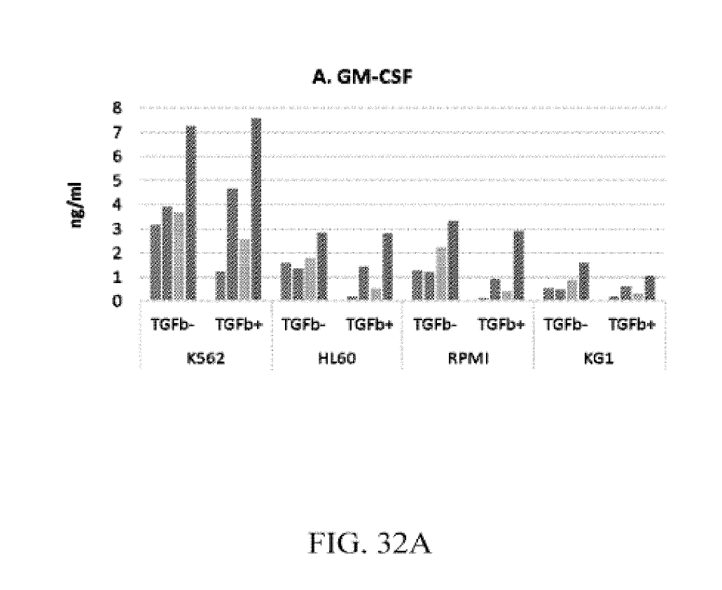

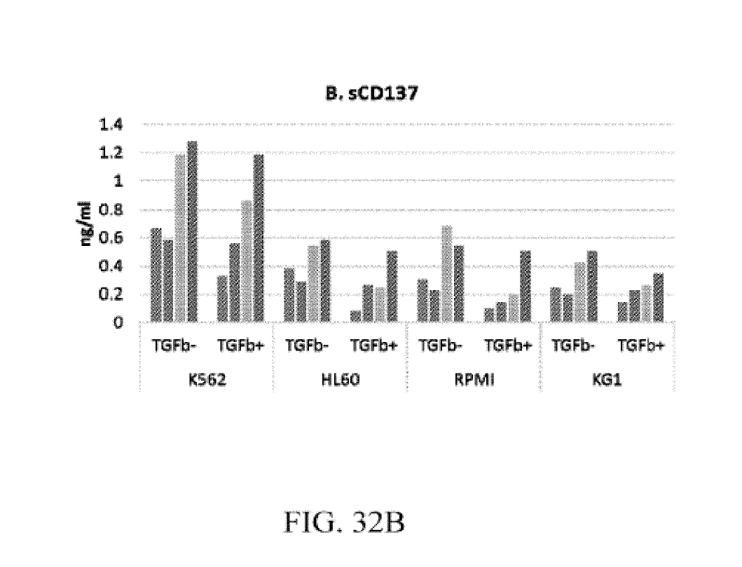

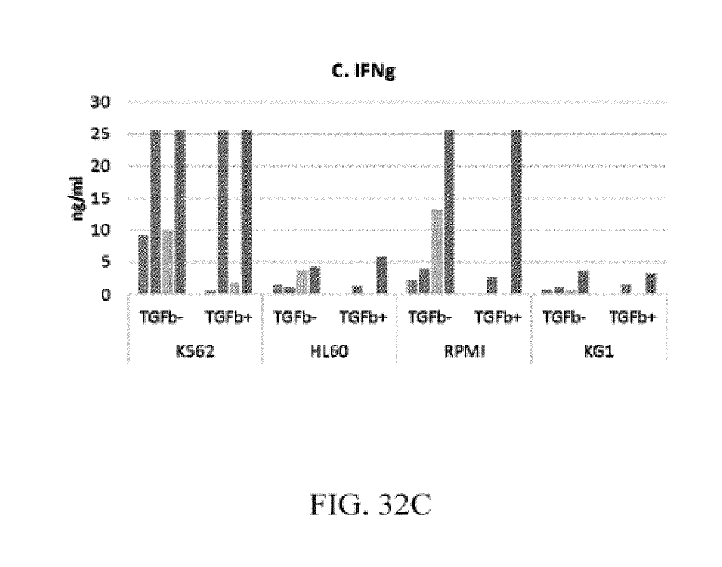

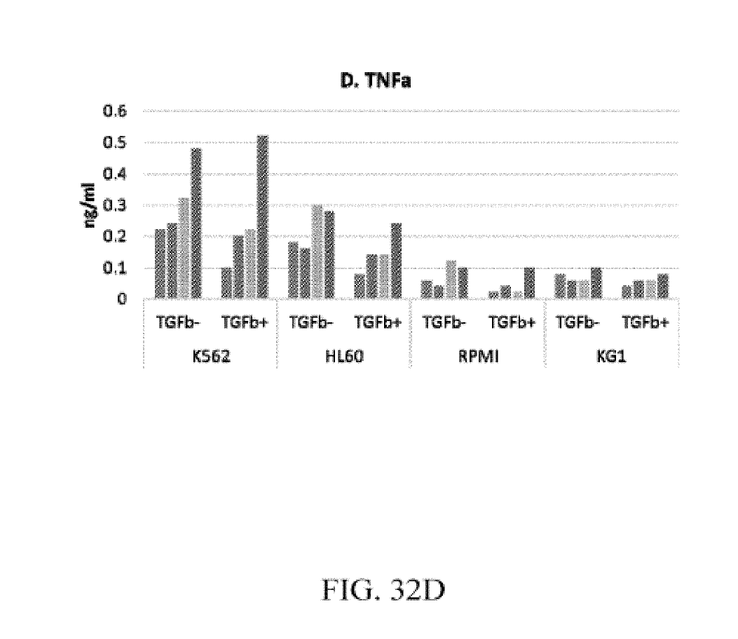

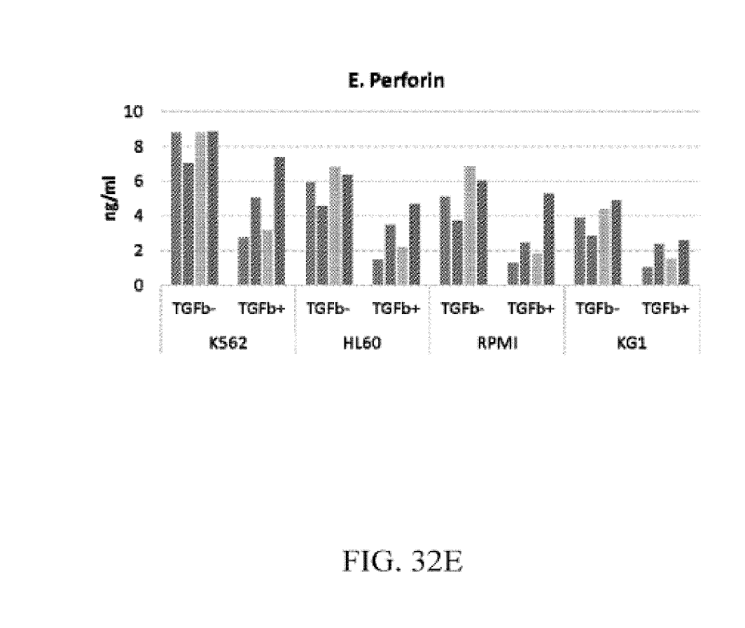

Secreted analytes from CBLB-GM NK mirrored its augmented effector function. A large increase of sCD137 and moderate increase in GM-CSF, IFNγ, TNFα and perform were observed in CBLB-GM NK when compared to control group. Secretion of these analytes was significantly reduced by TGFβ treatment in both CBLB-GM NK and GM NK control (FIG. 32A-E).

Compared to GM NK control, TGFBR2-GM NK secreted not only similar level of GM-CSF, sCD137, TNFα and perform but also greatly increased IFNγ against certain target cells. Secretion of these analytes was not inhibited by TGFβ treatment (FIG. 32A-E).

Double knock out GM NK demonstrated combined benefits from both single knock outs. Secreted analytes such as GM-CSF, sCD137, IFNγ, TNFα and perform, were not only increased but also resistant to TGFβ-triggered reduction. Synergistic effects were also observed for GM-CSF, IFNγ and TNFα. In those cases, double knock out GM NK secreted equal or greater analytes than both single knock out combined (FIG. 32A-E).

f. 7.6. Alternative 6: PNK-CD16VP

i. Background for CD16VP Construct and Experimental Setup

A CD16 construct was created for overexpression in PNK (placenta-derived NK cells) cells to generate genetic modification PNK cells with augmented ADCC function. The CD16 was created with two point mutations, one to create a high affinity Valine variant (158V/V) and second to render CD16 uncleavable by Adam 17 (S197P). The CD16 variant was termed CD16VP. Lentiviral vector was generated and CD34 cells were transduced on day 5 of expansion process. Expression of CD16 monitored during culture and function evaluated at the end of culture period. The PNK cells with or without CD16VP were tested for improvement in affinity for IgG1k antibody as well as resistance to activation induced shedding.

ii. Transduction Efficiency, PNK Expansion and Phenotype

Objective 1: To achieve high expression efficiency of CD16VP on PNK cells using lentiviral vector.

Methodology: In order to achieve high expression of CD16VP on PNK cells, CD34 cells were transduced by various conditions listed below:

Days 5 and 10 or culture

1-2 rounds of infection

Multiplicities of infection (MOI) ranging from 5 MOI to 200 MOI

Spinoculation centrifugation speeds of 600 g and 1200 g

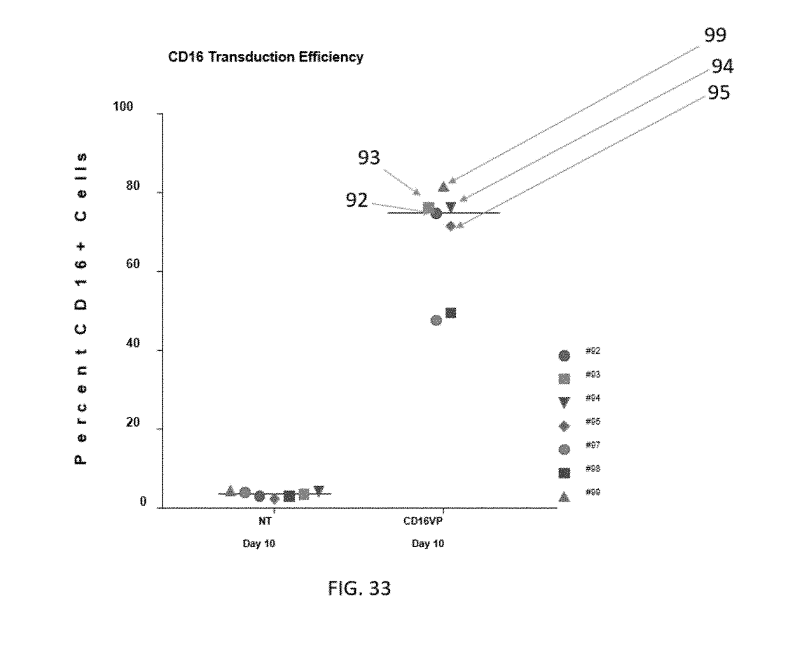

Result: Transduction on Days 5 and 10 yielded similar transduction efficiency, therefore day 5 was chosen as a standard timeframe for lentiviral transduction. The number of rounds of transduction (1 vs 2) showed minor improvement at lower MOI (50 MOI) however the efficiency was not different at higher MOI (100 MOI). The transduction efficiency increased from 50 to 100 MOI but showed no further improvement at 200 MOI. The centrifugation speed of 600 g yielded similar transduction efficiency as 1200 g. Thus the optimized protocol was determined to be spinoculation protocol at 600 g/1 hr with a single round of infection at 100MOI on day 5. The transductions were performed using non-tissue culture treated 48 well plates coated with 10-20 μg/cm2 retronectin. (FIG. 33).

The optimized transduction protocol was evaluated with CD34 donors (n=7) and a median transduction efficiency over 70% was achieved.

Objective 2: To evaluate the impact of gene modification and transduction process on expansion potential of PNK cells.

Methodology: Following the optimized transduction process, the cells were cultured as previously reported.

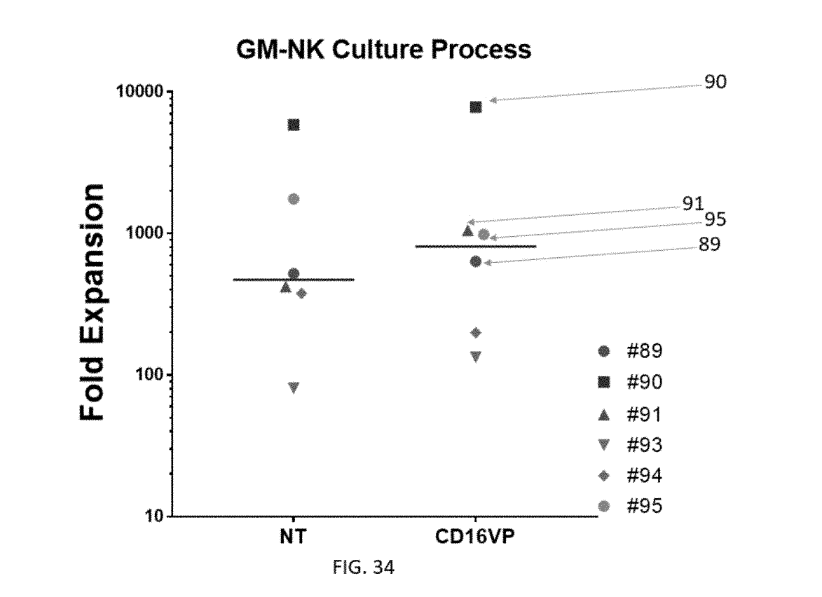

Results: The optimized transduction process did not impact the median expansion potential of the cells (n=6) even though some donors showed decrease in fold expansion by PNK-CD16VP compared to non-transduced control. (FIG. 34). The range of expansion for PNK-NT was 81-5863 and PNK-CD16VP was 134-7818 folds.

Objective 3: To evaluate the impact of lentiviral gene modification on PNK cell phenotype.

Methodology: As reported before the PNK cells were assessed for the expression of CD3, CD56, CD11a, and CD16 by flow cytometry.

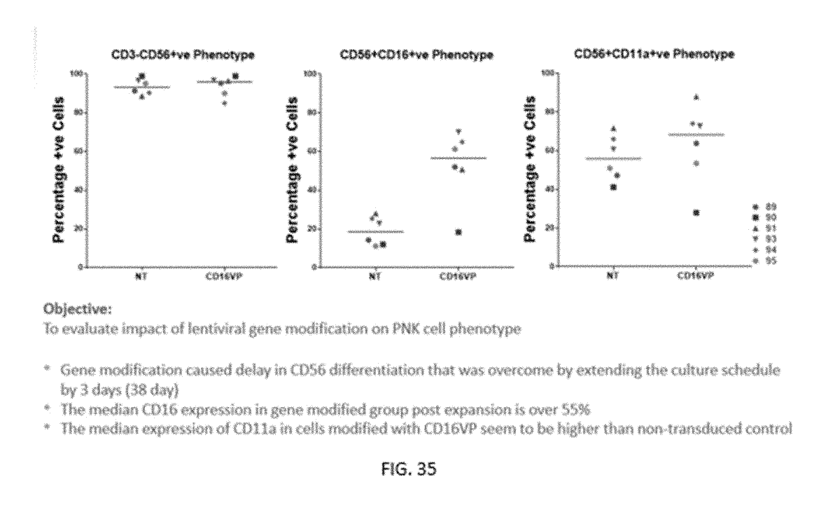

Results: Lentiviral gene modification caused a slight delay in emergence of CD56+ve cells as per the product definition criterion (of 85% CD3⁻CD56⁺ by day 35). An increase in culture duration by 3 days, from 35 to 38 days resulted in improved percent CD3⁻CD56⁺ phenotype over 85% threshold. The CD16 expression continued to be higher than non-transduced cells with a median CD16 expression in about 55% of PNK cells. A minor increase in CD11a+ve population in PNK-CD16VP was also observed compared to PNK-NT. (FIG. 35). Gene modification caused a delay in CD56 differentiation that was overcome by extending the culture schedule by 3 days (38 day). The median CD16 expression in gene modified group post expansion was shown to be over 55. The median expression of CD11a in cells modified with CD16VP seem to be higher than non-transduced control.

Delay in differentiation of CD56+PNK cell caused by gene modification was overcome by prolonging the culture duration by 3 days.

iii. PNK CD16VP Construct Validation: CD16 Induced Degranulation and Resistance to Activation Induced Cleavage

Objective 1: To evaluate functional response of CD16VP construct toward IgG1 kappa therapeutic antibodies.

Methodology: The PNK cells derived with CD16VP expression were tested for responsiveness to plate bound Unituxin antibody in a 4-hour degranulation assay. Varying concentration of Unituxin (0 μg/ml, 0.01 μg/ml, 0.1 μg/ml and 1 μg/ml) were coated onto high binding flat bottom 96 well plate at 4° C. overnight. After washing the plate with PBS, PNK-NT or PNK-CD16VP cells were seeded in presence of CD107a-PE antibody and Monensin (BD Biosciences). Following a 4-hour stimulation at 37° C. in CO2 incubator the cells were stained with CD56-APC, CD16-BV421, CD11a-FITC and CD107a-PE (all antibodies from BD Biosciences) to evaluate degranulation by PNK cells. The cells were washed, fixed and transferred to U bottom 96 well plate and read using FACSCanto I flow cytometer.

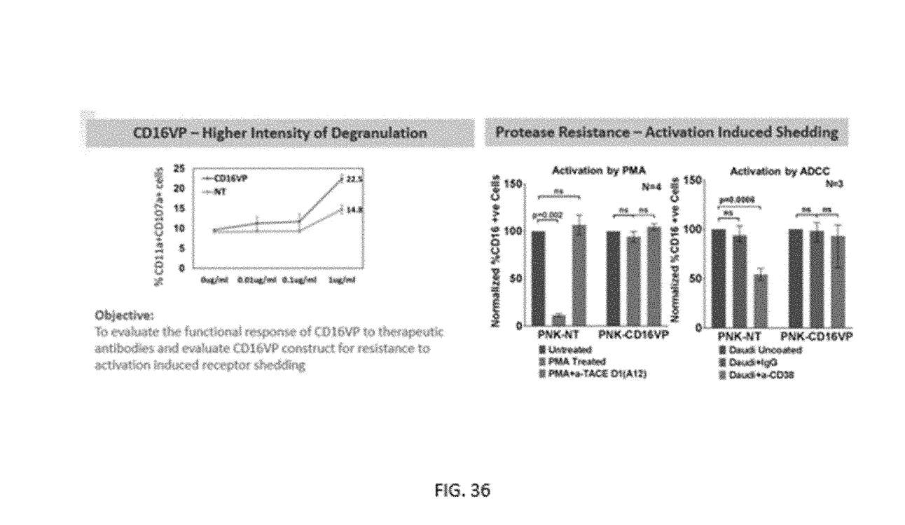

Result: Following activation with plate bound GD2 antibodies (Unituxin), both PNK-NT and PNK-CD16VP showed degranulation response demonstrated by the expression of CD107a on PNK cells at 1 ug/ml of Unituxin coating compared to uncoated wells. Increased expression of CD16 through gene modification (CD16VP) lead to increased degranulation response by PNK-CD16VP compared to PNK-NT confirming surprising results of functional intactness of the engineered CD16VP protein. (FIG. 36).

Objective 2: To evaluate the CD16VP construct for resistance to activation induced shedding.

Methodology: The PNK cells derived with CD16VP expression were tested for resistance to activation induced cleavage. The PNK cells were treated for 4 hours with immune cell activator phorbal 12-myristate 13-acetate (PMA) in the presence or absence of ADAM-17 inhibitory antibody (anti-TACE, Clone D1(A12)). The expression of CD16 was assessed using anti-CD16 antibody.

Result: Following PMA mediated activation, the non-transduced PNK (PNK-NT) cells showed a dramatic loss of CD16 expression by about 90%, which was prevented in the presence of ADAM-17 inhibitor anti-tace antibody. There was no significant loss in the expression of CD16 from PNK-CD16VP cells upon stimulation with PMA, (˜6% loss of expression). Here too the presence of ADAM-17 inhibitor prevented the observed 6% loss of CD16 expression. The data indicates that the CD16VP construct is resistant to shedding upon activation of PNK cells. The mechanism of action of PMA mediated shedding of the wildtype CD16 in PNK-NT could be attributed to the action of ADAM-17 (as demonstrated in literature) evidenced by the ability of ADAM-17 inhibitor in preventing the loss of wildtype CD16 expression.

The engineered protein CD16VP was functionally intact—able to elicit degranulation response by PNK-CD16VP and as expected was resistant to activation mediated receptor cleavage.

iv. Antibody-Dependent Cellular Cytotoxicity (ADCC)

Objective: To assess the improved ADCC potential of PNK-CD16VP

Methodology: The ADCC assay was set up as previously described, the tumor targets were pre-stained with PKH-26 dye and then stained with 20 ug/ml of therapeutic antibodies (CD20, CD38 and CD319) for 30 minutes in assay buffer 37° C. and washed to remove excess unbound antibodies. The assay was set up in U bottom 96 well plate at E:T ratios of 10:1 and 2.5:1.

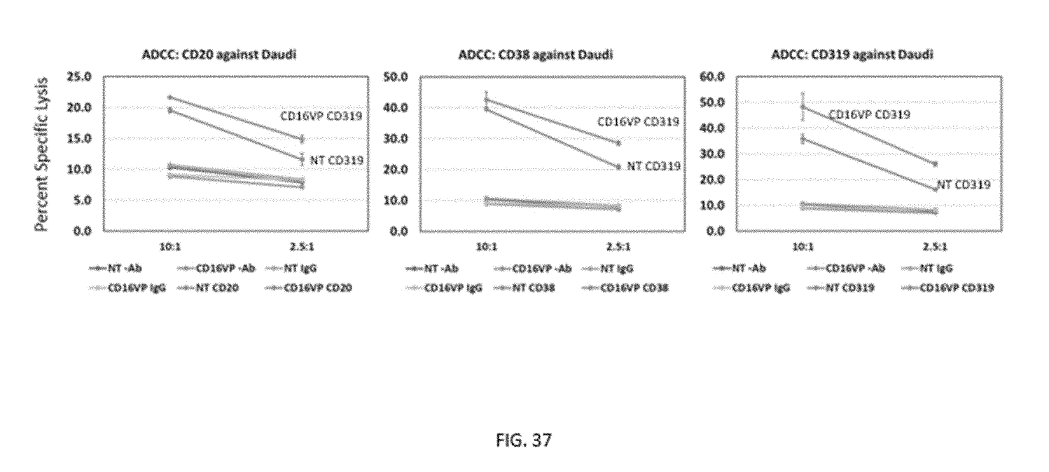

Results: The three antibodies tested showed improvement in lysis of tumor target Daudi by PNK-CD16VP compared to PNK-NT. (FIG. 37)

As shown, from the tests using pNK cells, these cells may be expanded, characterized and yield a product that may be used for treatment of diseases, such as cancer.

Optimized lentiviral transduction process was developed and a median of 70% (43-81%) transduction efficiency was achieved and maintained a median expression over 50% at the end of expansion. The experiments also show that the transduction process did not negatively impact the median expansion potential of PNK-CD16VP cells compared to PNK-NT (range from 81-7818).

The differentiation of CD56+ve PNK cells was slightly delayed which was overcome by extending the culture duration by 3 days

However, the additional expression of CD16 on PNK-CD16VP led to a higher degranulation by PNK-CD16VP in response to plate bound therapeutic IgG1 Kappa antibody Unituxin—indicating functional intactness of the engineered protein.

The CD16VP was confirmed to be resistant to activation induced shedding/cleavage

Surprisingly, the PNK-CD16VP cells showed higher cytotoxicity against Daudi tumor line against CD20, CD38 and CD319 antibodies.

The present invention is not to be limited in scope by the specific alternatives described herein. Indeed, various modifications of the invention in addition to those described will become apparent to those skilled in the art from the foregoing description and accompanying figures. Such modifications are intended to fall within the scope of the appended claims.

All references cited herein are incorporated herein by reference in their entirety and for all purposes to the same extent as if each individual publication, patent or patent application was specifically and individually indicated to be incorporated by reference in its entirety for all purposes. The citation of any publication is for its disclosure prior to the filing date and should not be construed as an admission that the present invention is not entitled to antedate such publication by virtue of prior invention.

What is claimed is:

1. A population of natural killer cells, wherein the natural killer (NK) cells are genetically modified to lack expression of an NK inhibitory molecule or manifest a reduced expression of an NK inhibitory molecule.

2. The population of claim 1, wherein the NK inhibitory molecule is one or more NK inhibitory molecules selected from the group consisting of CBLB, NKG2A and TGFBR2.

3. The population of claim 1, wherein the genetically modified NK cells have a higher cytotoxicity against tumor cells than NK cells in which expression of the NK inhibitory molecule has not been knocked out or reduced.

4. The population of claim 3, wherein the tumor cells are selected from the group consisting of multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, retinoblastoma cells and solid tumor cells, wherein the solid tumor cells are selected from the group consisting of liver tumor cells, lung tumor cells, pancreatic tumor cells, renal tumor cells, and glioblastoma multiforme (GBM) cells.

5. The population of claim 1, wherein expression of the NK inhibitory molecule has been knocked out, wherein the NK inhibitory molecule is CBLB, NKG2A, or TGFBR2.

6. The population of claim 5,
wherein the knockout of CBLB expression generates a population of NK cells having a higher IFNγ secretion when stimulated with ICAM-1 and MICA than NK cells in which CBLB has not been knocked out or wherein the knockout of CBLB expression generates a population of NK cells having a higher degranulation when stimulated with ICAM-1 and MICA than NK cells in which CBLB has not been knocked out or wherein the knockout of CBLB expression generates a population of NK cells having a change in the secretion of one or more of GM-CSF, soluble CD137 (sCD137), IFNγ, MIP1α, MIP1β, TNFα and perform when co-cultured with multiple myeloma cells, compared to NK cells in which CBLB has not been knocked out;
wherein the knockout of NKG2A expression generates a population of NK cells having a higher degranulation when stimulated with ICAM-1 and MICA in the presence of an NKG2A agonist antibody than NK cells in which NKG2A has not been knocked out or wherein the knockout of NKG2A expression generates a population of NK cells having a change in the secretion of one or more of GM-CSF, soluble CD137 (sCD137), IFNγ, MIP1α, MIP1β, TNFα and/or perform, compared to NK cells in which NKG2A has not been knocked out; and
wherein the knockout of TGFBR2 expression generates a population of NK cells having a resistance to TGFβ mediated inhibition of NK cell cytotoxicity against tumor cells compared to NK cells in which TGFBR2 has not been knocked out.

7. The population of claim 1, wherein the NK cells are placenta derived (PNK cells).

8. A population of natural killer cells, wherein the natural killer (NK) cells are genetically modified to comprise a modified CD16.

9. The population of claim 8, wherein the modified CD16 has a higher affinity for IgG than wildtype CD16.

10. The population of claim 9, wherein the modified CD16 has a valine at position 158 of CD16a and a proline at position 197 of CD16a.

11. The population of claim 8, wherein the modified CD16 is introduced into the NK cells via viral infection.

12. The population of claim 8, wherein the NK cells are placenta derived (PNK cells).

13. A method of suppressing the proliferation of tumor cells comprising contacting the tumor cells with natural killer cells from the population of claim 1.

14. The method of claim 13, wherein said contacting takes place in vitro or in vivo.

15. The method of claim 13, wherein said contacting takes place in a human individual, preferably an individual selected to receive an anticancer therapy.

16. The method of claim 15, wherein said method comprises administering said natural killer cells to said individual, wherein said individual has AML that has failed at least one non-innate lymphoid cell (ILC) therapeutic against AML or wherein said individual has AML that has failed at least one non-innate lymphoid cell (ILC) therapeutic against AML or wherein said individual has relapsed/refractory AML or wherein said individual is 65 years old or greater, and is in first remission.

17. The method of claim 13, wherein said tumor cells are multiple myeloma cells, acute myeloid leukemia (AML) cells, breast cancer cells, head and neck cancer cells, sarcoma cells, ductal carcinoma cells, leukemia cells, acute T cell leukemia cells, chronic myeloid lymphoma cells, chronic myelogenous leukemia (CML) cells, multiple myeloma (MM), lung carcinoma cells, colon adenocarcinoma cells, histiocytic lymphoma cells, colorectal carcinoma cells, colorectal adenocarcinoma cells, retinoblastoma cells or solid tumor cells.

18. The method of claim 13, wherein said natural killer cells are administered with an anti-CD33 antibody, anti-CD20 antibody, an anti-CD138 antibody, or anti-CD38 antibody.

19. The population of claim 1, wherein the natural killer cells are CD56⁺CD3⁻CD117⁺CD11a⁺, express perform and/or EOMES, and do not express one or more of RORγt, aryl hydrocarbon receptor, and IL1R1.

20. The population of 19, wherein said natural killer cells additionally express T-bet, GZMB, NKp46, NKp30, and/or NKG2D.

21. A method of suppressing the proliferation of tumor cells comprising contacting the tumor cells with natural killer cells from the population of claim 8.

22. A population of natural killer cells derived from placenta or parts thereof, thereby comprising placenta derived NK cells (pNK cells), wherein the pNK cells are genetically modified such that they lack expression of an NK inhibitory molecule or manifest reduced expression of an NK inhibitory molecule, wherein expression of the NK inhibitory molecule has been knocked out, wherein the NK inhibitory molecule is CBLB, NKG2A or TGFBR2.

23. A population of placental derived natural killer cells (pNK), wherein the pNK cells are genetically modified to comprise a modified CD16.

24. The population of claim 23, wherein the modified CD16 has a higher affinity for IgG than wildtype CD16.

25. The population of claim 24, wherein the modified CD16 has a valine at position 158 of CD16a and a proline at position 197 of CD16a.
